(19) United States
(12) Patent Application Publication
(10) Pub. No.: US 2021/0379149 A1
(43) Pub. Date: Dec. 9, 2021

(54) Increasing or Maintaining T-Cell Subpopulations in Adoptive T-Cell Therapy

(71) Applicants: Innovative Cellular Therapeutics Co., Ltd., Shanghai (CN); Innovative Cellular Therapeutics, Inc., Rockville, MD (US)

(72) Inventors: Chengfei Pu, Shanghai (CN); Zhiyuan Cao, Shanghai (CN); He Sun, Shanghai (CN); Zhao Wu, Shanghai (CN); Lei Xiao, Rockville, MD (US)

(73) Assignees: Innovative Cellular Therapeutics Holdings, Ltd., Shanghai (CN); Innovative Cellular Therapeutics, Inc., Rockville, MD (US)

(21) Appl. No.: 17/287,862

(22) PCT Filed: Oct. 25, 2019
(86) PCT No.: PCT/US19/58121

§ 371 Date: Apr. 22, 2021

Related U.S. Application Data
(60) Provisional application No. 62/750,455, filed on Oct. 25, 2018.
(60) Provisional application No. 62/802,951, filed on Feb. 8, 2019.
(60) Provisional application No. 62/825,420, filed on Mar. 28, 2019.

(51) Int. Cl. A61K 38/17; A61P 35/00; A61K 35/17; A61K 39/395

(52) U.S. Cl. CPC A61K 38/1774; A61P 35/00; A61K 38/177; A61K 39/3955; A61K 39/39558; A61K 35/17

(57) ABSTRACT

Embodiments relate to a method for increasing or maintaining a population of memory T-cells. The method includes introducing T-cells with a nucleic acid encoding a chimeric antigen receptor (CAR), wherein the CAR includes an extracellular domain, a transmembrane domain, and an intracellular domain, and the extracellular domain binds an antigen; introducing into the T-cells a nucleic acid encoding a modified PD-1, wherein the modified PD-1 lacks a functional PD-1 intracellular domain; and administering an effective amount of a composition comprising the T-cells to a subject having cancer.

### CROSS REFERENCE TO RELATED APPLICATIONS

This application claims the benefit of U.S. Provisional Application No. 62/825,420 filed Mar. 28, 2019; U.S. Provisional Application No. 62/802,951 filed Feb. 8, 2019 (0043USP2); and U.S. Provisional Application No. 62/750,455 filed Oct. 25, 2018 (0043USP1), which are incorporated herein by reference in their entirety.

### SEQUENCE LISTING INFORMATION

A computer readable textfile, entitled “Sequence listing.txt,” created on or about Oct. 11, 2019 with a file size of about 119 KB, contains the sequence listing for this application and is hereby incorporated by reference in its entirety.

### TECHNICAL FIELD

The present disclosure relates to modified cells and uses, in particular to compositions and methods for treating cancer using Chimeric Antigen Receptor (CAR) cells.

### BACKGROUND

In immune therapy, effective T-cell response plays an important role against infection and cancer. It has been reported that checkpoint molecules such as PD1/PD-L1 signaling may suppress T-cell response in immune therapy; therefore, blocking PD1/PD-L1 may enhance T-cell response. However, it has been shown that the deletion or genetic absence of PD-1 or other such molecules in T-cells in vivo may negatively impact long-lived memory either by preventing reprogramming or persistence which effects long-term efficacy of T-cell based therapy.

### SUMMARY

Embodiments relate to a method for increasing a number and/or ratio of a subpopulation of immune cells infused into a subject having cancer and/or increasing or extending persistence of a subpopulation of immune cells infused into the subject, the method comprising: administering an effective amount of a composition comprising T-cells to the subject, the T-cells including an antigen binding molecule (e.g., chimeric antigen receptor (CAR)) and a modified PD-1, the modified PD-1 lacking a functional PD-1 intracellular domain; and monitoring the T-cell response in the subject, the subpopulation of immune cells comprising naive T-cells, stem cell memory T-cells, and/or central memory T-cells, wherein the number and/or ratio of the immune cell subpopulation in the subject is increased as compared to the number and/or ratio in a subject administered with corresponding T-cells that do not include the modified PD-1 or wherein the persistence of the subpopulation of immune cells in the subject is increased or extended as compared to the persistence of the subpopulation of immune cells in a subject administered with corresponding T-cells that do not include the modified PD-1.

This Summary is not intended to identify key features or essential features of the claimed subject matter, nor is it intended to be used to limit the scope of the claimed subject matter.

### BRIEF DESCRIPTION OF THE DRAWINGS

The Detailed Description is described with reference to the accompanying figures. The use of the same reference numbers in different figures indicates similar or identical items.

FIG. 1 shows flow cytometry results of modified PD-1 and CAR expression. DnPD-1 (PD-1 WT extracellular domain+PD-1 WT transmembrane domain+SEQ ID NO: 36).

FIGS. 2, 3, 4, 5, and 6 show the results of killing assay.

FIG. 7 shows the results of tumor challenging assay.

FIG. 8 shows CAR T-cell type analysis when they were cultured alone. hCD19 CAR+dnPD-1 (Antigen binding domain of CD19 CAR: SEQ ID NO: 5 and DnPD1: SEQ ID NO: 36). DnPD-1 (PD-1 WT extracellular domain+PD-1 WT transmembrane domain+SEQ ID NO: 36).

FIG. 9 shows a histogram of exogenous PD-1 mRNA levels in various CAR T-cells. DnPD-1 (PD-1 WT extracellular domain+PD-1 WT transmembrane domain+SEQ ID NO: 36). (Sequence Infor: CD19 CAR including SEQ ID NO: 5, 43, and 53; DnPD1 including SEQ ID NO: 36 or 37)

FIG. 10 shows a histogram of endogenous PD-1 mRNA levels in various CAR T-cells. DnPD-1 (PD-1 WT extracellular domain+PD-1 WT transmembrane domain+SEQ ID NO: 36). (Sequence Infor: CD19 CAR including SEQ ID NO: 5, 43, and 53; DnPD1 including SEQ ID NO: 36 or 37). DnPD-1 (PD-1 WT extracellular domain+PD-1 WT transmembrane domain+SEQ ID NO: 36).

FIGS. 11A and 11B show results of tumor challenging assay showing 19CAR+dnPD1 T-cells are more persistent than 19CAR T-cells in response to the tumor challenging.

FIG. 12 shows results of CAR T-cell phenotype analysis on two patients (P1 and P2).

FIG. 13 shows the PET-Scan results of the two patients (P1 and P2) who received treatment of hCD19 CAR+dnPD-1 T-cells. (Sequence Infor: CD19 CAR including SEQ ID NO: 5, 43, and 53; DnPD-1 including SEQ ID NO: 36 or 37).

FIGS. 14 and 15 show the results of cell analysis after CAR T-cell infusion of patient P1.

FIGS. 16 and 17 show the results of cell analysis after CAR T-cell infusion of patient P2.

FIG. 18 shows CAR and PD-1 expression and copy numbers of anti-CD19 CAR and Anti-CD19 DnPD-1 CAR T-cells.

FIG. 19 shows information of a third patient (P3) and the patient's PET CT scanning results at 31 days after infusion. P3 was infused with DnPD-1 (PD-1 WT extracellular domain+PD-1 WT transmembrane domain+SEQ ID NO: 36).

FIG. 20 shows the clinical study scheme for the clinical trials in FIGS. 14-17 and 19

FIG. 21 shows expression of PD-1 of CAR T and dnPD-1 CAR T-cells.

FIGS. 22 and 23 show the scheme of an in vivo embodiment.

FIG. 24 shows T-cell expansion and IFN-γ release from cultured cells of patient P4 having breast cancer before the infusion.

FIGS. 25 and 26 show the results of cell analysis after CAR T-cell infusion of patient P4.

FIG. 27 shows a schematic diagram showing an exemplary portion of a cell membrane comprising two CAR molecules.

FIG. 28 shows a schematic diagram showing an exemplary portion of a cell membrane comprising a bispecific CAR molecule and a T-cell receptor (TCR).

FIG. 29 shows a schematic diagram showing an exemplary portion of a cell membrane comprising a bispecific CAR molecule.

FIG. 30 shows a schematic diagram showing an exemplary portion of a targeting vector.

FIG. 31 shows a schematic diagram showing an exemplary portion of two targeting vectors.

FIG. 32 shows a schematic diagram showing an exemplary portion of two targeting vectors.

FIG. 33 shows ex-vivo anti-tumor effect of CAR19-dnPD-1 versus CAR19 (without dnPD-1). Left: Percentage of remaining GFP-positive tumor cell (Nalm6 and Nalm6-PD1) after co-culture with CAR19 and CAR19-dnPD-1- in continuous 6 rounds experiments

FIG. 34 shows flow cytometry results confirming that dnPD-1 CAR T-cells show enhanced capability of tumor killing after multiple-rounds of tumor challenge and exhibit more “memory-like” phenotypes.

FIG. 35 shows CAR copy number changes in patients P1 and P2 after dnPD-1 CD19 CAR T infusion and endogenous PD-1 as well as exogenous PD-1 expression.

### DETAILED DESCRIPTION

The deletion or genetic absence of PD-1 or other such molecules in T-cells in vivo may also negatively impact the long-lived memory compartment, either by preventing reprogramming or persistence while in some contexts despite preventing an exhausted phenotype and permitting primary expansion and activation, and other effector functions. The long-lived memory T-cells are important to in vivo persistence of T-cell response in immune therapy. Additionally, the differentiation state of the administered T-cells can significantly affect in vivo persistence and anti-tumor activity. T helper (CD4+ T-cells) and cytotoxic T-cells (CD8+), specifically, naive (Tn), stem cell memory (Tscm) and central memory (Tcm) T-cells, characterized by the expression of the CCR7 and CD62L markers, mediate superior anti-tumor activity in both mouse models (Sommermeyer et al. 2016) and in non-human primate models (Berger et al 2008). Under conventional techniques, it seems challenging to block PD-1/PD-L1 signaling to enhance T-cell response in immune therapy without a negative impact on the persistence of the T-cell response.

Embodiments relate to the surprising discovery that blocking PD-1-PD-L1 signaling using dominant negative PD-1 techniques may increase or extend the persistence of T-cell response by increasing a number and/or ratio of a subpopulation of immune cells infused into a subject and/or increasing or extending persistence of a subpopulation of immune cells infused into the subject.

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, preferred methods and materials are described. For the purposes of the present disclosure, the following terms are defined below.

The articles “a” and “an” are used herein to refer to one or to more than one (i.e., to at least one) of the grammatical object of the article. By way of example, “an element” means one element or more than one element.

By “about” is meant a quantity, level, value, number, frequency, percentage, dimension, size, amount, weight or length that varies by as much as 20, 15, 10, 9, 8, 7, 6, 5, 4, 3, 2 or 1% to a reference quantity, level, value, number, frequency, percentage, dimension, size, amount, weight or length. Unless otherwise indicated, all numbers expressing quantities used throughout the specification and claims are to be understood as being modified in all instances by the term “about.”

The term “activation,” as used herein, refers to the state of a cell that has been sufficiently stimulated to induce detectable cellular proliferation. Activation can also be associated with induced cytokine production and detectable effector functions. The term “activated T-cells” refers to, among other things, T-cells that are undergoing cell division.

The term “antibody” is used in the broadest sense and refers to monoclonal antibodies (including full length monoclonal antibodies), polyclonal antibodies, multi-specific antibodies (e.g., bispecific antibodies), and antibody fragments so long as they exhibit the desired biological activity or function. The antibodies in the present disclosure may exist in a variety of forms including, for example, polyclonal antibodies, monoclonal antibodies, Fv, Fab and F(ab)2, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “antibody fragments” refers to a portion of a full length antibody, for example, the antigen binding or variable region of the antibody. Other examples of antibody fragments include Fab, Fab′, F(ab′)2, and Fv fragments; diabodies; linear antibodies; single-chain antibody molecules; and multi-specific antibodies formed from antibody fragments.

The term “Fv” refers to the minimum antibody fragment which contains a complete antigen-recognition and -binding site. This fragment consists of a dimer of one heavy- and one light-chain variable region domain in tight, non-covalent association. From the folding of these two domains emanates six hypervariable loops (3 loops each from the H and L chain) that contribute the amino acid residues for antigen binding and confer antigen binding specificity to the antibody. However, even a single variable domain (or half of a Fv including only three complementarity determining regions (CDRs) specific for an antigen) has the ability to recognize and bind antigen, although at a lower affinity than the entire binding site (the dimer).

An “antibody heavy chain,” as used herein, refers to the larger of the two types of polypeptide chains present in all antibody molecules in their naturally occurring conformations. An “antibody light chain,” as used herein, refers to the smaller of the two types of polypeptide chains present in all antibody molecules in their naturally occurring conformations. K and A light chains refer to the two major antibody light chain isotypes.

The term “synthetic antibody” refers to an antibody which is generated using recombinant DNA technology, such as, for example, an antibody expressed by a bacteriophage. The term also includes an antibody which has been generated by the synthesis of a DNA molecule encoding the antibody and the expression of the DNA molecule to obtain the antibody, or to obtain an amino acid encoding the antibody. The synthetic DNA is obtained using technology that is available and well known in the art.

The term “antigen” refers to a molecule that provokes an immune response, which may involve either antibody production, or the activation of specific immunologically-competent cells, or both. Antigens include any macromolecule, including all proteins or peptides, or molecules derived from recombinant or genomic DNA. For example, DNA including a nucleotide sequence or a partial nucleotide sequence encoding a protein or peptide that elicits an immune response, and therefore, encodes an “antigen” as the term is used herein. An antigen need not be encoded solely by a full-length nucleotide sequence of a gene. An antigen can be generated, synthesized or derived from a biological sample including a tissue sample, a tumor sample, a cell, or a biological fluid.

The term “anti-tumor effect” as used herein, refers to a biological effect associated with a decrease in tumor volume, a decrease in the number of tumor cells, a decrease in the number of metastases, decrease in tumor cell proliferation, decrease in tumor cell survival, an increase in life expectancy of a subject having tumor cells, or amelioration of various physiological symptoms associated with the cancerous condition. An “anti-tumor effect” can also be manifested by the ability of the peptides, polynucleotides, cells, and antibodies in the prevention of the occurrence of tumor in the first place.

The term “auto-antigen” refers to an endogenous antigen mistakenly recognized by the immune system as being foreign. Auto-antigens include cellular proteins, phosphoproteins, cellular surface proteins, cellular lipids, nucleic acids, glycoproteins, including cell surface receptors.

The term “autologous” is used to describe a material derived from a subject which is subsequently re-introduced into the same subject.

The term “allogeneic” is used to describe a graft derived from a different subject of the same species. As an example, a donor subject may be a related or unrelated to the recipient subject, but the donor subject has immune system markers which are similar to the recipient subject.

The term “xenogeneic” is used to describe a graft derived from a subject of a different species. As an example, the donor subject is from a different species than a recipient subject, and the donor subject and the recipient subject can be genetically and immunologically incompatible.

The term “cancer” as used to refer to a disease characterized by the rapid and uncontrolled growth of aberrant cells. Cancer cells can spread locally or through the bloodstream and lymphatic system to other parts of the body. Examples of various cancers include breast cancer, prostate cancer, ovarian cancer, cervical cancer, skin cancer, pancreatic cancer, colorectal cancer, renal cancer, liver cancer, brain cancer, lymphoma, leukemia, lung cancer, and the like.

Throughout this specification, unless the context requires otherwise, the words “comprise,” “includes” and “including” will be understood to imply the inclusion of a stated step or element (ingredient or component) or group of steps or elements (ingredients or components) but not the exclusion of any other step or element or group of steps or elements.

The phrase “consisting of” is meant to include, and is limited to, whatever follows the phrase “consisting of.” Thus, the phrase “consisting of” indicates that the listed elements are required or mandatory and that no other elements may be present.

The phrase “consisting essentially of” is meant to include any element listed after the phrase and can include other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The terms “complementary” and “complementarity” refer to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules, or there may be “complete” or “total” complementarity between the nucleic acids. The degree of complementarity between nucleic acid strands has significant effects on the efficiency and strength of hybridization between nucleic acid strands.

The term “corresponds to” or “corresponding to” refers to (a) a polynucleotide having a nucleotide sequence that is substantially identical or complementary to all or a portion of a reference polynucleotide sequence or encoding an amino acid sequence identical to an amino acid sequence in a peptide or protein; or (b) a peptide or polypeptide having an amino acid sequence that is substantially identical to a sequence of amino acids in a reference peptide or protein.

The term “co-stimulatory ligand,” refers to a molecule on an antigen presenting cell (e.g., an APC, dendritic cell, B cell, and the like) that specifically binds a cognate co-stimulatory molecule on a T-cell, thereby providing a signal which, in addition to the primary signal provided by, for instance, binding of a TCR/CD3 complex with an MHC molecule loaded with peptide, mediates a T-cell response, including at least one of proliferation, activation, differentiation, and other cellular responses. A co-stimulatory ligand can include B7-1 (CD80), B7-2 (CD86), PD-L1, PD-L2, 4-1BBL, OX40L, inducible co-stimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, a ligand for CD7, an agonist or antibody that binds the Toll ligand receptor and a ligand that specifically binds with B7-H3. A co-stimulatory ligand also includes, inter alia, an agonist or an antibody that specifically binds with a co-stimulatory molecule present on a T-cell, such as CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds CD83.

The term “co-stimulatory molecule” refers to the cognate binding partner on a T-cell that specifically binds with a co-stimulatory ligand, thereby mediating a co-stimulatory response by the T-cell, such as proliferation. Co-stimulatory molecules include an MHC class I molecule, BTLA, and a Toll-like receptor.

The term “co-stimulatory signal” refers to a signal, which in combination with a primary signal, such as TCR/CD3 ligation, leads to T-cell proliferation and/or upregulation or downregulation of key molecules. The terms “disease” and “condition” may be used interchangeably or may be different in that the particular malady or condition may not have a known causative agent (so that etiology has not yet been worked out), and it is therefore not yet recognized as a disease but only as an undesirable condition or syndrome, wherein a more or less specific set of symptoms have been identified by clinicians. The term “disease” is a state of health of a subject wherein the subject cannot maintain homeostasis, and wherein if the disease is not ameliorated then the subject's health continues to deteriorate. In contrast, a “disorder” in a subject is a state of health in which the animal is able to maintain homeostasis, but in which the animal's state of health is less favorable than it would be in the absence of the disorder. Left untreated, a disorder does not necessarily cause a further decrease in the animal's state of health.

The term “effective” refers to adequate to accomplish a desired, expected, or intended result. For example, an “effective amount” in the context of treatment may be an amount of a compound sufficient to produce a therapeutic or prophylactic benefit.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence (except that a “T” is replaced by a “U”) and is usually provided in sequence listings, and the non-coding strand, used as the template for transcription of a gene or cDNA, can be referred to as encoding the protein or other product of that gene or cDNA.

The term “exogenous” refers to a molecule that does not naturally occur in a wild-type cell or organism but is typically introduced into the cell by molecular biological techniques. Examples of exogenous polynucleotides include vectors, plasmids, and/or man-made nucleic acid constructs encoding the desired protein. With regard to polynucleotides and proteins, the term “endogenous” or “native” refers to naturally-occurring polynucleotide or amino acid sequences that may be found in a given wild-type cell or organism. Also, a particular polynucleotide sequence that is isolated from a first organism and transferred to a second organism by molecular biological techniques is typically considered an “exogenous” polynucleotide or amino acid sequence with respect to the second organism. In specific embodiments, polynucleotide sequences can be “introduced” by molecular biological techniques into a microorganism that already contains such a polynucleotide sequence, for instance, to create one or more additional copies of an otherwise naturally-occurring polynucleotide sequence, and thereby facilitate overexpression of the encoded polypeptide.

The term “expression” refers to the transcription and/or translation of a particular nucleotide sequence driven by its promoter.

The term “expression vector” refers to a vector including a recombinant polynucleotide including expression control (regulatory) sequences operably linked to a nucleotide sequence to be expressed. An expression vector includes sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

The term “homologous” refers to sequence similarity or sequence identity between two polypeptides or between two polynucleotides when a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared×100. For example, if 6 of 10 of the positions in two sequences are matched or homologous then the two sequences are 60% homologous. By way of example, the DNA sequences ATTGCC and TATGGC share 50% homology. A comparison is made when two sequences are aligned to give maximum homology.

The term “immunoglobulin” or “Ig,” refers to a class of proteins, which function as antibodies. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function but may serve as an antigen receptor. IgE is the immunoglobulin that mediates immediate hypersensitivity by causing the release of mediators from mast cells and basophils upon exposure to the allergen.

The term “isolated” refers to a material that is substantially or essentially free from components that normally accompany it in its native state. The material can be a cell or a macromolecule such as a protein or nucleic acid. For example, an “isolated polynucleotide,” as used herein, refers to a polynucleotide, which has been purified from the sequences which flank it in a naturally-occurring state, e.g., a DNA fragment which has been removed from the sequences that are normally adjacent to the fragment. Alternatively, an “isolated peptide” or an “isolated polypeptide” and the like, as used herein, refer to in vitro isolation and/or purification of a peptide or polypeptide molecule from its natural cellular environment, and from association with other components of the cell.

The term “substantially purified” refers to a material that is substantially frr from components that normally associated with it in its native state. For example, a substantially purified cell refers to a cell that has been separated from other cell types with which it is normally associated in its naturally occurring or native state. In some instances, a population of substantially purified cells refers to a homogenous population of cells. In other instances, this term refers simply to a cell that has been separated from the cells with which they are naturally associated in their natural state. In embodiments, the cells are cultured in vitro. In embodiments, the cells are not cultured in vitro.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. Moreover, the use of lentiviruses enables integration of the genetic information into the host chromosome resulting in stably transduced genetic information. HIV, SIV, and FIV are all examples of lentiviruses. Vectors derived from lentiviruses offer the means to achieve significant levels of gene transfer in vivo.

The term “modulating,” refers to mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation.

The term “under transcriptional control” refers to a promoter being operably linked to and in the correct location and orientation in relation to a polynucleotide to control the initiation of transcription by RNA polymerase and expression of the polynucleotide.

The term “overexpressed” tumor antigen or “overexpression” of the tumor antigen is intended to indicate an abnormal level of expression of the tumor antigen in a cell from a disease area such as a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art.

Cancers that can be treated include tumors that are not vascularized, or not yet substantially vascularized, as well as vascularized tumors. The cancers may include non-solid tumors (such as hematological tumors, for example, leukemias and lymphomas) or may include solid tumors. Types of cancers to be treated with the CARs of the disclosure include, but are not limited to, carcinoma, blastoma, and sarcoma, and certain leukemia or lymphoid malignancies, benign and malignant tumors, and malignancies, e.g., sarcomas, carcinomas, and melanomas. Adult tumors/cancers and pediatric tumors/cancers are also included.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme), astrocytoma, CNS lymphoma, germinoma, medulloblastoma, Schwannoma craniopharyngioma, ependymoma, pinealoma, hemangioblastoma, acoustic neuroma, oligodendroglioma, meningioma, neuroblastoma, retinoblastoma, and brain metastases).

A solid tumor antigen is an antigen expressed on a solid tumor. In embodiments, solid tumor antigens are also expressed at low levels on healthy tissue. Examples of solid tumor antigens and their related disease tumors are provided in Table 1.

TABLE 1

| Solid Tumor | |
|---|---|
| antigen | Disease tumor |
| PRLR | Breast Cancer |
| CLCA1 | colorectal Cancer |
| MUC12 | colorectal Cancer |
| GUCY2C | colorectal Cancer |
| GPR35 | colorectal Cancer |
| CR1L | Gastric Cancer |
| MUC 17 | Gastric Cancer |
| TMPRSS11B | esophageal Cancer |
| MUC21 | esophageal Cancer |
| TMPRSS11E | esophageal Cancer |
| CD207 | bladder Cancer |
| SLC30A8 | pancreatic Cancer |
| CFC1 | pancreatic Cancer |
| SLC12A3 | Cervical Cancer |
| SSTR1 | Cervical tumor |
| GPR27 | Ovary tumor |
| FZD10 | Ovary tumor |
| TSHR | Thyroid Tumor |
| SIGLEC15 | Urothelial cancer |
| SLC6A3 | Renal cancer |
| KISS1R | Renal cancer |
| QRFPR | Renal cancer: |
| GPR119 | Pancreatic cancer |
| CLDN6 | Endometrial cancer/Urothelial cancer |
| UPK2 | Urothelial cancer (including bladder cancer) |
| ADAM12 | Breast cancer, pancreatic cancer and the like |
| SLC45A3 | Prostate cancer |
| ACPP | Prostate cancer |
| MUC21 | Esophageal cancer |
| MUC16 | Ovarian cancer |
| MS4A12 | Colorectal cancer |
| ALPP | Endometrial cancer |
| CEA | Colorectal carcinoma |
| EphA2 | Glioma |
| FAP | Mesothelioma |
| GPC3 | Lung squamous cell carcinoma |
| IL13-Rα2 | Glioma |
| Mesothelin | Metastatic cancer |
| PSMA | Prostate cancer |
| ROR1 | Breast lung carcinoma |
| VEGFR-II | Metastatic cancer |
| GD2 | Neuroblastoma |
| FR-α | Ovarian carcinoma |
| ErbB2 | Carcinomas |
| EpCAM | Carcinomas |
| EGFRvIII | Glioma-Glioblastoma |
| EGFR | Glioma-NSCL cancer |
| tMUC 1 | Cholangiocarcinoma, Pancreatic cancer, Breast Cancer |
| PSCA | pancreas, stomach, or prostate cancer |

The term “parenteral administration” of a composition includes, e.g., subcutaneous (s.c.), intravenous (i.v.), intramuscular (i.m.), intrasternal injection, or infusion techniques.

The terms “patient,” “subject,” and “individual,” and the like are used interchangeably herein, and refer to any human or animal amenable to the methods described herein. In certain non-limiting embodiments, the patient, subject, or individual is a human or animal. In embodiments, the term “subject” is intended to include living organisms in which an immune response can be elicited (e.g., mammals). Examples of subjects include humans, and animals such as dogs, cats, mice, rats, and transgenic species thereof.

A subject in need of treatment or in need thereof includes a subject having a disease, condition, or disorder that needs to be treated. A subject in need thereof also includes a subject that needs treatment for prevention of a disease, condition, or disorder. In embodiments, the disease, condition, or disorder is cancer.

The term “polynucleotide” or “nucleic acid” refers to mRNA, RNA, cRNA, rRNA, cDNA or DNA. The term typically refers to a polymeric form of nucleotides of at least 10 bases in length, either ribonucleotides or deoxynucleotides or a modified form of either type of nucleotide. The term includes all forms of nucleic acids including single and double stranded forms of nucleic acids.

The terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms also encompass polynucleotides that are distinguished from a reference polynucleotide by the addition, deletion or substitution of at least one nucleotide. Accordingly, the terms “polynucleotide variant” and “variant” include polynucleotides in which one or more nucleotides have been added or deleted or replaced with different nucleotides. In this regard, it is well understood in the art that certain alterations inclusive of mutations, additions, deletions, and substitutions can be made to a reference polynucleotide whereby the altered polynucleotide retains the biological function or activity of the reference polynucleotide or has increased activity in relation to the reference polynucleotide (i.e., optimized). Polynucleotide variants include, for example, polynucleotides having at least 50% (and at least 51% to at least 99% and all integer percentages in between, e.g., 90%, 95%, or 98%) sequence identity with a reference polynucleotide sequence described herein. The terms “polynucleotide variant” and “variant” also include naturally-occurring allelic variants and orthologs.

The terms “polypeptide,” “polypeptide fragment,” “peptide,” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers. In certain aspects, polypeptides may include enzymatic polypeptides, or “enzymes,” which typically catalyze (i.e., increase the rate of) various chemical reactions.

The term “polypeptide variant” refers to polypeptides that are distinguished from a reference polypeptide sequence by the addition, deletion, or substitution of at least one amino acid residue. In embodiments, a polypeptide variant is distinguished from a reference polypeptide by one or more substitutions, which may be conservative or non-conservative. In embodiments, the polypeptide variant comprises conservative substitutions and, in this regard, it is well understood in the art that some amino acids may be changed to others with broadly similar properties without changing the nature of the activity of the polypeptide. Polypeptide variants also encompass polypeptides in which one or more amino acids have been added or deleted or replaced with different amino acid residues.

The term “promoter” refers to a DNA sequence recognized by the synthetic machinery of the cell or introduced synthetic machinery, required to initiate the specific transcription of a polynucleotide sequence. The term “expression control (regulatory) sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

The term “bind,” “binds,” or “interacts with” refers to a molecule recognizing and adhering to a particular second molecule in a sample or organism but does not substantially recognize or adhere to other structurally unrelated molecules in the sample. The term “specifically binds,” as used herein with respect to an antibody, refers to an antibody which recognizes a specific antigen, but does not substantially recognize or bind other molecules in a sample. For example, an antibody that specifically binds an antigen from one species may also bind that antigen from one or more species. But, such cross-species reactivity does not itself alter the classification of an antibody as specific. In another example, an antibody that specifically binds an antigen may also bind different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds a specific protein structure rather than to any protein. If an antibody is specific for epitope “A,” the presence of a molecule containing epitope A (or free, unlabeled A), in a reaction containing labeled “A” and the antibody, will reduce the amount of labeled A bound to the antibody.

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding, and protein-binding activity.

“Modulation” of gene expression refers to a change in the activity of a gene. Modulation of expression can include but is not limited to, gene activation and gene repression. Genome editing (e.g., cleavage, alteration, inactivation, random mutation) can be used to modulate expression. Gene inactivation refers to any reduction in gene expression as compared to a cell that does not include a ZFP as described herein. Thus, gene inactivation may be partial or complete.

A “region of interest” is any region of cellular chromatin, such as, for example, a gene or a non-coding sequence within or adjacent to a gene, in which it is desirable to bind an exogenous molecule. Binding can be for the purposes of targeted DNA cleavage and/or targeted recombination. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs.

By “statistically significant,” it is meant that the result was unlikely to have occurred by chance. Statistical significance can be determined by any method known in the art. Commonly used measures of significance include the p-value, which is the frequency or probability with which the observed event would occur if the null hypothesis were true. If the obtained p-value is smaller than the significance level, then the null hypothesis is rejected. In simple cases, the significance level is defined at a p-value of 0.05 or less. A “decreased” or “reduced” or “lesser” amount is typically a “statistically significant” or a physiologically significant amount, and may include a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.) an amount or level described herein.

The term “stimulation,” refers to a primary response induced by binding of a stimulatory molecule (e.g., a TCR/CD3 complex) with its cognate ligand thereby mediating a signal transduction event, such as signal transduction via the TCR/CD3 complex. Stimulation can mediate altered expression of certain molecules, such as downregulation of TGF-β, and/or reorganization of cytoskeletal structures. CD3 zeta is not the only suitable primary signaling domain for a CAR construct with respect to the primary response. For example, back in 1993, both CD3 zeta and FcR gamma were shown as functional primary signaling domains of CAR molecules. Eshhar et al., “Specific activation and targeting of cytotoxic lymphocytes through chimeric single chains consisting of antibody-binding domains and the gamma or zeta subunits of the immunoglobulin and T-cell receptors” PNAS, 1993 Jan. 15; 90(2):720-4, showed that two CAR constructs in which an scFv was fused to “either the FcRy chain or the CD3 complex s chain” triggered T-cell activation and target cell. Notably, as demonstrated in Eshhar et al., CAR constructs containing only the primary signaling domain CD3 zeta or FcR gamma are functional without the co-presence of co-stimulatory domains. Additional non-CD3 zeta based CAR constructs have been developed over the years. For example, Wang et al., “A Chimeric Antigen Receptor (CARs) Based Upon a Killer Immunoglobulin-Like Receptor (KIR) Triggers Robust Cytotoxic Activity in Solid Tumors” Molecular Therapy, vol. 22, no. Suppl.1, May 2014, page S57, tested a CAR molecule in which an scFv was fused to “the transmembrane and cytoplasmic domain of a killer immunoglobulin-like receptor (KIR). Wang et al. states that, “a KIR-based CAR targeting mesothelin (SS 1-KIR) triggers antigen-specific cytotoxic activity and cytokine production that is comparable to CD3˜-based CARs.” A second publication from the same group, Wang et al., “Generation of Potent T-cell Immunotherapy for Cancer Using DAP12-Based, Multichain, Chimeric Immunoreceptors” Cancer Immunol Res. 2015 July; 3(7):815-26, showed that a CAR molecule in which “a single-chain variable fragment for antigen recognition [was fused] to the transmembrane and cytoplasmic domains of KIR2DS2, a stimulatory killer immunoglobulin-like receptor (KIR)” functioned both in vitro and in vivo “when introduced into human T-cells with DAP12, an immunotyrosine-based activation motifs-containing adaptor.”

The term “stimulatory molecule” refers to a molecule on a T-cell that specifically binds a cognate stimulatory ligand present on an antigen presenting cell. For example, a functional signaling domain derived from a stimulatory molecule is the zeta chain associated with the T-cell receptor complex. The stimulatory molecule includes a domain responsible for signal transduction.

The term “stimulatory ligand” refers to a ligand that when present on an antigen presenting cell (e.g., an APC, a dendritic cell, a B-cell, and the like.) can specifically bind with a cognate binding partner (referred to herein as a “stimulatory molecule”) on a cell, for example a T-cell, thereby mediating a primary response by the T-cell, including activation, initiation of an immune response, proliferation, and similar processes. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “therapeutic” refers to a treatment and/or prophylaxis. A therapeutic effect is obtained by suppression, remission, or eradication of a disease state or alleviating the symptoms of a disease state.

The term “therapeutically effective amount” refers to the amount of the subject compound that will elicit the biological or medical response of a tissue, system, or subject that is being sought by the researcher, veterinarian, medical doctor or another clinician. The term “therapeutically effective amount” includes that amount of a compound that, when administered, is sufficient to prevent the development of, or alleviate to some extent, one or more of the signs or symptoms of the disorder or disease being treated. The therapeutically effective amount will vary depending on the compound, the disease and its severity and the age, weight, etc., of the subject to be treated.

The term “treat a disease” refers to the reduction of the frequency or severity of at least one sign or symptom of a disease or disorder experienced by a subject.

The term “transfected” or “transformed” or “transduced” refers to a process by which an exogenous nucleic acid is transferred or introduced into the host cell. A “transfected” or “transformed” or “transduced” cell is one which has been transfected, transformed, or transduced with exogenous nucleic acid. The cell includes the primary subject cell and its progeny.

The term “vector” refers to a polynucleotide that comprises an isolated nucleic acid and which can be used to deliver the isolated nucleic acid to the interior of a cell. Numerous vectors are known in the art including linear polynucleotides, polynucleotides associated with ionic or amphiphilic compounds, plasmids, and viruses. Thus, the term “vector” includes an autonomously replicating plasmid or a virus. The term also includes non-plasmid and non-viral compounds which facilitate transfer of nucleic acid into cells, such as, for example, polylysine compounds, liposomes, and the like. Examples of viral vectors include, adenoviral vectors, adeno-associated virus vectors, retroviral vectors, and others. For example, lentiviruses are complex retroviruses, which, in addition to the common retroviral genes gag, pol, and env, contain other genes with regulatory or structural function. Lentiviral vectors are well known in the art. Some examples of lentivirus include the Human Immunodeficiency Viruses: HIV-1, HIV-2, and the Simian Immunodeficiency Virus: SIV. Lentiviral vectors have been generated by multiply attenuating the HIV virulence genes, for example, the genes env, vif, vpr, vpu, and nef are deleted making the vector biologically safe.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Embodiments described herein relate to a method for increasing a number and/or ratio of a subpopulation of immune cells infused into a subject having cancer and/or increasing or extending persistence of a subpopulation of immune cells infused into the subject, the method comprising: administering an effective amount of a composition comprising T-cells to the subject, the T-cells including a chimeric antigen receptor (CAR) comprising an antigen binding molecule and a modified PD-1, the modified PD-1 lacking a functional PD-1 intracellular domain; and monitoring the T-cell response in the subject, wherein the subpopulation of immune cells comprising naive T-cells, stem cell memory T-cells, and/or central memory T-cells, and the number and/or ratio of the subpopulation of immune cells in the subject is increased as compared to that of a subject administered with corresponding T-cells that do not include the modified PD-1 or wherein the persistence of the subpopulation of immune cells in the subject is increased or extended as compared to that of a subject administered with the corresponding T-cells that do not include the modified PD-1.

As used herein, an immune cell refers to the cells of the immune system, and examples of immune cells include T cells, B cells, NK cells, neutrophils, and monocytes/macrophages. In embodiments, the immune cell refers to T cells.

In embodiments, the increase in the number of a subpopulation of immune cells includes an increase in the number of a subpopulation of CAR T-cells at a certain number of days (X days) after infusion or administering an effective amount of CAR T-cells including modified PD-1 to the subject. As an example, at day X of detection or measurement of immune cells including the subpopulation of immune cells, the number of the subpopulation of immune cells is higher than the subpopulation of immune cells initially infused or administered to the subject and/or the number of the subpopulation of immune cells is higher than the corresponding subpopulation of immune cells in a subject that is administered with the same amount of CAR T-cells without the modified PD-1.

In embodiments, the increase in the ratio of a subpopulation of immune cells includes an increase in the ratio of a subpopulation of CAR T-cells at a certain number of days (X days) after infusion or administering an effective amount of CAR T-cells including modified PD-1 to the subject. In embodiments, the ratio includes the number of the subpopulation of CAR T-cells in the subject over the total number of CAR T-cells in the subject. The ratio can also be the percent of the subpopulation of CAR T-cells, for example, the percent of the subpopulation of CAR T-cells is the number of the subpopulation of CAR T-cells infused or administered to the subject as compared to (over) the total number of CAR T-cells infused or administered to the subject times 100 (% of subpopulation of CAR T-cells=(number of subpopulation of CAR T-cells/number of CAR T-cells infused or administered to the subject)×100. As an example, at day X of detection or measurement of immune cells including the subpopulation of immune cells, the ratio of the subpopulation of CAR T-cells is higher than the ratio of subpopulation of immune cells initially infused or administered to the subject and/or the ratio of the subpopulation of immune cells is higher than the ratio of the corresponding subpopulation of immune cells in a subject that is administered with the same amount of CAR T-cells without the modified PD-1.

In embodiments, a subpopulation of immune cells includes central memory T-cells (Tcm cells). In embodiments, the X number of days after infusion or administering the effective amount of T-cells includes a range of 5 to 40 days. The X number of days after infusion or administering includes 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days, wherein day 0 is the day of infusion.

In embodiments, the subpopulation of immune cells may have improved expansion and viability; and/or improved ability in tumor clearance and enhanced persistence in response to tumor challenges. Tumor challenges include culturing agents with tumor cells to determine whether the agent kills or inhibits the growth of tumor cells. As an example, determining includes measuring the number of tumor cells left after the tumor challenge to determine whether the agent can kill the tumor cells. The agents include CAR T-cells and CAR T-cells including modified PD-1. In embodiments, the subpopulation of immune cells may have increased gene expression in at least one of CD27, CCR7, and CD62L; decreased gene expression in at least one of PD-1 and Tim-3; increased central memory T-cell subpopulation; and/or decreased effector T-cell subpopulation as compared to T-cells that do not include the modified PD-1.

In embodiments, monitoring the T-cell response in the subject comprises detecting or measuring at least one of: mRNA of the modified PD-1; the number of white blood cells; the number of naive T-cells, stem cell memory T-cells, and central memory T-cells; the CAR copy number; the number of CD3 positive cell; the number of T-cells expressing CAR; or the level of one or more cytokines. In embodiments, monitoring the T-cell response in the subject comprises monitoring the T-cell response in peripheral blood, bone marrow, lymph node tissue, cord blood, thymus tissue, tissue from a site of infection, ascites, pleural effusion, spleen tissue, or tumors of the subject. For example, monitoring the T-cell response in the subject comprises monitoring the T-cell response in peripheral blood of the subject.

As used herein, the terms “T lymphocyte” and “T-cell” are used interchangeably and refer to a principal type of white blood cell that completes maturation in the thymus and that has various roles in the immune system, including the identification of specific foreign antigens in the body and the activation and deactivation of other immune cells. A T-cell can be any T-cell, such as a cultured T-cell, for example, a primary T-cell a T-cell from a cultured T-cell line (e.g., Jurkat, SupT 1, etc.), or a T-cell obtained from a mammal. The T-cell can be CD3+ cells. The T-cell can be any type of T-cell and can be of any developmental stage, including but not limited to, CD4+/CD8+ double positive T-cells, CD4+ helper T-cells (e.g., Th1 and Th2 cells), CD8+ T-cells (e.g., cytotoxic T-cells), a T-cell present among peripheral blood mononuclear cells (PBMCs), peripheral blood leukocytes (PBLs), a T-cell that is among tumor infiltrating lymphocytes (TILs), memory T-cells, naive T-cells, regulatory T-cells, gamma delta T-cells (76 T-cells). Additional types of helper T-cells include cells such as Th3, Th17, Th9, or Tfh cells. Additional types of memory T-cells include cells such as central memory T-cells (Tcm cells), effector memory T-cells (Tem cells and TEIVIRA cells). The T-cell can also refer to a genetically engineered T-cell, such as a T-cell modified to express a T-cell receptor (TCR) or a chimeric antigen receptor (CAR). The T-cell can also be differentiated from a stem cell or progenitor cell.

As used herein, the term “naive T-cell” or Tn, refers to mature T-cells that, unlike activated or memory T-cells, have not encountered their cognate antigen within the periphery. Naive T-cells are commonly characterized by the surface expression of L-selectin (CD62L); the absence of the activation markers CD25, CD44 or CD69; and the absence of the memory CD45RO isoform. They also express functional IL-7 receptors, consisting of subunits IL-7 receptor-u, CD127, and common-7 chain, and CD132. In the naive state, T-cells are thought to be quiescent and non-dividing, requiring the common gamma chain cytokines IL-7 and IL-15 for homeostatic survival mechanisms.

As used herein, the term “central memory T-cells” or Tcm, refers to a subgroup or subpopulation of T-cells that express CD45RO and CD25 but do not express CD45RA. Tcm cells also press genes associated with trafficking to secondary lymphoid organs, including CD62L, CXCR3, CCR7, in contrast to effector memory T-cells or Tem cells (effector memory T cells) that lose expression of these gene products.

As used herein, the term ‘stem memory T-cells,” or “stem cell memory T-cells”, or Tscm, refers to a subgroup or subpopulation of T-cells that are capable of self-renewing and generating Tcm. Tem and Teff(effector T-cells) Tscm have an expression pattern similar to Tn but unlike Tn, they also express CD95.

As used herein, the term “population” when used with reference to T-cells refers to a group of cells including two or more T-cells, respectively. Using T-cell as an example, the isolated, or enriched, population of T-cells may include only one type of T-cell or may include a mixture of two or more types of T-cell. The isolated population of T-cells can be a homogeneous population of one type of T-cell or a heterogeneous population of two or more types of T-cell. The isolated population of T-cells can also be a heterogeneous population having T-cells and at least a cell other than a T-cell, e.g., a B cell, a macrophage, a neutrophil, an erythrocyte, a hepatocyte, an endothelial cell, an epithelial cell, a muscle cell, a brain cell, etc. The heterogeneous population can have from 0.01% to about 100% T-cell. Accordingly, an isolated population of T-cells can have at least 50%, 60% 70%, 80%, 90%, 95%, 98%, 99% T-cells. The isolated population of T-cells can include one or more, or all of, the different types of T-cells, including but not limited to those disclosed herein. In an isolated population of T-cells that includes more than one type of T-cells, the relative ratio of each type of T-cell can range from 0.01% to 99.99% The isolated population also can be a clonal population of T-cells, in which all the T-cells of the population are clones of a single T-cell.

An isolated population of T-cells can be obtained from a natural source, such as human peripheral blood or cord blood. Various ways of dissociating cells from tissues or cell mixtures to separate the various cell types have been developed in the art. These manipulations can result in a relatively homogeneous population of cells. The T-cells can be isolated by a sorting or selection process as described herein or by other methods known in the art. The proportion of T-cells in the isolated population can be higher than the proportion of T-cells in the natural source by at least about 10%, about 20%, about 30%, about 40%, about 50%, about 60%, about 70%, about 80%, about 85%, about 90%, or about 95%. The isolated population of T-cells can be T-cells, or one or more specific types of T-cells. As used herein, the term “subpopulation” when used in reference to T-cells refers to a population of T-cells that includes less than all types of T-cells, respectively, that are found in nature.

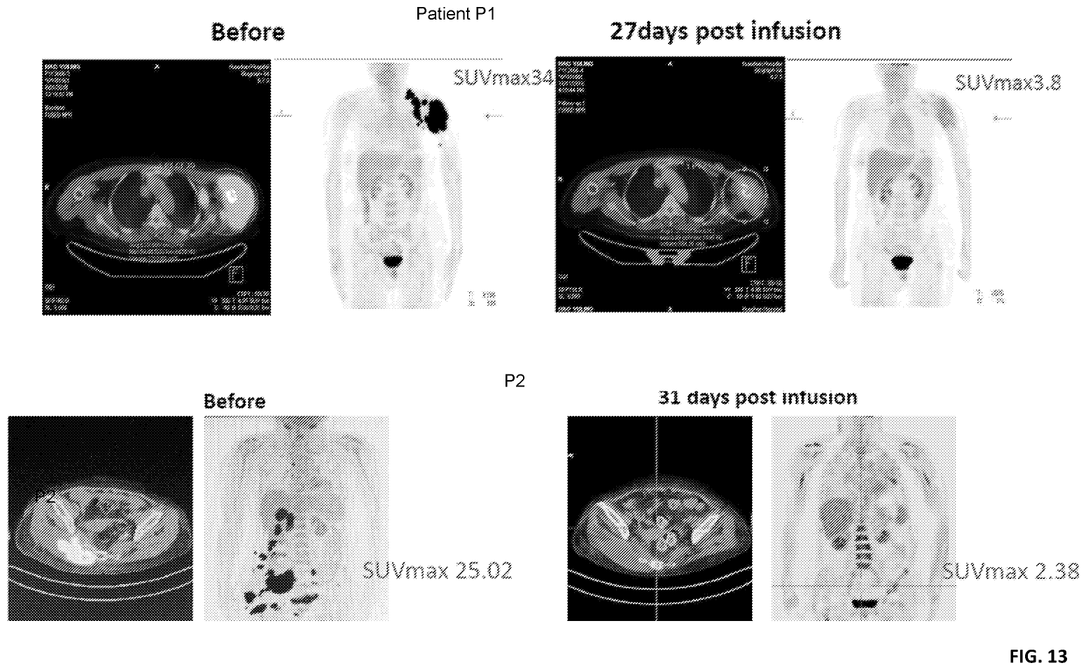

In embodiments, the method described herein comprises monitoring phenotypes and other parameters (e.g., FIGS. 13 and 15-18) of CAR T-cells after the administration of the T-cells. In embodiments, the population of modified T-cells include more memory T-cells in the subject than the T-cells of a subject introduced with the CAR but without the modified PD-1. The amount of memory T-cells is increased in the subject administered with the CAR and dnPD-1 as compared to the amount of T-cells in a subject administered with the CAR without the modified PD-1. In embodiments, the modified PD-1 comprises an amino acid sequences of SEQ ID: 36, 37, 38, 39 or 40 or the modified PD-1 does not include SEQ ID: 41 and 42. In embodiments, the T-cells comprise a dominant negative variant of PD-1.

In embodiments, the CAR comprises an extracellular domain, a transmembrane domain, and an intracellular domain, the extracellular domain binds an antigen. In embodiments, the extracellular domain comprises one or more of SEQ ID: 5-17 or 30-35. In embodiments, the T-cells comprise one of SEQ ID: 18-29. In embodiments, the intracellular domain comprises a co-stimulatory signaling region that comprises an intracellular domain of a co-stimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, and any combination thereof. In embodiments, the antigen is Epidermal growth factor receptor (EGFR), Variant III of the epidermal growth factor receptor (EGFRvIII), Human epidermal growth factor receptor 2 (HER2), Mesothelin (MSLN), Prostate-specific membrane antigen (PSMA), Carcinoembryonic antigen (CEA), Disialoganglioside 2 (GD2), Interleukin-13Ra2 (IL13Ra2), Glypican-3 (GPC3), Carbonic anhydrase IX (CAIX), L1 cell adhesion molecule (L1-CAM), Cancer antigen 125 (CA125), Cluster of differentiation 133 (CD133), Fibroblast activation protein (FAP), Cancer/testis antigen 1B (CTAG1B), Mucin 1 (MUC1), Folate receptor-α (FR-α), CD19, FZD10, TSHR, PRLR, Muc 17, GUCY2C, CD207, CD3, CD5, B-Cell Maturation Antigen (BCMA), or CD4.

In embodiments, the T-cells comprise a CAR binding to a solid tumor antigen and a CAR binding a white cell antigen. In embodiments, the solid tumor antigen is tMUC 1, PRLR, CLCA1, MUC12, GUCY2C, GPR35, CR1L, MUC 17, TMPRSS11B, MUC21, TMPRSS11E, CD207, SLC30A8, CFC1, SLC12A3, SSTR1, GPR27, FZD10, TSHR, SIGLEC15, SLC6A3, KISS1R, QRFPR, GPR119, CLDN6, UPK2, ADAM12, SLC45A3, ACPP, MUC21, MUC16, MS4A12, ALPP, CEA, EphA2, FAP, GPC3, IL13-Ra2, Mesothelin, PSMA, ROR1, VEGFR-II, GD2, FR-α, ErbB2, EpCAM, EGFRvIII, or EGFR. In embodiments, the white cell antigen is a B cell antigen. In embodiments, the B cell antigen is CD19, CD20, CD22, or BCMA.

Embodiments relate to a pharmaceutical composition comprising human T-cells, wherein the human T-cells comprise a first nucleic acid sequence that encodes truncated CTLA4 that reduces an inhibitory effect of CTLA4 ligand 1 on the human T-cells, the truncated CTLA4 lacking a functional CTLA4 intracellular domain; and a second nucleic acid sequence that encodes a chimeric antigen receptor (CAR) comprising an extracellular domain that recognizes a tumor antigen of the target cell, a transmembrane domain, and an intracellular domain comprising a CD3-zeta signaling domain and a signaling domain of a co-stimulatory molecule, and wherein the truncated CTLA4 and the CAR are expressed as gene products that are separate polypeptides. Embodiments relate to population of CAR cells comprising the first and second nucleic acid sequences. Embodiments relate a pharmaceutical composition comprising the population of the CAR T-cells. Embodiments relate a method of inducing T-cell response in a subject in need thereof and/or treating a tumor of the subject, the method comprising administering an effective amount of the composition to the subject.

In embodiments, the truncated CTLA4 interferes with a pathway between CTLA4 of a human T-cell and CTLA4 ligand of a target cell. In embodiments, the truncated CTLA4 comprises a CTLA4 extracellular domain or a CTLA4 transmembrane domain, or a combination thereof. In embodiments, the truncated CTLA4 comprises one of the amino acid sequences of SEQ ID: 43-51.

Embodiments relate to a method for increasing a number and/or ratio of a subpopulation of immune cells infused into a subject and/or increasing or extending persistence of a subpopulation of immune cells infused into a subject, the method comprising: introducing a population of T-cells with a nucleic acid sequence encoding a chimeric antigen receptor (CAR); and introducing the population of T-cells with a nucleic acid sequence encoding a modified PD-1, the modified PD-1 lacking a functional PD-1 intracellular domain, wherein the subpopulation of immune cells comprising naive T-cells, stem cell memory T-cells, and/or central memory T-cells, and the number and/or ratio of the subpopulation of immune cells is increased in the population of T-cells as compared to corresponding population of T-cells that do not include the modified PD-1 or the persistence of the subpopulation of immune cells is increased or extended in the population of T-cells as compared to the corresponding population of T-cells that do not include the modified PD-1.

In embodiments, the antigen binding molecule is a chimeric antigen receptor (CAR) or a T-cell Receptor (TCR). The term “Chimeric Antigen Receptor” or alternatively a “CAR” refers to a recombinant polypeptide construct comprising at least an extracellular antigen binding domain, a transmembrane domain, and an intracellular domain (e.g., cytoplasmic domain). The intracellular domain can include a signaling domain. In embodiments, the domains in the CAR polypeptide construct are on the same polypeptide chain (e.g., a chimeric fusion protein) or are not contiguous with each other (e.g., on different polypeptide chains).

In embodiments, the intracellular domain includes a functional signaling domain derived from a stimulatory molecule and/or a co-stimulatory molecule as described above. In embodiments, the intracellular domain includes a functional signaling domain derived from a primary signaling domain (e.g., a primary signaling domain of CD3-zeta). In embodiments, the intracellular signaling domain further includes one or more functional signaling domains derived from at least one co-stimulatory molecule. The co-stimulatory signaling region refers to a portion of the CAR including the intracellular domain of a co-stimulatory molecule. Co-stimulatory molecules are cell surface molecules other than antigen receptors or they are molecules in which their ligands are required for an efficient response of lymphocytes to antigen.

Between the extracellular domain and the transmembrane domain of the CAR, there may be incorporated a spacer domain. As used herein, the term “spacer domain” generally means any oligo- or polypeptide that functions to link the transmembrane domain to either the extracellular domain or the cytoplasmic domain in the polypeptide chain. A spacer domain may include up to 300 amino acids, 10 to 100 amino acids, or 25 to 50 amino acids.

The extracellular domain of a CAR may include an antigen binding domain, for example, an scFv, a single domain antibody, or TCR (e.g., a TCR alpha binding domain or TCR beta binding domain), that targets a specific tumor marker (e.g., a tumor antigen). Tumor antigens are proteins that are produced by tumor cells that elicit an immune response, particularly T-cell mediated immune responses. Tumor antigens are well known in the art and include, for example, a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin. As an example, the tumor antigen is CD19, and the CAR thereof is referred to as CD19CAR.

In embodiments, the extracellular ligand-binding domain comprises an scFv comprising the light chain variable (VL) region and the heavy chain variable (VH) region of a target antigen-specific monoclonal antibody joined by a flexible linker. Single chain variable region fragments are made by linking light and/or heavy chain variable regions by using a short linking peptide (Bird et al., Science 242:423-426, 1988). An example of a linking peptide is the GS linker having the amino acid sequence (GGGGS)3 (SEQ ID NO: 60), which bridges approximately 3.5 nm between the carboxy terminus of one variable region and the amino terminus of the other variable region. Linkers of other sequences have been designed and used (Bird et al., 1988, supra). Linkers can be short, flexible polypeptides and comprising about 20 or fewer amino acid residues. Linkers can, in turn, be modified for additional functions, such as attachment of drugs or attachment to solid supports. The single chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, such as a eukaryotic cell, such as yeast, plant, insect, or mammalian cells, or a prokaryotic cell, such as E. coli. Polynucleotides encoding the scFv of interest can be made by routine manipulations such as ligation of polynucleotides. The resultant scFv can be isolated using standard protein purification techniques known in the art.

In embodiments, the tumor antigen includes HER2, CD19, CD20, CD22, Kappa or light chain, CD30, CD33, CD123, CD38, ROR1, ErbB3/4, EGFR, EGFRvIII, EphA2, FAP, carcinoembryonic antigen, EGP2, EGP40, mesothelin, TAG72, PSMA, NKG2D ligands, B7-H6, IL-13 receptor α2, IL-11 receptor α, MUC1, MUC16, CA9, GD2, GD3, HMW-MAA, CD171, Lewis Y, G250/CAIX, HLA-A1 MAGE A1, HLA-A2 NY-ESO-1, PSC1, folate receptor-α, CD44v7/8, 8H9, NCAM, VEGF receptors, 5T4, Fetal AchR, NKG2D ligands, CD44v6, TEM1, TEM8, or viral-associated antigens expressed by the tumor.

In embodiments, the antigen binding molecule is a T-cell Receptor (TCR). In embodiments, the TCR is modified TCR. In embodiments, the TCR is derived from spontaneously occurring tumor-specific T-cells in patients. In embodiments, the TCR binds to a tumor antigen. In embodiments, the tumor antigen comprises CEA, gp100, MART-1, p53, MAGE-A3, or NY-ESO-1. In embodiments, the TCR comprises TCRγ and TCRδ Chains or TCRα and TCRβ chains. In embodiments, a T-cell clone that expresses a TCR with high affinity for the target antigen can be isolated. In embodiments, tumor-infiltrating lymphocytes (TILs) or peripheral blood mononuclear cells (PBMCs) can be cultured in the presence of antigen-presenting cells (APCs) pulsed with a peptide representing an epitope known to elicit a dominant T-cell response when presented in the context of a defined HLA allele. High-affinity clones can then be selected on the basis of MHC-peptide tetramer staining and/or the ability to recognize and lyse target cells pulsed with low titrated concentrations of cognate peptide antigen. After the clone has been selected, the TCRα and TCRβ chains or TCRγ and TCRδ Chains are identified and isolated by molecular cloning. For example, for TCRα and TCRβ chains, the TCRα and TCRβ gene sequences are then used to generate an expression construct that ideally promotes stable, high-level expression of both TCR chains in human T-cells. The transduction vehicle (e.g., a gammaretrovirus or lentivirus) can then be generated and tested for functionality (antigen specificity and functional avidity) and used to produce a clinical lot of the vector. An aliquot of the final product can then be used to transduce the target T-cell population (generally purified from patient PBMCs), which is expanded before infusion into the patient.

In embodiments, the binding element of the CAR may include any antigen binding moiety that when bound to its cognate antigen, affects a tumor cell, such as inhibiting the growth of the tumor cell or kills the tumor cell.

The nucleic acid sequences encoding the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The embodiments of the present disclosure further relate to vectors in which an encoding a molecule described herein such as the CAR or dnPD-1 is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

Viruses can be used to deliver nucleic acids into a cell in vitro and in vivo (in a subject). Examples of viruses useful for delivery of nucleic acids into cells include retrovirus, adenovirus, herpes simplex virus, vaccinia virus, and adeno-associated virus.

There also exist non-viral methods for delivering nucleic acids into a cell, for example, electroporation, gene gun, sonoporation, magnetofection, and the use of oligonucleotides, lipoplexes, dendrimers, and inorganic nanoparticles.

The expression of natural or synthetic nucleic acids encoding CARs is typically achieved by operably linking a nucleic acid encoding the CAR polypeptide or portions thereof to one or more promoters and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

Additional information related to expression synthetic nucleic acids encoding CARs and gene transfer into mammalian cells is provided in U.S. Pat. No. 8,906,682, incorporated by reference in its entirety.

The present disclosure describes compositions and pharmaceutical composition including T-cells described herein. Pharmaceutical compositions of the present disclosure can be administered in a manner appropriate to the disease to be treated (or prevented). The quantity and frequency of administration will be determined by such factors as the condition of the patient, and the type and severity of the patient's disease, although appropriate dosages may be determined by clinical trials.

When “an immunologically effective amount”, “an anti-tumor effective amount”, “a tumor-inhibiting effective amount”, or “therapeutic amount” is indicated, the precise amount of the compositions of the present disclosure to be administered can be determined by a physician with consideration of individual differences in age, weight, tumor size, extent of infection or metastasis, and condition of the patient (subject). It can be stated that a pharmaceutical composition comprising the T-cells described herein can be administered at a dosage of 104 to 109 cells/kg body weight, \105 to 106 cells/kg body weight, including all integer values within those ranges. T-cell compositions can also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly. In embodiments, it may be desired to administer activated T-cells to a subject and then subsequently redraw the blood (or have apheresis performed), collect the activated and expanded T-cells, and reinfuse the patient with these activated and expanded T-cells. This process can be carried out multiple times every few weeks. In embodiments, T-cells can be activated from blood draws of from 10 cc to 400 cc. In embodiments, T-cells are be activated from blood draws of 20 cc, 30 cc, 40 cc, 50 cc, 60 cc, 70 cc, 80 cc, 90 cc, or 100 cc. Not to be bound by theory, using multiple blood draw/multiple reinfusion protocols, can select out certain populations of T-cells.

The administration of the pharmaceutical compositions described herein can be carried out in any convenient manner, including by aerosol inhalation, injection, ingestion, transfusion, implantation or transplantation. The compositions described herein can be administered to a patient subcutaneously, intradermally, intratumorally, intranodally, intramedullary, intramuscularly, by intravenous (i. v.) injection, or intraperitoneally. In embodiments, the T-cell compositions described herein are administered to a patient by intradermal or subcutaneous injection. In embodiments, the T-cell compositions of the present disclosure are administered by i.v. injection. The compositions of T-cells described herein can be injected directly into a tumor, lymph node, or site of infection. In embodiments of the present disclosure, cells activated and expanded using the methods described herein, or other methods known in the art in which T-cells are expanded to therapeutic levels, are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or natalizumab treatment for MS patients or efalizumab treatment for psoriasis patients or other treatments for PML patients. In further embodiments, the T-cells of the present disclosure can be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytotoxin, fludaribine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin). (Liu et al., Cell 66:807-815, 1991; Henderson et al., Immun 73:316-321, 1991; Bierer et al., Curr. Opin. Immun 5:763-773, 1993; Isoniemi (supra)). In embodiments, the T-cell compositions described herein are administered to a patient in conjunction with (e.g., before, simultaneously or following) bone marrow transplantation, T-cell ablative therapy using either chemotherapy agents such as, fludarabine, external-beam radiation therapy (XRT), cyclophosphamide, or antibodies such as OKT3 or CAMPATH. In embodiments, the cell compositions described herein are administered following B-cell ablative therapy such as agents that react with CD20, e.g., Rituxan. For example, subjects can undergo standard treatment with high dose chemotherapy followed by peripheral blood stem cell transplantation. In embodiments, following transplant, subjects receive an infusion of expanded immune cells described herein. In embodiments, expanded cells are administered before or following surgery.

The dosage of the above treatments to be administered to a patient will vary with the precise nature of the condition being treated and the recipient of the treatment. The scaling of dosages for human administration can be performed according to art-accepted practices by a physician depending on various factors.

Additional information on the methods of cancer treatment using engineered or modified T-cells is provided in U.S. Pat. No. 8,906,682, incorporated by reference in its entirety.

Embodiments relate to an in vitro method for preparing modified cells. The method includes obtaining a sample of cells from the subject. For example, the sample may include T-cells or T-cell progenitors. The method may further include transfecting the cells with a DNA encoding at least a CAR, culturing the population of CAR cells ex vivo in a medium that selectively enhances proliferation of CAR-expressing T-cells.

In embodiments, the sample can be a cryopreserved sample. In embodiments, the sample of cells is from umbilical cord blood or a peripheral blood sample from the subject. In embodiments, the sample of cells is obtained by apheresis or venipuncture. In embodiments, the sample of cells is a subpopulation of T-cells.

Embodiments also relate to a modified cell comprising a plurality of nucleic acid sequences encoding a first antigen binding molecule, a second antigen binding molecule, and a dominant negative form of an immune checkpoint molecule. Embodiments relate to a modified cell comprising a first binding molecule, a second binding molecule, and a dominant negative form of an immune checkpoint molecule. Embodiments relate to a method of preparing modified cells, the method comprising: introducing a plurality of nucleic acid sequences into cells to generate modified cells. Embodiments relate to a method of inducing or causing T-cell response, including treating a subject having cancer by enhancing cellular treatment on the subject having cancer, or inhibiting growth of tumor cells, the method comprising: introducing into a cell a plurality of nucleic acid sequences encoding a first antigen binding molecule, a second antigen binding molecule, and a dominant negative form of an immune checkpoint molecule.

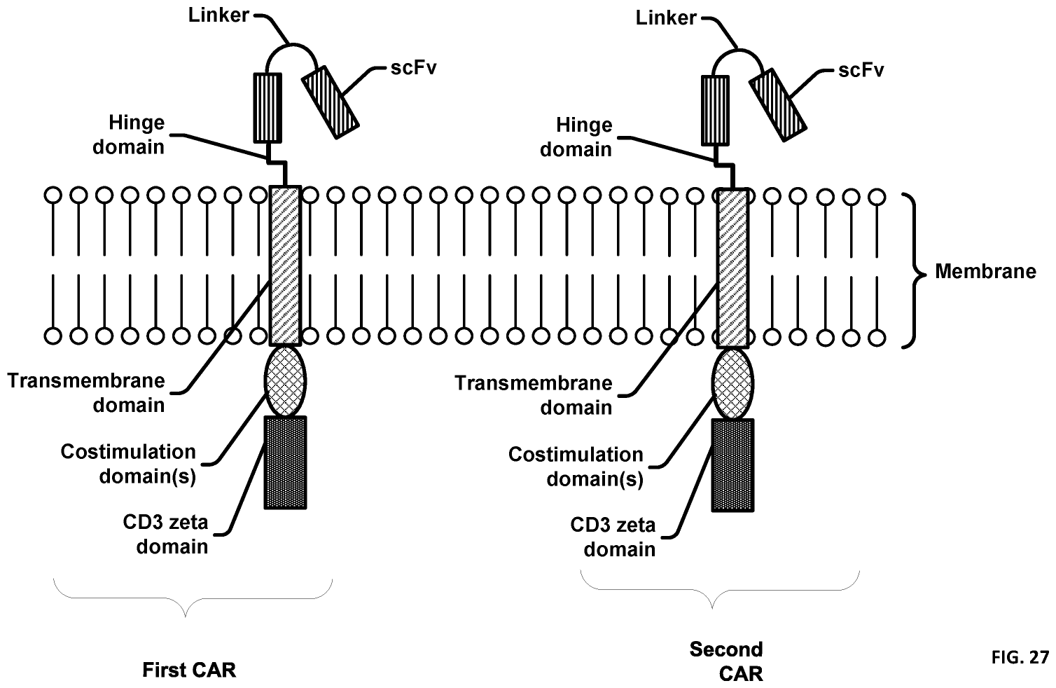

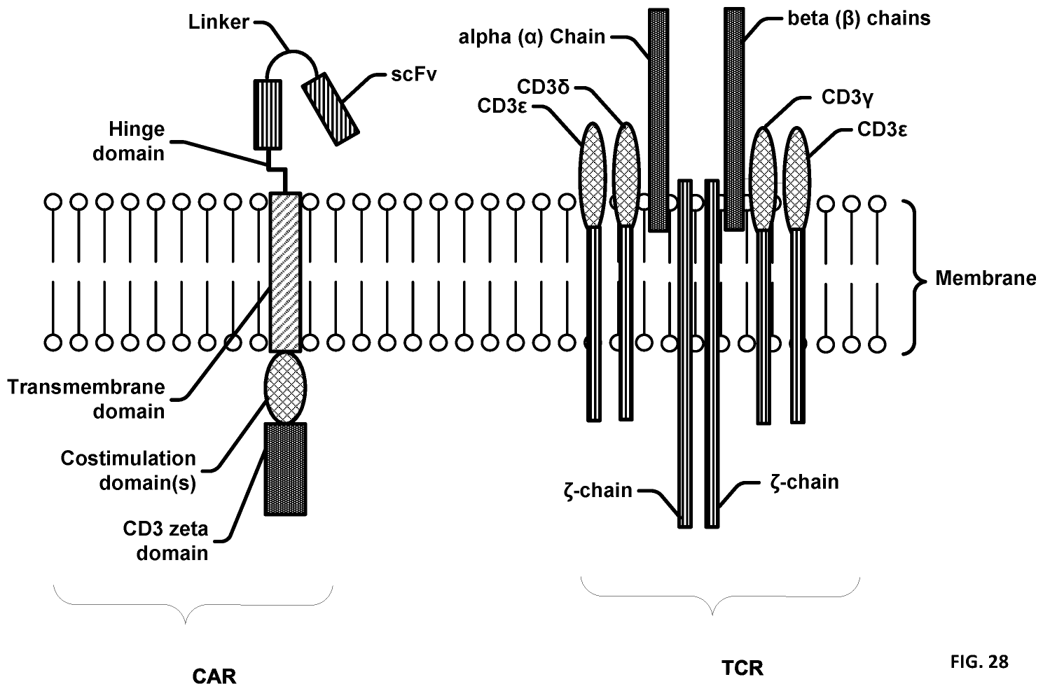

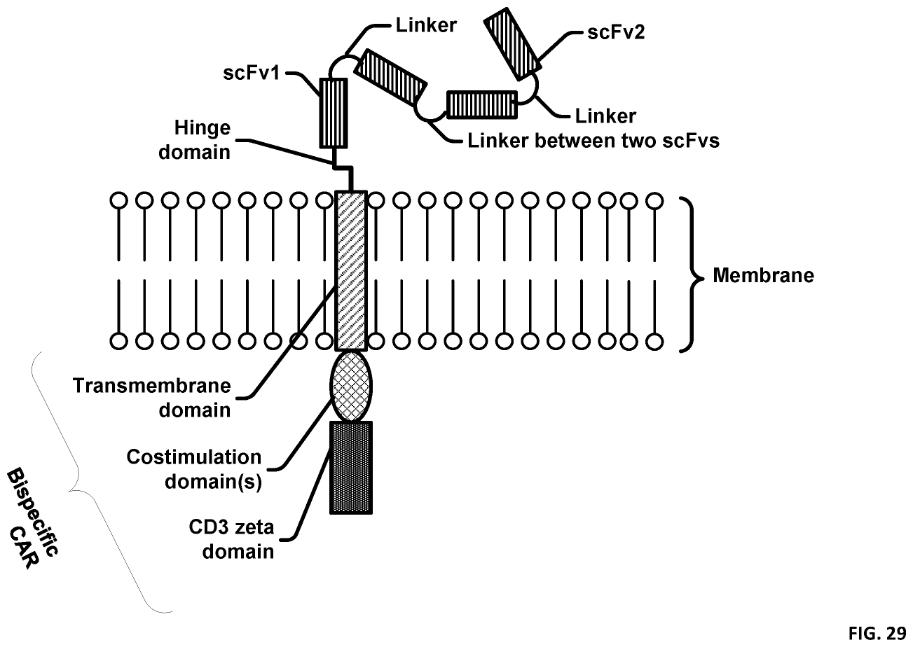

An antigen binding molecule refers to a molecule and/or domain binding a target molecule. In embodiments, the antigen binding molecule comprises a CAR or a TCR (See FIGS. 27 and 28). In embodiments, the antigen binding molecule comprises or a binding domain of a bispecific CAR, which includes two binding antigen molecules/domains (See FIG. 29).

An immune checkpoint molecule refers to a molecule that is associated with the T-cells and regulates T-cell response. In embodiments, the immune checkpoint molecule is selected from the group consisting of programmed death 1 (PD-1), cytotoxic T lymphocyte antigen-4 (CTLA-4), B- and T-lymphocyte attenuator (BTLA), T-cell immunoglobulin mucin-3 (TIM-3), lymphocyte-activation protein 3 (LAG-3), T-cell immunoreceptor with Ig and ITIM domains (TIGIT), leukocyte-associated immunoglobulin-like receptor 1 (LAIRI), natural killer cell receptor 2B4 (2B4), and CD 160.

Dominant negative mutations have an altered gene product that acts antagonistically to the wild-type allele. These mutations usually result in an altered molecular function (often inactive) and are characterized by a dominant or semi-dominant phenotype. In embodiments, an immune checkpoint molecule is a dominant negative variant of the immune checkpoint molecule and is capable of blocking a functional signaling pathway of the immune checkpoint. In embodiments, the immune checkpoint molecule is a receptor of T-cells. For example, the dominant negative form of the receptor may include one or more additions, deletions, or substitutions of the wide-type intracellular domain of the receptor such that a signaling pathway of the receptor may be blocked.

In embodiments, the first binding molecule, the second binding molecule, and the dominant negative of the immune checkpoint molecule are expressed as gene products that are separate polypeptides. In embodiments, the first binding molecule is a chimeric antigen receptor (CAR) binding CD19, the second binding molecule is a CAR binding tMUC1, and the dominant negative form of the immune checkpoint molecule is modified PD-1 lacking a functional PD-1 intracellular domain for PD-1/PD-L1 signal transduction. In embodiments, the modified cell comprises a bispecific chimeric antigen receptor (CAR) comprising the first binding molecule and the second binding molecule, wherein the bispecific CAR and the dominant negative of the immune checkpoint molecule are expressed as gene products that are separate polypeptides. In embodiments, the first antigen binding molecule is a first CAR.

In embodiments, the first antigen binding molecule binds a molecule associated with the blood of the subject. In embodiments, the first antigen binding molecule binds a surface molecule of a blood cell or a white blood cell. In embodiments, the first antigen binding molecule binds an antigen of a white blood cell (WBC). In embodiments, the antigen of white blood cell comprises CD19, CD22, CD20, BCMA, CD5, CD7, CD2, CD16, CD56, CD30, CD14, CD68, CD11b, CD18, CD169, CD1c, CD33, CD38, CD138, CD13, B7, CAIX, CD123, CD133, CD171, CD171/L1-CAM, CEA, Claudin 18.2, cMet, CS1, CSPG4, Dectin1, EGFR, EGFR vIII, EphA2, ERBB receptors, ErbB T4, ERBB2, FAP, Folate receptor 1, FITC, Folate receptor 1, FSH, GD2, GPC3, HA-1 H/HLA-A2, HER2, IL-11Ra, IL13 receptor a2, IL13R, IL13Ra2 (zetakine), Kappa, Leukemia, LewisY, Mesothelin, MUC1, NKG2D, NY-ESO-1, PSMA, ROR-1, TRAIL-receptor1, or VEGFR2. In embodiments, the WBC is a granulocyte, a monocyte and or lymphocyte. In embodiments, the WBC is a B cell. In embodiments, the cell surface molecule of the WBC is CD19.

Blood cells comprises red blood cells (RBCs), white blood cells (WBCs), platelets, or other blood cells. For example, RBCs are blood cells for delivering oxygen (02) to the body tissues via the blood flow through the circulatory system. Platelets are cells that are involved in hemostasis, leading to the formation of blood clots. WBCs are cells of the immune system involved in defending the body against both infectious disease and foreign materials. There are a number of different types and sub-types of WBCs and each has a different role to play. For example, granulocytes, monocytes, and lymphocytes are 3 major types of white blood cell. There are three different forms of granulocytes: Neutrophils, Eosinophils, Basophils.

A cell surface molecule of a WBC refers to a molecule expressed on the surface of the WBC. For example, the cell surface molecule of a lymphocyte includes CD19, CD22, CD20, BCMA, CD5, CD7, CD2, CD16, CD56, and CD30. The cell surface molecule of a B cell includes CD19, CD20, CD22, and BCMA. The cell surface molecule of a monocyte includes CD14, CD68, CD11b, CD18, CD169, and CD1c. The cell surface molecule of granulocyte includes CD33, CD38, CD138, and CD13.

In embodiments, the first binding molecule binds CD19, CD22, CD20, BCMA, CD5, CD7, CD2, CD16, CD56, CD30, CD14, CD68, CD11b, CD18, CD169, CD1c, CD33, CD38, CD138, or CD13, or the first binding molecules binds the cell surface molecule of a WBC, such as CD19, CD20, CD22, or BCMA.

In embodiments, the second binding molecule and the first binding molecule bind to different antigens. In embodiments, the second binding molecule binds a solid tumor antigen. In embodiments, the solid tumor antigen comprises MUC 1, PRLR, CLCA1, MUC12, GUCY2C, GPR35, CR1L, MUC 17, TMPRSS11B, MUC21, TMPRSS11E, CD207, SLC30A8, CFC1, SLC12A3, SSTR1, GPR27, FZD10, TSHR, SIGLEC15, SLC6A3, KISS1R, QRFPR, GPR119, CLDN6, UPK2, ADAM12, SLC45A3, ACPP, MUC21, MUC16, MS4A12, ALPP, CEA, EphA2, FAP, GPC3, IL13-Ra2, Mesothelin, PSMA, ROR1, VEGFR-II, GD2, FR-α, ErbB2, EpCAM, EGFRvIII, or EGFR. In embodiments, the solid tumor antigen comprisesB7, CAIX, CD123, CD133, CD171, CD171/L1-CAM, CEA, Claudin 18.2, cMet, CS1, CSPG4, Dectin1, EGFR, EGFR vIII, EphA2, ERBB receptors, ErbB T4, ERBB2, FAP, Folate receptor 1, FITC, Folate receptor 1, FSH, GD2, GPC3, HA-1 H/HLA-A2, HER2, IL-11Ra, IL13 receptor a2, IL13R, IL13Rα2 (zetakine), Kappa, Leukemia, LewisY, Mesothelin, MUC1, NKG2D, NY-ESO-1, PSMA, ROR-1, TRAIL-receptor1, or VEGFR2. In embodiments, the MUC1 is a tumor form of human MUC1 (tMUC1). In embodiments, the MUC1 is a tumor-exclusive epitope of a human MUC1, and the first CAR and the second CAR are expressed as polypeptides.

In embodiments, the MUC1 is a tumor-exclusive epitope of a human MUC1, and the first CAR and the second CAR or the TCR are expressed as separate polypeptides.

In embodiments, the MUC1 is a tumor form of human MUC1 (tMUC1). In embodiments, the first CAR includes a co-stimulatory domain without a signaling domain, such as the CD3 zeta domain, and the MUC1CAR (second CAR) comprises the MUC1 binding domain, a transmembrane domain, a co-stimulatory, and a CD3 zeta domain.

As used herein, the term “MUC1” refers to a molecule defined as follows. MUC1 is one of the epithelial mucin family of molecules. MUC1 is a transmembrane mucin glycoprotein that is normally expressed on all glandular epithelial cells of the major organs. In normal cells, MUC1 is only expressed on the apical surface and is heavily glycosylated with its core proteins sequestered by the carbohydrates. As cells transform to a malignant phenotype, expression of MUC1 increases several folds, and the expression is no longer restricted to the apical surface, but it is found all around the cell surface and in the cytoplasm. In addition, the glycosylation of tumor associated MUC1 is aberrant, with greater exposure of the peptide core than is found on MUC1 expressed in normal tissues. Little is known regarding the specifics of the aberrant glycosylation.

MUC1 is widely expressed on a large number of epithelial cancers and is aberrantly glycosylated making it structurally and antigenically distinct from that expressed by non-malignant cells (see, e.g., Barratt-Boyes, 1996; Price et al., 1998; Peterson et al., 1991). The dominant form of MUC1 is a high molecular weight molecule comprising a large highly immunogenic extracellular mucin-like domain with a large number of twenty amino acid tandem repeats, a transmembrane region, and a cytoplasmic tail (Quin et al., 2000; McGucken et al., 1995; Dong et al., 1997).

In most epithelial adenocarcinomas including breast and pancreas, MUC1 is overexpressed and aberrantly glycosylated. Adenocarcinoma of the breast and pancreas not only overexpress MUC1 but also shed MUC1 into the circulation. High MUC1 serum levels are associated with progressive disease. MUC1 has been exploited as a prospective biomarker because of the complex and heterogeneous nature of the epitopes expressed within the antigen. MUC1 synthesized by cancerous tissues (e.g., tumor associated MUC1) usually displays an aberrant oligosaccharide profile, which gives rise to the expression of neomarkers such as sialyl-Lea (assayed in the CA19-9 test), sialyl-Lex, and sialyl-Tn (TAG-72), as well as the cryptic epitopes such as Tn.

Several antibodies are being developed against MUC1 for therapeutic use. Pemtumomab (also known as HMFG1) is in Phase III clinical trials as a carrier to deliver the radioisotope Yttrium-90 into tumors in ovarian cancer (reviewed in Scott et al., 2012). CA15-3 (also the HMFG1 antibody), CA27-29, and CA19-9 are all antibodies to MUC1 that are used to assess levels of circulating MUC1 in patients with cancer. However, these antibodies have shown limited utility as therapeutic agents or as biomarkers because they cannot distinguish effectively between MUC1 expressed on normal versus transformed tumor epithelia. In other words, none of these antibodies appear to be targeted to a tumor-specific MUC1 epitope.

A new antibody that is highly specific for a tumor-specific form of MUC1 (tMUC) is designated TAB-004 and is described in U.S. Pat. No. 8,518,405 (see also Curry et al., 2013). While Pemtumomab (HMFG1) was developed using human milk fat globules as the antigen (Parham et al., 1988), TAB-004 was developed using tumors expressing an altered form of MUC1 (Tinder et al., 2008). TAB-004 recognizes the altered glycosylated epitope within the MUC1 tandem repeat sequence. This area is accessible for antigenic detection in tMUC but is blocked from antigenic detection in normal MUC1 by large branches of glycosylation (Gendler, 2001; Mukherjee et al., 2003b; Hollingsworth & Swanson, 2004; Kufe, 2009). Importantly, TAB-004 is different from the epitopes recognized by other MUC1 antibody and has unique complementary determinant regions (CDRs) of the heavy and light chains. The antibody binds the target antigen with a high binding affinity at 3 ng/ml (20 pM) and does not bind unrelated antigens (Curry et al., 2013). Thus, TAB-004 distinguishes between normal and tumor form of MUC1 while HMFG1 (Pemtumomab) does not (see U.S. Pat. No. 8,518,405).

In embodiments, the first antigen binding molecule is a chimeric antigen receptor (CAR) and the second antigen binding molecule is a T-cell Receptor (TCR). In embodiments, the TCR is modified TCR. In embodiments, the TCR is derived from spontaneously occurring tumor-specific T-cells in patients. In embodiments, the TCR binds a tumor antigen. In embodiments, the tumor antigen comprises CEA, gp100, MART-1, p53, MAGE-A3, or NY-ESO-1, or the TCR comprises TCRγ and TCRδ Chains or TCRα and TCRβ chains, or a combination thereof.

In embodiments, the CAR comprises an antigen binding domain, a transmembrane domain, and a co-stimulatory domain. In embodiments, the co-stimulatory domain comprises the intracellular domain of a co-stimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83, or a combination thereof. In embodiments, the CAR comprises an antigen binding domain, a transmembrane domain, a co-stimulatory domain, and a CD3 zeta domain. In embodiments, the first and second antigen binding domain is a Fab or a scFv.

In embodiments, the modified cell is a T-cell, NK cell, or dendritic cells. In embodiments, the modified cell is a T-cell.

In embodiments, immune checkpoint molecule is modified PD-1. In embodiments, the modified PD-1 lacks a functional PD-1 intracellular domain for PD-1 signal transduction, interferes with a pathway between PD-1 of a human T-cell of the human cells and PD-L1 of a certain cell, comprises a PD-1 extracellular domain or a PD-1 transmembrane domain, or a combination thereof, comprises a modified PD-1 intracellular domain comprising a substitution or deletion as compared to a wild-type PD-1 intracellular domain, or comprises a soluble receptor comprising a PD-1 extracellular domain that binds to PD-L1 of a certain cell. In embodiments, an inhibitory effect of PD-L1 on cytokine production of the T-cells of the population is less than an inhibitory effect of PD-L1 on cytokine production of T-cells that do not comprise at least a part of the nucleic acid sequence that encodes the modified PD-1. In embodiments, the T-cells are human T-cells.

In embodiments, the modified cell is engineered to express and secrete a therapeutic agent such as a cytokine. In embodiments, the therapeutic agent comprises IFN-γ. In embodiments, the therapeutic agent comprises IL-6 or IFN-γ, or a combination thereof. In embodiments, the therapeutic agent comprises IL-15 or IL-12, or a combination thereof. In embodiments, a molecule, or the therapeutic agent comprises a recombinant or native cytokine. In embodiments, the therapeutic agent comprises an FC fusion protein associated with a small protein. In embodiments, the molecule comprises IL-12, IL-15, IL-6 or IFN-γ.

In embodiments, expression of the therapeutic agent and/or the modified PD-1 is regulated by Hif1a, NFAT, FOXP3, and/or NFkB. In embodiments, the modified cell includes a nucleic acid comprising the isolated nucleic acids described herein, wherein the isolated nucleic acid includes a promoter comprising a binding site for a transcription modulator (e.g., transcription factors) that modulates the expression of the therapeutic agent in the cell. These constructs can be placed into vectors (e.g., lentiviral vectors) either in a forward or reverse direction. In embodiments, the transcription modulator includes Hif1a, NFAT, FOXP3, and/or NFkB. In embodiments, the promoter is responsive to the transcription modulator. In embodiments, the promoter is operably linked to the nucleic acid sequence encoding the therapeutic agent, such that the promoter drives expression of the therapeutic agent in the cell. In embodiments, the therapeutic agent is ligated to a specific promoter such as to induce expression of the therapeutic agent in a desired condition. The promoter is divided into two parts, a specific regulatory region containing a transcription factor binding site, plus a minimal promoter. More information about NFAT may be found at WO2018006882, which is incorporated herein by reference. In embodiments, the transcription modulator includes STAT5 response element, (activated by cytokines such as IL2, IL3, IL7, IL15, and the transcription factor associated with it is STAT5), STAT3 response element, (activated by cytokines such as IL6, transcription factor is STAT3), Interferon Stimulated Response Element, (activated by IFN-α, transcription factors are STAT1 and STAT2), AP1 Response Element, (activated by MAPK/JNK pathway, transcription factor is AP1), SMAD Binding Element (activated by TGF-β, transcription factors are SMAD3 and SMAD4), Serum Response Element (activated by MAP/ERK pathway, transcription factor is Elk1/SRF), Serum Response Factor Response Element (activated by the RhoA pathway, the transcription factor is SRF), Cyclic AMP response element (activated by cAMP/PKA pathway, transcription factor is CREB), or TCF-LEF Response Element (activated by Wnt pathway, transcription factor is TCF-LEF).

In embodiments, the therapeutic agent includes an antibody reagent (e.g., a single chain antibody (e.g., scFv), a single domain antibody (e.g., a camelid antibody), or a bispecific antibody reagent (e.g., a bispecific T-cell engager (BiTE)). In embodiments, the therapeutic agent includes a cytokine or a cytokine related molecule. Examples of the cytokines and the related molecules include IL-1P, IL-2, IL-4, IL-5, IL-6, IL-8, IL-10, IL-12, IL-13, IL-15, IL-17, IL-1Ra, IL-2R, IFN-γ, IFN-γ, MIP-In, MIP-IP, MCP-1, TNFα, GM-CSF, GCSF, CXCL9, CXCL10, CXCR factors, VEGF, RANTES, EOTAXIN, EGF, HGF, FGF-P, CD40, CD40L, and any combination thereof. In embodiments, the cytokines include proinflammatory cytokines such as: IFN-γ, IL-15, IL-4, IL-10, TNFα, IL-8, IL-5, IL-6, GM-CSF, ferritin, and MIP-1α. In embodiments, the therapeutic agent comprises IL17, CCL19, or a combination thereof. In these instances, IL-17 and CCL19 secreted by fibroblastic reticular cells in lymphoid organs may effectively recruit peripheral T-cells and DC cells to maintain T-cell zone. For example, IFN-γ has been approved by FDA to treat patients with malignant osteoporosis (e.g., Journal of Pediatrics 121(1):119-24 Aug. 1992).

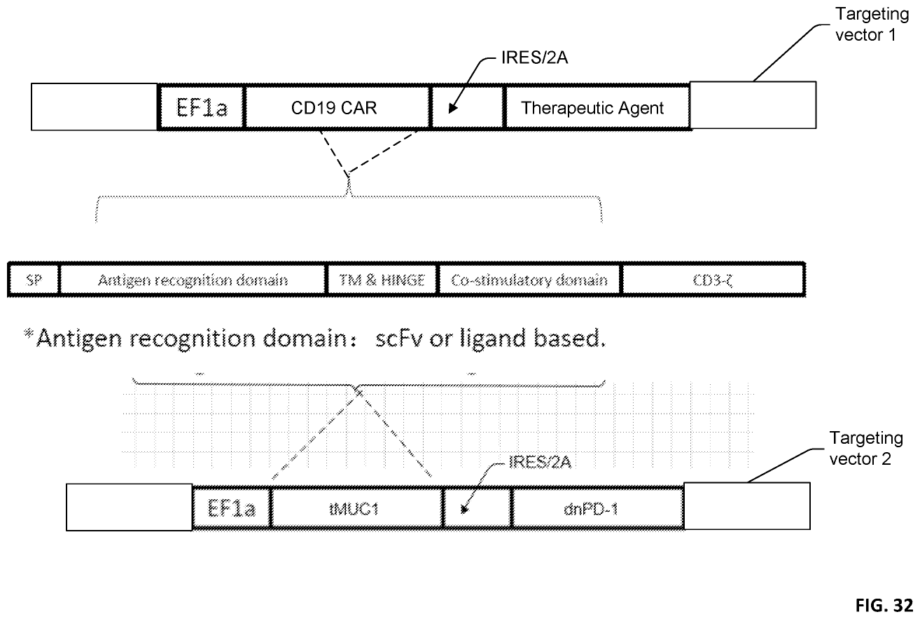

In embodiments, the modified cell comprises a first targeting vector and a second targeting vector, the first targeting vector comprising a nucleic acid sequence encoding a CAR binding a blood antigen and the therapeutic agent, and the second targeting vector comprises a nucleic acid sequence encoding a CAR biding solid tumor antigen and a dominant negative form of the immune checkpoint molecule. In embodiments, the modified cell comprises a first targeting vector and a second targeting vector, the first targeting vector comprising a nucleic acid sequence encoding a CAR binding CD19 and the therapeutic agent, and the second targeting vector comprises a nucleic acid sequence encoding a CAR biding tMUC1 and a dominant negative form of PD-1. An example of the target vectors is provided in FIG. 32. In embodiments, dnPD-1 may enhance persistence of tMUC1 CAR to infiltrate tumor, and the therapeutic agent (e.g., IL-6, IL-12, or IFN-γ) may trigger, cause, and/or enhance immune responses of the subject, together leading enhanced tMUC1 CAR T treatment on solid tumor.

In embodiments, the modified cell is derived from a healthy donor or the subject having the cancer. In embodiments, the modified cell has a reduced expression of endogenous TRAC gene. In embodiments, the modified cell further comprises the endogenous TRAC gene that has been inactivated in the CAR T-cell to avoid GVHD and rejection. For example, the CAR T-cell has a reduced expression of endogenous TRAC gene. In embodiments, the CAR T-cell lacks expression of a functional endogenous TCR and/or produces substantially impaired endogenous TCR on its surface such that the endogenous TCR will not substantially elicit an adverse immune reaction in a host, e.g., a GVHD reaction. In embodiments, progeny of the CAR T-cell may also be reasonably expected to lack expression of a functional endogenous TCR and/or produce substantially impaired endogenous TCR on their surface such that the progeny of the endogenous TCR will not substantially elicit an adverse immune reaction in a host, e.g., a GVHD reaction. In embodiments, the TCR is derived from a healthy human donor having HLA type that matches the recipient. Typically, matching is performed on the basis of variability at three or more loci of the HLA gene, and a perfect match at these loci is preferred. In embodiments, allogeneic transplant donors can be related (usually a closely HLA matched sibling), syngeneic (a monozygotic ‘identical’ twin of the patient) or unrelated (donor who is not related and found to have very close degree of HLA matching). The HLA genes fall in two categories (Type I and Type II). Mismatches of the Type-I genes (i.e. HLA-A, HLA-B, or HLA-C) increase the risk of graft rejection. A mismatch of an HLA Type II gene (i.e. HLA-DR, or HLA-DQB1) increases the risk of graft-versus-host disease. In embodiments, endogenous HLA I gene of the CAR T-cell can be further inactivated to avoid recipient's rejection of the donor's CAR T-cell.

In embodiments, the CAR molecules described herein comprise one or more complementarity-determining regions (CDRs) for binding an antigen of interest. CDRs are part of the variable domains in immunoglobulins and T cell receptors for binding a specific antigen. There are three CDRs for each variable domain. Since there is a variable heavy domain and a variable light domain, there are six CDRs for binding an antigen. Further since an antibody has two heavy chains and two light chains, an antibody has twelve CDRs altogether for binding antigens. In embodiments, the CAR molecules comprise one or more CDRs for binding tMUC 1, PRLR, CLCA1, MUC12, GUCY2C, GPR35, CR1L, MUC 17, TMPRSS11B, MUC21, TMPRSS11E, CD207, SLC30A8, CFC1, SLC12A3, SSTR1, GPR27, FZD10, TSHR, SIGLEC15, SLC6A3, KISS1R, QRFPR, GPR119, CLDN6, UPK2, ADAM12, SLC45A3, ACPP, MUC21, MUC16, MS4A12, ALPP, CEA, EphA2, FAP, GPC3, IL13-Ra2, Mesothelin, PSMA, ROR1, VEGFR-II, GD2, FR-α, ErbB2, EpCAM, EGFRvIII, EGFR, CD19, CD20, CD22, or BCMA.

In embodiments, the population of cells described herein is used in autologous CAR T cell therapy. In embodiments, the CAR T cell therapy is allogenic CAR T cell therapy, TCR T cell therapy, and NK cell therapy.

Embodiments relate to compositions comprising a population of cells including T cells comprising the CARs described herein. Embodiments relate to CARs encoded by the isolated nucleic acid sequences described herein.

The cells, including CAR cells and modified cells, described herein can be derived from stem cells. The stem cells can be adult stem cells, embryonic stem cells, or non-human stem cells, cord blood stem cells, progenitor cells, bone marrow stem cells, induced pluripotent stem cells, totipotent stem cells, or hematopoietic stem cells. The cells can also be a dendritic cell, a NK-cell, a B-cell, or a T-cell selected from the group consisting of inflammatory T-lymphocytes, cytotoxic T-lymphocytes, regulatory T-lymphocytes, and helper T-lymphocytes. In embodiments, the cells can be derived from the group consisting of CD4+T-lymphocytes and CD8+T-lymphocytes. Prior to expansion and genetic modification of the cells described herein, a source of cells can be obtained from a subject through a variety of non-limiting methods. T-cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In embodiments, any number of T-cell lines available and known to those skilled in the art, can be used. In embodiments, the cells can be derived from a healthy donor, from a patient diagnosed with cancer. In embodiments, the cells are part of a mixed population of cells which present different phenotypic characteristics.

The term “stem cell” refers to any type of cell which has the capacity for self-renewal and the ability to differentiate into other kind(s) of cell. For example, a stem cell gives rise either to two daughter stem cells (as occurs in vitro with embryonic stem cells in culture) or to one stem cell and a cell that undergoes differentiation (as occurs e.g. in hematopoietic stem cells, which give rise to blood cells). Different categories of stem cells can be distinguished on the basis of their origin and/or on the extent of their capacity for differentiation into other types of cells Stem cells can include embryonic stem (ES) cells (i.e., pluripotent stem cells), somatic stem cells, induced pluripotent stem cells, and any other types stem cells.

Pluripotent embryonic stem cells can be found in the inner cell mass of a blastocyst and have high innate capacity for differentiation. For example, pluripotent embryonic stem cells have the potential to form any type of cell in the body. When grown in vitro for long periods of time, ES cells maintain pluripotency, and progeny cells retain the potential for multilineage differentiation.

Somatic stem cells can include fetal stem cells (from the fetus) and adult stem cells (found in various tissues, such as bone marrow). These cells have been regarded as having a capacity for differentiation lower than that of the pluripotent ES cells—with the capacity of fetal stem cells being greater than that of adult stem cells; they apparently differentiate into only a limited number of different types of cells and have been described as multipotent. Tissue-specific stem cells normally give rise to only one type of cell. For example, embryonic stem cells can differentiate into blood stem cells (e.g., Hematopoietic stem cells (HSCs)), which can further differentiate into various blood cells (e.g., red blood cells, platelets, white blood cells, etc.).

Induced pluripotent stem cells (iPS cells or iPSCs) can include a type of pluripotent stem cell artificially derived from a non-pluripotent cell (e.g., an adult somatic cell) by inducing expression of specific genes. Induced pluripotent stem cells are similar to naturally occurring pluripotent stem cells, such as embryonic stem (ES) cells, in many aspects, such as the expression of certain stem cell genes and proteins, chromatin methylation patterns, doubling time, embryoid body formation, teratoma formation, viable chimera formation, and potency and differentiability. Induced pluripotent cells can be isolated from adult stomach, liver, skin cells, and blood cells.

In embodiments, the CAR cells, the modified cell, or the cell is a T cell, a NK cell, a macrophage or a dendritic cell. For example, the CAR cells, the modified cell, or the cell is a T cell.

The present disclosure is further described by reference to the following exemplary embodiments and examples. These exemplary embodiments and examples are provided for purposes of illustration only and are not intended to be limiting unless otherwise specified. Thus, the present disclosure should in no way be construed as being limited to the following exemplary embodiments and examples, but rather, should be construed to encompass any and all variations which become evident as a result of the teaching provided herein.

### EXEMPLARY EMBODIMENTS

The following are exemplary embodiments:

1. A method for increasing a number and/or ratio of a subpopulation of immune cells in a subject having cancer and/or increasing or extending persistence of a subpopulation of immune cells infused into a subject having cancer, the method comprising: administering an effective amount of a composition comprising T-cells to the subject, the T-cells including a chimeric antigen receptor (CAR) and a modified PD-1, the modified PD-1 lacking a functional PD-1 intracellular domain; and monitoring T-cell response in the subject, the subpopulation of T-cells comprising naive T-cells, stem cell memory T-cells, and/or central memory T-cells, the number and/or ratio of the subpopulation of immune cells in the subject is increased as compared to the number and/or ratio of the subpopulation of immune cells in the subject administered with corresponding T-cells that do not include the modified PD-1 or the persistence of the subpopulation of the immune cells in the subject is increased or extended as compared to the the persistence of the subpopulation of immune cells in the subject administered with corresponding T-cells that do not include the modified PD-1.
2. The method of embodiment 1, wherein monitoring the T-cell response in the subject comprises at least one of: detecting or measuring mRNA of the modified PD-1; detecting or measuring a number of one or more white blood cells; detecting or measuring a number of at least one of naive T-cells, stem cell memory T-cells, and central memory T-cells; detecting or measuring a CAR copy number; detecting or measuring CD3 positive cell number; detecting or measuring a number of T-cells expressing CAR; and detecting a level of one or more cytokines.
3. The method of embodiment 1 or 2, wherein the T-cells in the subject comprises more memory T-cells than T-cells in a subject administered with T-cells comprising the CAR that does not include the modified PD-1.
4. The method of any one of embodiments 1-3, wherein (i) the modified PD-1 comprises an amino acid sequence of SEQ ID: 36-41 or 55-57; or (ii) the modified PD-1 does not include SEQ ID: 42.5. The method of any one of embodiments 1-4, wherein the T-cells comprise a dominant negative variant of PD-1, and endogenous gene of PD-1 of the T-cells is not disrupted.
6. The method of any one of embodiments 1-5, wherein the CAR comprises an extracellular domain, a transmembrane domain, and an intracellular domain, wherein the extracellular domain binds an antigen.
7. The method of any one of embodiments 1-6, wherein the extracellular domain comprises one or more of amino acid sequences of SEQ ID: 5-17 or 30-35.
8. The method of any one of embodiments 1-7, wherein the T-cells comprise one or more nucleic acid sequences of SEQ ID: 18-29.
9. The method of any one of embodiments 1-8, wherein the intracellular domain comprises a co-stimulatory signaling region that comprises an intracellular domain of a co-stimulatory molecule selected from the group consisting of CD27, CD28, 4-1 BB, OX40, CD30, CD40, PD-1, ICOS, lymphocyte function-associated antigen-1 (LFA-1), CD2, CD7, LIGHT, NKG2C, B7-H3, and any combination thereof.
10. The method of any one of embodiments 1-9, wherein the antigen is Epidermal growth factor receptor (EGFR), Variant III of the epidermal growth factor receptor (EGFRvIII), Human epidermal growth factor receptor 2 (HER2), Mesothelin (MSLN), Prostate-specific membrane antigen (PSMA), Carcinoembryonic antigen (CEA), Disialoganglioside 2 (GD2), Interleukin-13Ra2 (IL13Rα2), Glypican-3 (GPC3), Carbonic anhydrase IX (CAIX), L1 cell adhesion molecule (L1-CAM), Cancer antigen 125 (CA125), Cluster of differentiation 133 (CD133), Fibroblast activation protein (FAP), Cancer/testis antigen 1B (CTAG1B), Mucin 1 (MUC1), Folate receptor-α (FR-α), CD19, FZD10, TSHR, PRLR, Muc 17, GUCY2C, CD207, CD3, CD5, B-Cell Maturation Antigen (BCMA), or CD4.
11. The method of any one of embodiments 1-10, wherein the composition comprises T-cells comprising a CAR binding to a solid tumor antigen and a CAR binding a white cell antigen.
12. The method of any one of embodiments 1-11, wherein the white cell antigen is a B cell antigen.
13. The method of any one of embodiments 1-12, wherein the solid tumor antigen is tMUC 1, PRLR, CLCA1, MUC12, GUCY2C, GPR35, CR1L, MUC 17, TMPRSS11B, MUC21, TMPRSS11E, CD207, SLC30A8, CFC1, SLC12A3, SSTR1, GPR27, FZD10, TSHR, SIGLEC15, SLC6A3, KISS1R, QRFPR, GPR119, CLDN6, UPK2, ADAM12, SLC45A3, ACPP, MUC21, MUC16, MS4A12, ALPP, CEA, EphA2, FAP, GPC3, IL13-Rα2, Mesothelin, PSMA, ROR1, VEGFR-II, GD2, FR-α, ErbB2, EpCAM, EGFRvIII, or EGFR.
14. The method of any one of embodiments 1-13, wherein the B cell antigen is CD19, CD20, CD22, or BCMA.
15. The method of any one of embodiments 1-14, wherein expression of the modified PD-1 is regulated by Hif1a, NFAT, FOXP3, and/or NFkB.
16. The method of any one of embodiments 1-15, wherein the T-cells express and/or secrete a therapeutic agent comprising at least one of IFNγ, IL-15, or IL-12.17. A pharmaceutical composition comprising human T-cells, wherein the human T-cells comprising: a first nucleic acid sequence that encodes truncated CTLA4 that reduces an inhibitory effect of CTLA4 ligand 1 on the human T-cells, the truncated CTLA4 lacking a functional CTLA4 intracellular domain; and a second nucleic acid sequence that encodes a chimeric antigen receptor (CAR) comprising an extracellular domain that binds a tumor antigen of a target cell, a transmembrane domain, and an intracellular domain comprising a CD3-zeta signaling domain and a signaling domain of a co-stimulatory molecule, wherein the truncated CTLA4 and the CAR are expressed as gene products that are separate polypeptides.
18. The pharmaceutical composition of embodiment 17, wherein the truncated CTLA4 interferes with a pathway between CTLA4 of a human T-cell and CTLA4 ligand of a target cell.
19. The pharmaceutical composition of embodiment 17 or 18, wherein the truncated CTLA4 comprises a CTLA4 extracellular domain or a CTLA4 transmembrane domain, or a combination thereof.
20. The pharmaceutical composition of any one of embodiments 17-19, wherein the truncated CTLA4 comprises an amino acid sequences of SEQ ID: 44-52.
21. A population of CAR cells comprising the first and second nucleic acid sequences of any of embodiments 17-20.
22. A pharmaceutical composition comprising the population of the CAR cells of embodiment 21.
23. A method of inducing or causing T-cell response in a subject in need thereof and/or treating a tumor of the subject, the method comprising administering an effective amount of the composition of embodiment 22 to the subject.
24. A method for increasing a number and/or ratio of an subpopulation of immune cells and/or increasing or extending persistence of the subpopulation of immune cells, the method comprising: introducing a population of T-cells with a nucleic acid sequence encoding a chimeric antigen receptor (CAR); and introducing with the T-cells a nucleic acid sequence encoding a modified PD-1, the modified PD-1 lacking a functional PD-1 intracellular domain, the subpopulation of immune cells comprising naive T-cells, stem cell memory T-cells, and/or central memory T-cells, the number and/or ratio of the subpopulation of immune cells is increased in the population of T-cells as compared to the corresponding population of T-cells that do not include the modified PD-1 or the persistence of the subpopulation of immune cells is increased or extended as compared to the corresponding population of T-cells that do not include the modified PD-1.
25. A method of causing or enhancing T-cell response in a subject in need thereof, enhancing T-cell expansion in a subject, and/or treating a tumor of the subject, the method comprising: introducing a plurality of nucleic acid sequences encoding a first antigen binding molecule and a second antigen binding molecule into a population of cells; and administering an effective amount of the composition of the population of cells into the subject.
26. A method of causing or enhancing T-cell response in a subject in need thereof, enhancing T-cell expansion in a subject, and/or treating a tumor of the subject, the method comprising: obtaining a population of cells comprising a first antigen binding molecule; obtaining a population of cells comprising a second antigen binding molecule; and administering an effective amount of the composition of the population of cells comprising the first antigen binding molecule and the population of cells comprising the second antigen binding molecule, respectively or at the same time.
27. A method of causing or enhancing cell response in a subject in need thereof, enhancing T-cell expansion in a subject, and/or treating a tumor of the subject, the method comprising: obtaining a population of cells comprising a first antigen binding molecule; obtaining a population of cells comprising a second antigen binding molecule; obtaining a population of cells comprising the first antigen binding molecule and the second binding molecule in the same cell; and administering an effective amount of the population of cells comprising the first antigen binding molecule, the population of cells comprising the second antigen binding molecule, and the population of cells comprising the first antigen binding molecule and the second binding molecule, sequentially or at the same time.
28. A mixture of modified cells, wherein the modified cells comprise a plurality of nucleic acid sequences encoding a first antigen binding molecule and a second antigen binding molecule.
29. A mixture of modified cells, wherein the modified cells comprise at least two of the following: a population of cells comprising a first antigen binding molecule, a population of cells comprising a second antigen binding molecule, and a population of cells comprising the first antigen binding molecule and the second binding molecule in the same cell.
30. The mixture of modified cells of embodiments 28 or 29, wherein the mixture of cells is capable of causing or enhancing cell response in a subject in need thereof, enhancing T-cell expansion in a subject, and/or treating a tumor of the subject.
31. The mixture or method of one of embodiments 25-30, wherein the population of cells comprising the first antigen binding molecule comprise a nucleic acid sequence encoding a therapeutic agent.
32. The mixture or method of one of embodiments 25-31, wherein the population of cells comprising the second antigen binding molecule comprises a nucleic acid sequence encoding a dominant negative form of an immune checkpoint molecule.
33. The mixture or the method of one of embodiments 25-31, wherein the first binding molecule is a chimeric antigen receptor (CAR) binding CD19, the second binding molecule is a CAR binding tMUC1.
34. The mixture or the method of embodiment 32, wherein the dominant negative form of the immune checkpoint molecule is modified PD-1 lacking a functional PD-1 intracellular domain for PD-1/PD-L1 signal transduction.
35. The mixture or the method of any one of embodiments 25-32, wherein the first antigen binding molecule is a first CAR.
36. The mixture or the method of any one of embodiments 25-32, wherein the first antigen binding molecule binds a cell surface molecule of a blood cell or a white blood cell, the first antigen binding molecule binds an antigen of a white blood cell (WBC), or the first antigen binding molecule binds a molecule associated with blood of the subject.
37. The mixture or the method of embodiment 36, wherein the antigen of white blood cell comprises CD19, CD22, CD20, BCMA, CD5, CD7, CD2, CD16, CD56, CD30, CD14, CD68, CD11b, CD18, CD169, CD1c, CD33, CD38, CD138, CD13, B7, CAIX, CD123, CD133, CD171, CD171/L1-CAM, CEA, Claudin 18.2, cMet, CS1, CSPG4, Dectin1, EGFR, EGFR vIII, EphA2, ERBB receptors, ErbB T4, ERBB2, FAP, Folate receptor 1, FITC, Folate receptor 1, FSH, GD2, GPC3, HA-1 H/HLA-A2, HER2, IL-11Ra, IL13 receptor a2, IL13R, IL13Rα2 (zetakine), Kappa, Leukemia, LewisY, Mesothelin, MUC1, NKG2D, NY-ESO-1, PSMA, ROR-1, TRAIL-receptor1, or VEGFR2.
38. The mixture or the method of embodiment 36, wherein the WBC is a granulocyte, a monocyte and or lymphocyte.
39. The mixture or the method of embodiment 36, wherein the WBC is a B cell.
40. The mixture or the method of embodiment 36, wherein the cell surface molecule of the WBC is CD19.
41. The mixture or the method of any one of embodiments 25-32, wherein the first binding molecule binds to CD19, CD22, CD20, BCMA, CD5, CD7, CD2, CD16, CD56, CD30, CD14, CD68, CD11b, CD18, CD169, CD1c, CD33, CD38, CD138, orCD13, or the cell surface molecule of the WBC is CD19, CD20, CD22, or BCMA.
42. The mixture or the method of any one of embodiments 25-41, wherein the second binding molecule and the first binding molecule bind to different antigens.
43. The mixture or the method of one of embodiments 25-41, wherein the second binding molecule binds a solid tumor antigen.
44. The mixture or the method of embodiment 43, wherein the solid tumor antigen comprises MUC 1, PRLR, CLCA1, MUC12, GUCY2C, GPR35, CR1L, MUC 17, TMPRSS11B, MUC21, TMPRSS11E, CD207, SLC30A8, CFC1, SLC12A3, SSTR1, GPR27, FZD10, TSHR, SIGLEC15, SLC6A3, KISS1R, QRFPR, GPR119, CLDN6, UPK2, ADAM12, SLC45A3, ACPP, MUC21, MUC16, MS4A12, ALPP, CEA, EphA2, FAP, GPC3, IL13-Ra2, Mesothelin, PSMA, ROR1, VEGFR-II, GD2, FR-α, ErbB2, EpCAM, EGFRvIII, or EGFR.
45. The mixture or the method of embodiment 43, wherein the solid tumor antigen comprisesB7, CAIX, CD123, CD133, CD171, CD171/L1-CAM, CEA, Claudin 18.2, cMet, CS1, CSPG4, Dectin1, EGFR, EGFR vIII, EphA2, ERBB receptors, ErbB T4, ERBB2, FAP, Folate receptor 1, FITC, Folate receptor 1, FSH, GD2, GPC3, HA-1 H/HLA-A2, HER2, IL-11Ra, IL13 receptor a2, IL13R, IL13Rα2 (zetakine), Kappa, Leukemia, LewisY, Mesothelin, MUC1, NKG2D, NY-ESO-1, PSMA, ROR-1, TRAIL-receptor1, or VEGFR2.
46. The mixture or the method of embodiment 44, wherein the MUC1 is a tumor form of human MUC1 (tMUC1).
47. The mixture or the method of embodiment 44, wherein the MUC1 is a tumor-exclusive epitope of a human MUC1, and the first CAR and the second CAR are expressed as polypeptides.
48. The mixture or the method of any one of embodiments 25-41, wherein the first antigen binding molecule is a chimeric antigen receptor (CAR) and the second antigen binding molecule is a T-cell Receptor (TCR).
49. The mixture or the method of embodiment 48, wherein the TCR is modified TCR.
50. The mixture or the method of embodiment 48, wherein the TCR is derived from spontaneously occurring tumor-specific T-cells in patients.
51. The mixture or the method of embodiment 48, wherein the TCR binds a tumor antigen.
52. The mixture or the method of embodiment 48, wherein the tumor antigen comprises CEA, gp100, MART-1, p53, MAGE-A3, or NY-ESO-1, or the TCR comprises TCRγ and TCRδ Chains or TCRα and TCRP chains, or a combination thereof.
53. The mixture or the method of any one of embodiments 25-52, wherein the CAR comprises an antigen binding domain, a transmembrane domain, and a co-stimulatory domain.
54. The mixture or the method of embodiment 53, wherein the co-stimulatory domain comprises the intracellular domain of a co-stimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83, or a combination thereof.
55. The mixture or the method of any one of embodiments 25-52, wherein the CAR comprises an antigen binding domain, a transmembrane domain, a co-stimulatory domain, and a CD3 zeta domain.
56. The mixture or the method of embodiments 25-53, wherein the first and second antigen binding domain is a Fab or a scFv.
57. The mixture or the method of embodiments 25-56, wherein the mixture comprises T-cells, NK cells, macrophages, dendritic cells, or a combination thereof.
58. The mixture or the method of embodiments 25-57, wherein the mixture comprises T-cells.
59. The mixture or the method of any one of embodiments 25-58, wherein the immune checkpoint molecule is selected from the group consisting of programmed death 1 (PD-1), cytotoxic T lymphocyte antigen-4 (CTLA-4), B- and T-lymphocyte attenuator (BTLA), T-cell immunoglobulin mucin-3 (TIM-3), lymphocyte-activation protein 3 (LAG-3), T-cell immunoreceptor with Ig and ITIM domains (TIGIT), leukocyte-associated immunoglobulin-like receptor 1 (LAIRI), natural killer cell receptor 2B4 (2B4), and CD 160.
60. The mixture or the method of embodiment 59, wherein immune checkpoint molecule is modified PD-1.
61. The mixture or the method of embodiment 59, wherein (i) the modified PD-1 lacks a functional PD-1 intracellular domain for PD-1 signal transduction, interferes with a pathway between PD-1 of a human T-cell and PD-L1 of a certain cell, comprises a PD-1 extracellular domain or a PD-1 transmembrane domain, or a combination thereof; or (ii) a modified PD-1 intracellular domain comprises a substitution or deletion as compared to a wild-type PD-1 intracellular domain, or comprises a soluble receptor comprising a PD-1 extracellular domain that binds to PD-L1 of a certain cell.
62. The mixture or the method of embodiment 59, wherein an inhibitory effect of PD-L1 on cytokine production of the T-cells of the population is less than an inhibitory effect of PD-L1 on cytokine production of T-cells that do not comprise at least a part of the nucleic acid sequence that encodes the modified PD-1. In embodiments, the T cells are human T cells.
63. The mixture or the method of any one of embodiments 25-62, wherein the mixture is engineered to express and secrete a therapeutic agent such as a cytokine.
64. The mixture or the method of embodiment 63, wherein the therapeutic agent comprises IFN-γ.
65. The mixture or the method of embodiment 42, wherein the therapeutic agent comprises IL-6, IFN-γ, or a combination thereof.
66. The mixture or the method of embodiment 63, wherein the therapeutic agent comprises IL-15, IL-12, or a combination thereof.
67. The mixture or the method of embodiment 63, wherein the molecule or the therapeutic agent comprises a recombinant or native cytokine.
68. The mixture or the method of embodiment 63, wherein the therapeutic agent comprises a FC fusion protein associated with a molecule.
69. The mixture or the method of embodiment 63, wherein the molecule comprises IL-12, IL-15, IL-6 or IFN-γ.
70. The mixture or the method of embodiment 63, wherein the therapeutic agent is regulated by Hif1a, NFAT, FOXP3, and/or NFkB.
71. The mixture or the method of any one of embodiments 25-70, wherein the cells comprises a first targeting vector and a second targeting vector, the first targeting vector comprising a nucleic acid sequence encoding a CAR binding a blood antigen and the therapeutic agent, and the second targeting vector comprises a nucleic acid sequence encoding a CAR biding solid tumor antigen and a dominant negative form of the immune checkpoint molecule.
72. The mixture or the method of any one of embodiments 25-70, wherein the cells comprise a first targeting vector and a second targeting vector, the first targeting vector comprising a nucleic acid sequence encoding a CAR binding CD19 and a therapeutic agent, and the second targeting vector comprises a nucleic acid sequence encoding a CAR biding tMUC1 and a dominant negative form of PD-1.
73. The mixture or the method of any one of embodiments 25-71, wherein the cells are derived form a healthy donor or the subject having the cancer.
74. The mixture or the method of any one of embodiments 25-72, wherein the modified cells have a reduced expression of endogenous TRAC gene.
75. A composition comprising a first population of cells comprising a CAR binding an antigen and a second population of cells comprising a CAR and/or a dominant negative form of immune checkpoint molecule.
76. The composition of embodiment 75, wherein the first population of cells do not comprise the dominant negative form of immune checkpoint molecule.
77. A method of enhancing expansion of the population of the second cells, the method comprising administering an effective amount of the composition of any one of embodiments 75-76 to a subject having cancer associated with a tumor antigen that the CAR binds.
78. A method of enhancing T-cell response in a subject or treating the subject having cancer, the method comprising administering an effective amount of the composition of any one of embodiments 75-76 to the subject having cancer associated with a tumor antigen that the CAR binds.
79. A method of enhancing expansion of cells in a subject, the method comprising: contacting cells with a first vector comprising a first nucleic acid sequence encoding the CAR and a second vector comprising a second nucleic acid sequence encoding the CAR and/or dominant negative form of immune checkpoint molecule to obtain the composition of any one of embodiments 75-76; and administering an effective amount of the composition to the subject having cancer associated with a tumor antigen that the CAR binds.
80. A method of enhancing T-cell expansion in a subject, the method comprising: contacting T-cells with a first vector comprising a first nucleic acid sequence encoding the CAR and a second vector comprising a second nucleic acid sequence encoding a dominant negative form of immune checkpoint molecule to obtain a composition; and administering an effective amount of the composition to the subject having cancer associated with a tumor antigen that the CAR binds.
81. A method of enhancing T-cell expansion in a subject, the method comprising: administering an effective amount of T-cells comprising a CAR and a dominant negative form of immune checkpoint molecule to the subject having cancer associated with a tumor antigen that the CAR binds.
82. The method of embodiment 80 and 81, wherein the enhanced T-cell expansion is measured based on numbers of T-cells having the CAR as compared to those of T-cell expansion in response to T-cell having the CAR but not having the dominant negative form of immune checkpoint molecule.
83. The method of embodiment 80 and 81, wherein an increase of T-cell numbers of a subject infused with CAR T-cells having the CAR and the dominant negative form of immune checkpoint molecule is higher than that of a subject infused with CAR T-cells having the CAR but not the dominant negative form of immune checkpoint molecule.
84. The composition or the method of any one of embodiments 75-83, wherein the inhibitory immune checkpoint molecule is selected from the group consisting of programmed death 1 (PD-1), cytotoxic T lymphocyte antigen-4 (CTLA-4), B- and T-lymphocyte attenuator (BTLA), T-cell immunoglobulin mucin-3 (TIM-3), lymphocyte-activation protein 3 (LAG-3), T-cell immunoreceptor with Ig and ITIM domains (TIGIT), leukocyte-associated immunoglobulin-like receptor 1 (LAIRI), natural killer cell receptor 2B4 (2B4), and CD 160.
85. The composition or the method of any one of embodiments 75-83, wherein the inhibitory immune checkpoint molecule is PD-1.
86. The method of any one of embodiments 79 and 80, wherein the first vector and the second vector are lentiviral vectors.
87. The composition or the method of any one of embodiments 75-86, wherein the antigen comprises a surface molecule of a white blood cell (WBC), a tumor antigen, or a solid tumor antigen.
88. The composition or the method of any one of embodiments 75-87, wherein the cells are T-cells, NK cells, macrophages, or dendritic cells.
89. The composition or the method of embodiment 88, wherein the WBC is a granulocyte, a monocyte, or lymphocyte.
90. The composition or the method of embodiment 88, wherein the WBC is a B cell.
91. The composition or the method of embodiment 88, wherein the cell surface molecule of the WBC is CD19, CD22, CD20, BCMA, CD5, CD7, CD2, CD16, CD56, CD30, CD14, CD68, CD11b, CD18, CD169, CD1c, CD33, CD38, CD138, or CD13.
92. The composition or the method of embodiment 88, wherein the cell surface molecule of the WBC is CD19, CD20, CD22, or BCMA.
93. The composition or the method of embodiment 88, wherein the cell surface molecule of the WBC is CD19.
94. The composition or the method of embodiment 88, wherein the tumor antigen is a solid tumor antigen.
95. The composition or the method of embodiment 88, wherein the solid tumor antigen is tMUC 1, PRLR, CLCA1, MUC12, GUCY2C, GPR35, CR1L, MUC 17, TMPRSS11B, MUC21, TMPRSS11E, CD207, SLC30A8, CFC1, SLC12A3, SSTR1, GPR27, FZD10, TSHR, SIGLEC15, SLC6A3, KISS1R, QRFPR, GPR119, CLDN6, UPK2, ADAM12, SLC45A3, ACPP, MUC21, MUC16, MS4A12, ALPP, CEA, EphA2, FAP, GPC3, IL13-Ra2, Mesothelin, PSMA, ROR1, VEGFR-II, GD2, FR-α, ErbB2, EpCAM, EGFRvIII, or EGFR.
96. The composition or the method of embodiment 88, wherein the solid tumor antigen comprises tumor associated MUC1.
97. The composition or the method of any one of embodiments 75-96, wherein the CAR comprises the antigen binding domain, a transmembrane domain, a co-stimulatory domain, and a CD3 zeta domain.
98. The composition or the method of embodiment 97, wherein the co-stimulatory domain comprises the intracellular domain of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83, or a combination thereof.
99. A modified cell comprising a plurality of nucleic acid sequences encoding a first antigen binding molecule, a second antigen binding molecule, and a dominant negative form of an immune checkpoint molecule.
100. A modified cell comprising a first binding molecule, a second binding molecule, and a dominant negative form of an immune checkpoint molecule.
101. A method of preparing modified cells, the method comprising: administering an effective amount of modified cells of any one of embodiments 99 and 100.
102. A method of causing or enhancing T-cell response, treating a subject having cancer, enhancing cellular treatment on a subject having cancer, or inhibiting growth of tumor cells, the method comprising: introducing into a cell with a plurality of nucleic acid sequences encoding a first antigen binding molecule, a second antigen binding molecule, and a dominant negative form of an immune checkpoint molecule.
103. The modified cell or the method of any one of embodiments 99-102, wherein the first binding molecule, the second binding molecule, and the dominant negative of the immune checkpoint molecule are expressed as gene products that are separate polypeptides.
104. The modified cell or the method of embodiment 103, wherein the first binding molecule is a chimeric antigen receptor (CAR) binding CD19, the second binding molecule is a CAR binding tMUC1, and the dominant negative form of the immune checkpoint molecule is modified PD-1 lacking a functional PD-1 intracellular domain for PD-1/PD-L1 signal transduction.
105. The modified cell or the method of any one of embodiments 99-102, wherein the modified cell comprises a bispecific chimeric antigen receptor (CAR) comprising the first binding molecule and the second binding molecule, the first binding molecule and the second binding molecule are binding domains of the bispecific CAR, the bispecific CAR and the dominant negative of the immune checkpoint molecule are expressed as gene products that are separate polypeptides.
106. The modified cell or the method of any one of embodiments 99-105, wherein the first antigen binding molecule is a first CAR.
107. The modified cell or the method of any one of embodiments 99-105, wherein the first antigen binding molecule binds a surface molecule of a blood cell or a white blood cell, the first antigen binding molecule binds an antigen of a white blood cell (WBC).
108. The modified cell or the method of any one of embodiments 99-105, wherein the first antigen binding molecule binds a molecule associated with blood of the subject.
109. The modified cell or the method of any one of embodiments 107 and 108, wherein the antigen of white blood cell comprises CD19, CD22, CD20, BCMA, CD5, CD7, CD2, CD16, CD56, CD30, CD14, CD68, CD11b, CD18, CD169, CD1c, CD33, CD38, CD138, CD13, B7, CAIX, CD123, CD133, CD171, CD171/L1-CAM, CEA, Claudin 18.2, cMet, CS1, CSPG4, Dectin1, EGFR, EGFR vIII, EphA2, ERBB receptors, ErbB T4, ERBB2, FAP, Folate receptor 1, FITC, Folate receptor 1, FSH, GD2, GPC3, HA-1 H/HLA-A2, HER2, IL-11Ra, IL13 receptor a2, IL13R, IL13Rα2 (zetakine), Kappa, Leukemia, LewisY, Mesothelin, MUC1, NKG2D, NY-ESO-1, PSMA, ROR-1, TRAIL-receptor1, or VEGFR2.
110. The modified cell or the method of any one of embodiments 99-105, wherein the WBC is a granulocyte, a monocyte, and or lymphocyte.
111. The modified cell or the method of any one of embodiments 99-105, wherein the WBC is a B cell.
112. The modified cell or the method of any one of embodiments 99-105, wherein the cell surface molecule of the WBC is CD19.
113. The modified cell or the method of any one of embodiments 99-105, wherein the first binding molecule binds to CD19, CD22, CD20, BCMA, CD5, CD7, CD2, CD16, CD56, CD30, CD14, CD68, CD11b, CD18, CD169, CD1c, CD33, CD38, CD138, or CD13, or the cell surface molecule of the WBC is CD19, CD20, CD22, or BCMA.
114. The modified cell or the method of any one of embodiments 99-113, wherein the second binding molecule and the first binding molecule bind to different antigens.
115. The modified cell or the method of any one of embodiments 99-113, wherein the second binding molecule binds a solid tumor antigen.
116. The modified cell or the method of embodiment 115, wherein the solid tumor antigen comprises MUC1, PRLR, CLCA1, MUC12, GUCY2C, GPR35, CR1L, MUC 17, TMPRSS11B, MUC21, TMPRSS11E, CD207, SLC30A8, CFC1, SLC12A3, SSTR1, GPR27, FZD10, TSHR, SIGLEC15, SLC6A3, KISS1R, QRFPR, GPR119, CLDN6, UPK2, ADAM12, SLC45A3, ACPP, MUC21, MUC16, MS4A12, ALPP, CEA, EphA2, FAP, GPC3, IL13-Ra2, Mesothelin, PSMA, ROR1, VEGFR-II, GD2, FR-α, ErbB2, EpCAM, EGFRvIII, or EGFR.
117. The modified cell or the method of embodiment 115, wherein the solid tumor antigen comprises B7, CAIX, CD123, CD133, CD171, CD171/L1-CAM, CEA, Claudin 18.2, cMet, CS1, CSPG4, Dectin1, EGFR, EGFR vIII, EphA2, ERBB receptors, ErbB T4, ERBB2, FAP, Folate receptor 1, FITC, Folate receptor 1, FSH, GD2, GPC3, HA-1 H/HLA-A2, HER2, IL-11Ra, IL13 receptor a2, IL13R, IL13Rα2 (zetakine), Kappa, Leukemia, LewisY, Mesothelin, MUC1, NKG2D, NY-ESO-1, PSMA, ROR-1, TRAIL-receptor1, or VEGFR2.
118. The modified cell or the method of embodiment 115, wherein the MUC1 is a tumor form of human MUC1 (tMUC1).
119. The modified cell or the method of embodiment 115, wherein the MUC1 is a tumor-exclusive epitope of a human MUC1, and the first CAR and the second CAR are expressed as polypeptides.
120. The modified cell or the method of any one of embodiments 99-103 and 105-111 wherein the first antigen binding molecule is a chimeric antigen receptor (CAR) and the second antigen binding molecule is a T-cell Receptor (TCR).
121. The modified cell or the method of embodiment 120, wherein the TCR is modified TCR.
122. The modified cell or the method of embodiment 120, wherein the TCR is derived from spontaneously occurring tumor-specific T-cells in patients.
123. The modified cell or the method of embodiment 120, wherein the TCR binds a tumor antigen.
124. The modified cell or the method of embodiment 120, wherein the tumor antigen comprises CEA, gp100, MART-1, p53, MAGE-A3, or NY-ESO-1, or the TCR comprises TCRγ and TCRδ Chains or TCRα and TCRP chains, or a combination thereof.
125. The modified cell or the method of any one of embodiments 99-124, wherein the CAR comprises an antigen binding domain, a transmembrane domain, and a co-stimulatory domain.
126. The modified cell or the method of embodiment 125, wherein the co-stimulatory domain comprises the intracellular domain of a co-stimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83, or a combination thereof.
127. The modified cell or the method of any one of embodiments 99-126, wherein the CAR comprises an antigen binding domain, a transmembrane domain, a co-stimulatory domain, and a CD3 zeta domain.
128. The modified cell or the method of embodiments 99-127, wherein the first and second antigen binding domain is a Fab or a scFv.
129. The modified cell or the method of embodiments 99-128, wherein the modified cell is a T-cell, NK cell, macrophage, or dendritic cell.
130. The modified cell or the method of embodiments 99-128, wherein the modified cell is a T-cell.
131. The modified cell or the method of any one of embodiments 99-130 wherein the immune checkpoint molecule is selected from the group consisting of programmed death 1 (PD-1), cytotoxic T lymphocyte antigen-4 (CTLA-4), B- and T-lymphocyte attenuator (BTLA), T-cell immunoglobulin mucin-3 (TIM-3), lymphocyte-activation protein 3 (LAG-3), T-cell immunoreceptor with Ig and ITIM domains (TIGIT), leukocyte-associated immunoglobulin-like receptor 1 (LAIRI), natural killer cell receptor 2B4 (2B4), and CD 160.
132. The modified cell or the method of embodiment 131, wherein immune checkpoint molecule is modified PD-1.
133. The modified cell or the method of embodiment 131, wherein (i) the modified PD-1 lacks a functional PD-1 intracellular domain for PD-1 signal transduction, interferes with a pathway between PD-1 of a human T-cell and PD-L1 of a certain cell, comprises a PD-1 extracellular domain or a PD-1 transmembrane domain, or a combination thereof; or (ii) a modified PD-1 intracellular domain comprising a substitution or deletion as compared to a wild-type PD-1 intracellular domain, or comprises a soluble receptor comprising a PD-1 extracellular domain that binds PD-L1 of a certain cell.
134. The modified cell or the method of embodiment 131, wherein an inhibitory effect of PD-L1 on cytokine production of the T-cells of the population is less than an inhibitory effect of PD-L1 on cytokine production of T-cells that do not comprise at least a part of the nucleic acid sequence that encodes the modified PD-1.
135. The modified cell or the method of any one of embodiments 99-134, wherein the modified cell is engineered to express and secrete a therapeutic agent such as a cytokine.
136. The modified cell or the method of embodiment 135, wherein the therapeutic agent comprises IFN-γ.
137. The modified cell or the method of embodiment 135, wherein the therapeutic agent comprises: (i) IL-6, IFN-γ, or a combination thereof; or (ii) IL-17, CCL19, or a combination thereof.
138. The modified cell or the method of embodiment 135, wherein the therapeutic agent comprises IL-15, IL-12, or a combination thereof.
139. The modified cell or the method of embodiment 135, wherein the molecule or the therapeutic agent is or comprises a recombinant or native cytokine.
140. The modified cell or the method of embodiment 135, wherein the therapeutic agent comprises a FC fusion protein associated with a molecule.
141. The modified cell or the method of embodiment 135, wherein the molecule is or comprises IL-12, IL-15, IL-6 or IFN-γ.
142. The modified cell or the method of embodiment 135, wherein expression of the therapeutic agent and/or the immune checkpoint molecule is regulated by Hif1a, NFAT, FOXP3, and/or NFkB.
143. The modified cell or the method of any one of embodiments 99-142, wherein the modified cell comprises a first targeting vector and a second targeting vector, the first targeting vector comprising a nucleic acid sequence encoding a CAR binding a blood antigen and the therapeutic agent, and the second targeting vector comprises a nucleic acid sequence encoding a CAR biding solid tumor antigen and a dominant negative form of the immune checkpoint molecule.
144. The modified cell or the method of any one of embodiments 99-142, wherein the modified cell comprises a first targeting vector and a second targeting vector, the first targeting vector comprising a nucleic acid sequence encoding a CAR binding CD19 and the therapeutic agent, and the second targeting vector comprises a nucleic acid sequence encoding a CAR biding tMUC1 and a dominant negative form of PD-1.
145. The modified cell or the method of any one of embodiments 99-144, wherein the modified cell is derived form a healthy donor or the subject having the cancer.
146. The modified cell or the method of any one of embodiments 99-145, wherein the modified cell has a reduced expression of endogenous TRAC gene.
147. The modified cell or the method of any one of embodiments 99-146, wherein the modified cell comprises a first nucleic acid encoding a dominant negative form of an immune checkpoint molecule and a CAR binding a B cell antigen and a second nucleic acid encoding a CAR binding a solid tumor antigen.
148. The modified cell or the method of any one of embodiments 99-147, wherein the immune check point molecule is a dominant negative form of PD-1 (dnPD-1), the B cell antigen is CD19, and the solid tumor antigen is tMUC1.
149. The modified cell or the method of any one of embodiments 99-146, wherein the modified cell comprises a first nucleic acid encoding a CAR binding a B cell antigen and a second nucleic acid encoding a dominant negative form of an immune checkpoint molecule and a CAR binding a solid tumor antigen.
150. The modified cell or the method of any one of embodiments 99-146 or 149, wherein the immune check point molecule is a dominant negative form of PD-1 (dnPD-1), the B cell antigen is CD19, and the solid tumor antigen is tMUC1.
151. A method of obtaining a mix population of cells comprising culturing the modified cell of embodiment 148 to obtain a mix population of cells comprising CAR cells expressing a CD19, CAR cells expressing CD19 and dnPD-1, and CAR cells expressing tMUC1.
152. A mix population of cells comprising CAR cells expressing a CD19, CAR cells expressing CD19 and dnPD-1, and CAR cells expressing tMUC1.
153. The method of embodiment 151 or the mix population of cells of embodiment 152, wherein the CAR cells are CAR T cells.
154. A method of obtaining a mix population of cells comprising culturing the modified cell of embodiment 150 to obtain a mix population of cells comprising CAR cells expressing a CD19, CAR cells expressing tMUC1 and dnPD-1, and CAR cells expressing tMUC1.
155. A mix population of cells comprising CAR cells expressing a CD19, CAR cells expressing tMUC1 and dnPD-1, and CAR cells expressing tMUC1.
156. The method of embodiment 154 or the mix population of cells of embodiment 155, wherein the CAR cells are CAR T cells.
157. A method of treating cancer, wherein the method comprises administering the mix population of cells of any one of embodiments 151-156 to a subject diagnosed with cancer.

### EXAMPLES

### Example 1

### Expression of CAR and Modified PD-1 on Primary T-Cells and their Anti-Tumor Activity

Primary T-cells were obtained from patients. The obtained primary T-cells were transduced with lentiviral vectors. Flow-cytometry was performed to determine the expression of CAR and various modified PD-1 in primary T-cells. Techniques related to cell cultures, construction of lentiviral vectors, flow cytometry, and other related techniques are provided in U.S. Pat. No. 9,572,837, assigned to Innovative Cellular Therapeutics CO., LTD., and incorporated by reference in its entirety.

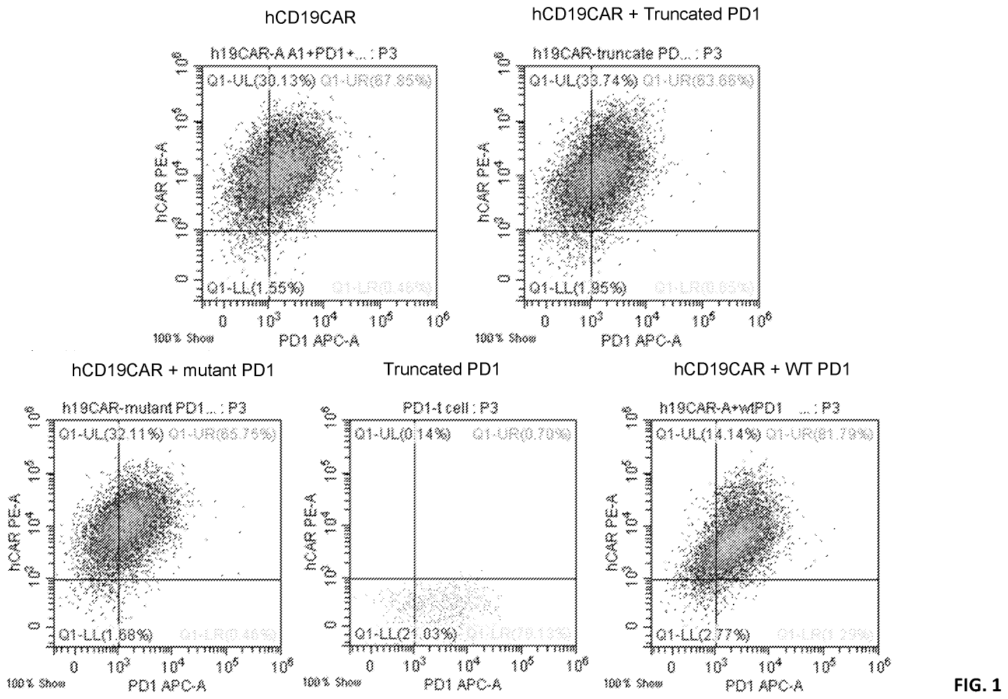

FIG. 1 shows flow cytometry results of modified PD-1 and CAR expression. DnPD-1 (PD-1 WT extracellular domain+PD-1 WT transmembrane domain+SEQ ID NO: 36).

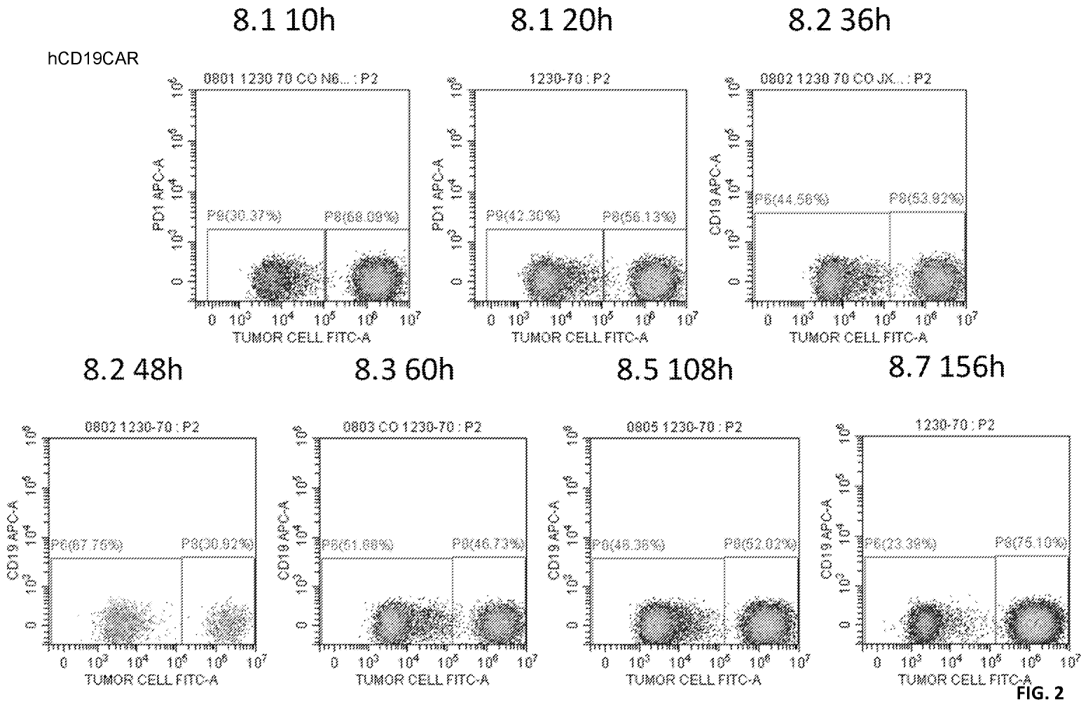

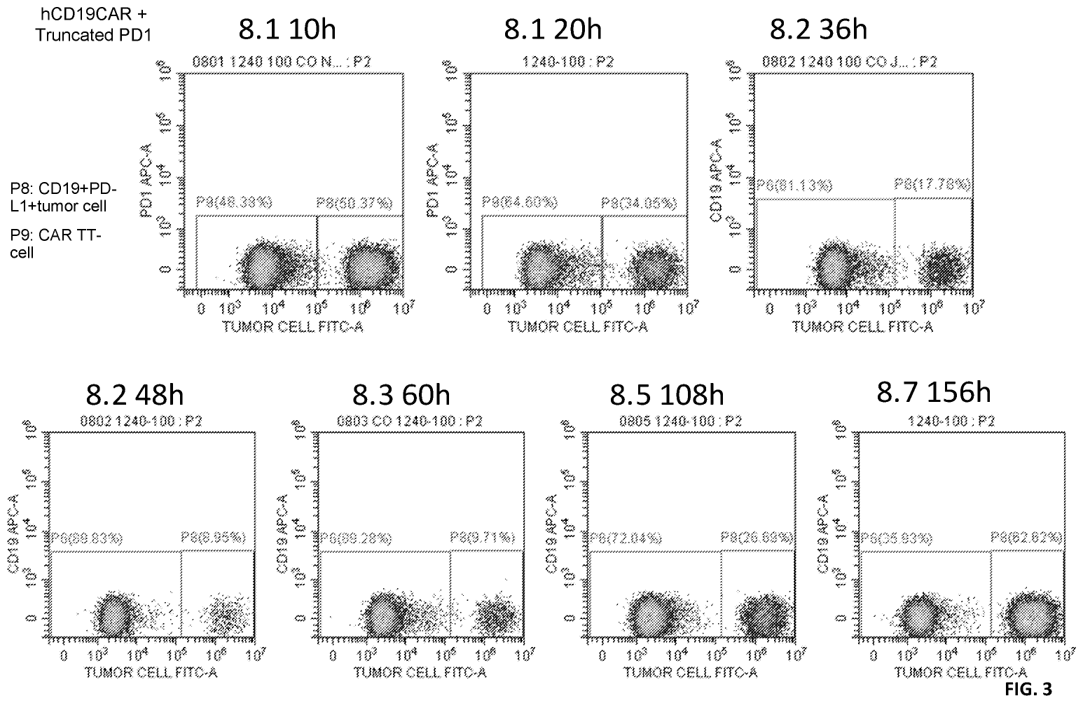

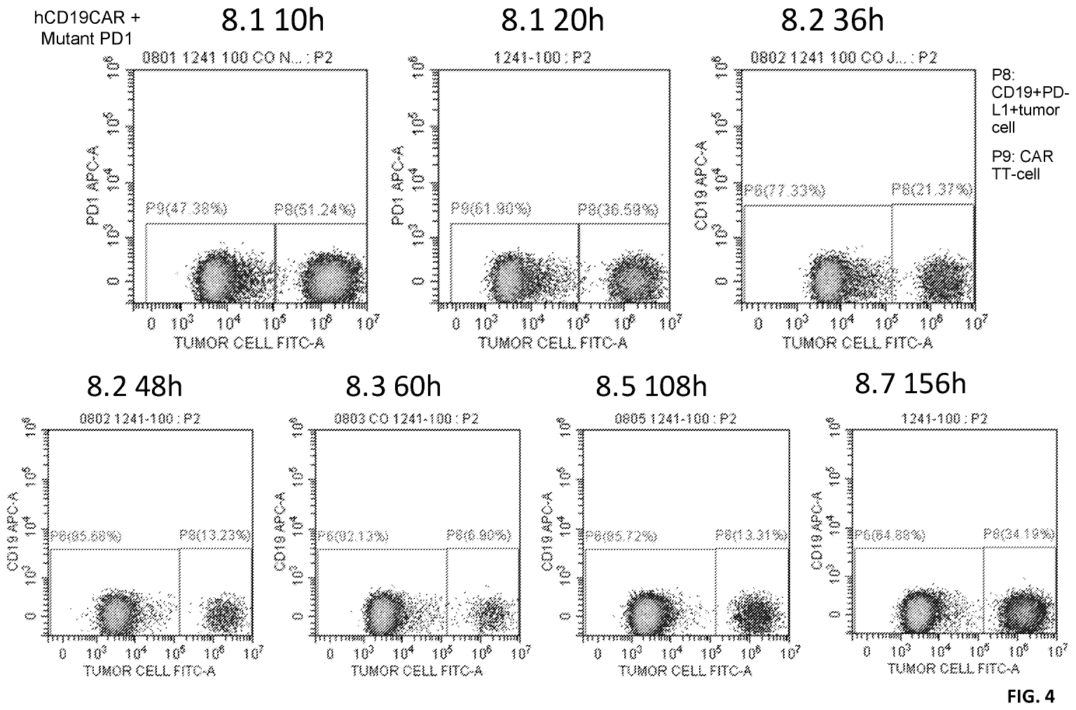

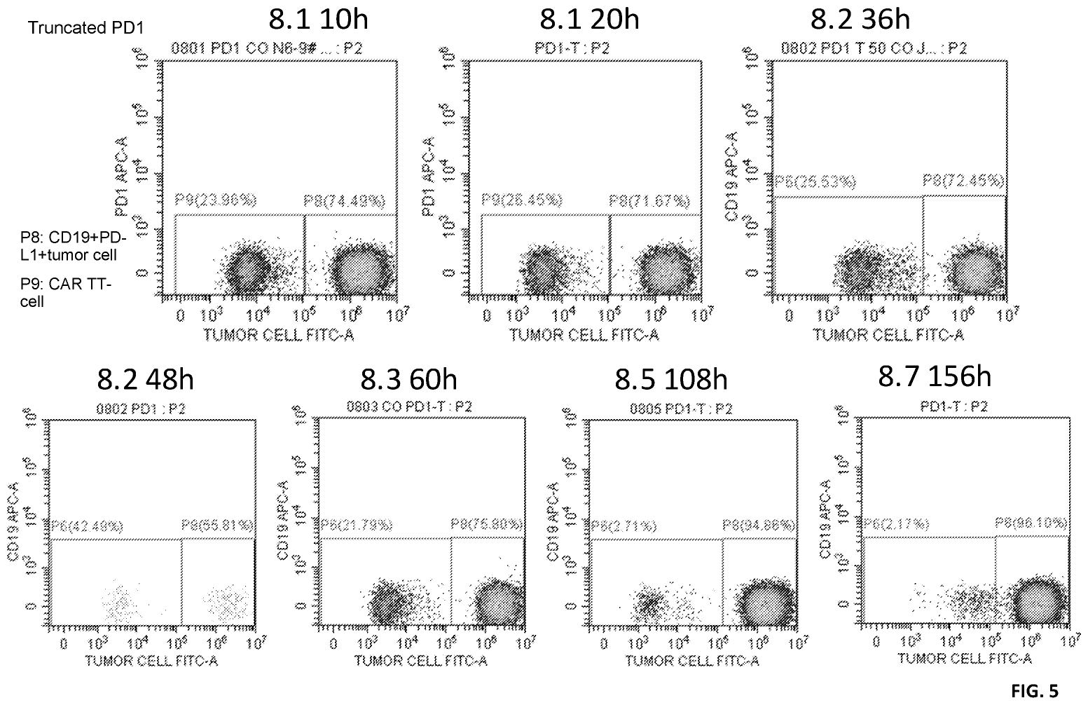

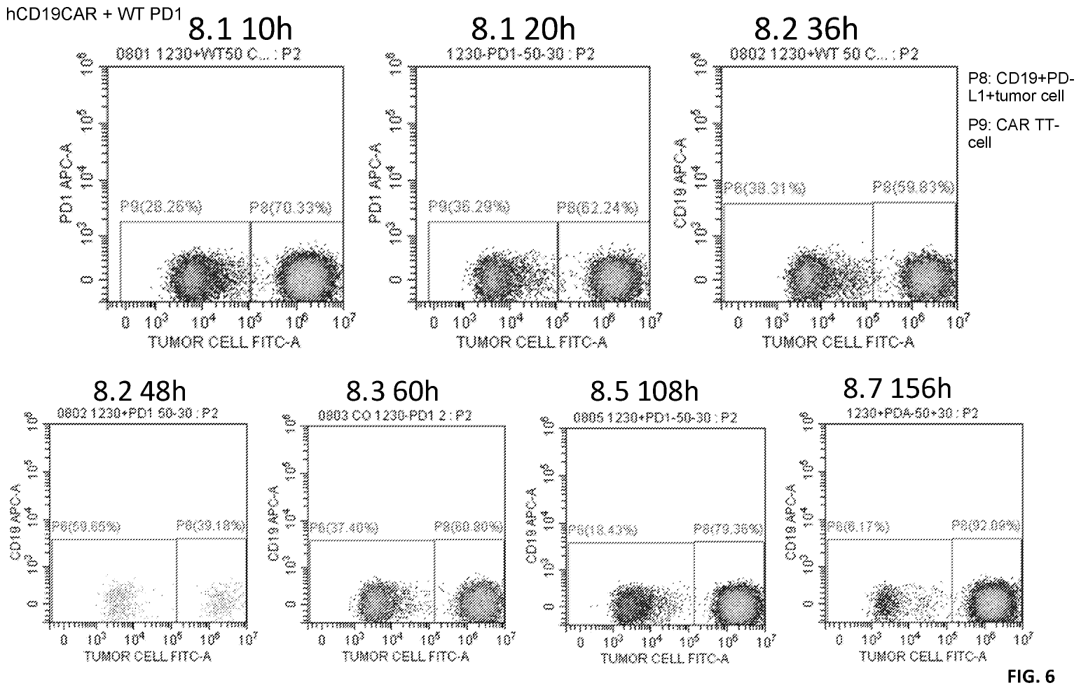

FIGS. 2-6 show the results of killing assay. Day 1: infection, and Day 3: co-culture. From the results, the anti-tumor ability of CAR comprising dnPD-1 was higher than that of the CAR without dnPD-1. At 60 h, only 8% of the tumors remained in sample cultured with CAR comprising dnPD-1, while 46.7% of tumors remained in sample cultured with CAR without dnPD-1. The negative control had 75.8% tumor. From 60 h to 156 h, tumor cells continued to grow; the mutant-dnPD-1 also exhibited anti-tumor capabilities, while CAR without dnPD-1 and negative controls did not have the anti-tumor capability. E:T (Effector versus. Target cells)=1:1; P8: CD19+PD-L1+ tumor cell; P9: CAR TT-cell. (Sequence Infor: CD19 CAR including SEQ ID NO: 5, 43, and 53; DnPD-1 including SEQ ID NO: 36 or 37). DnPD-1 (PD-1 WT extracellular domain+PD-1 WT transmembrane domain+SEQ ID NO: 36).

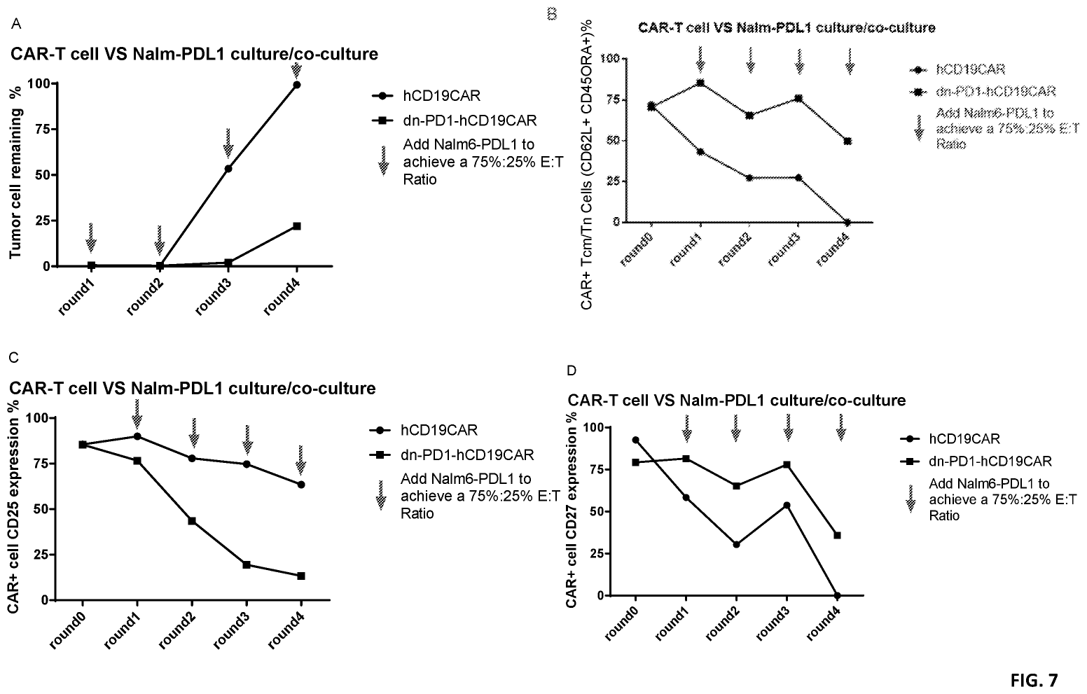

FIG. 7 shows the results of tumor challenging assay. A: CAR TT-cells were co-cultured with indicated tumor cells for multiple rounds. After 48 h-96 h of co-culture (one round), cells were analyzed by flow cytometry to confirm the percentage of tumor cells (GFP-tagged) as well as T-cells. Fresh tumor cells were added when the next round began. B: CAR TT-cells were co-cultured with tumor cells for multiple rounds. After 48 h-96 h of co-culture (one round), the cells were analyzed by flow cytometry to confirm the percentage of Tcm and Tn cells in CAR+ cells. Tcm and Tn cells were detected by the presence of CD62L+ and CD45RORA+ markers. Fresh tumor cells were added when the next round began. At the same time after co-culture, more dnPD-1 CAR TT-cells became Tcm cells than those found in h19CAR T-cells without dnPD-1. The hypothesis is that when the Tcm cells meet tumor cells, they become Teff cells quickly to kill tumor cells. However, in this case more, Tcm cells remained as Tcm cells. The persistence of dnPD-1 CAR T-cells is better than h19CAR T-cells. C: CAR TT-cells were co-cultured with tumor cells for multiple rounds. After 48 h-96 h of co-culture (one round), the cells were analyzed by flow cytometry to confirm the percentage of CAR+ T-cell CD25 expression. And fresh tumor cells were added when the next round began. CD25 is used to detect CAR TT-cell activation levels. The higher the CAR T-cell activation level, the higher the CD25 expression, and the less ability they have to continue killing and persist. D: CAR TT-cells were co-cultured with tumor cells for multiple rounds. After 48 h-96 h of co-culture (one round), the cells were analyzed by flow cytometry to confirm the percentage of CAR+ T-cell CD27 expression. Fresh tumor cells were added when the next round began. CD27 is used to detect CAR TT-cell activation and memory levels. The higher CAR TT-cells activation level, the lower the CD27 expression, and the less the ability they have to continue killing and persist. The higher the CAR TT-cells memory level, the higher the CD27 expression, and the more ability they have to continue killing and persist. (Sequence Infor: CD19 CAR including SEQ ID NO: 5, 43, and 53; DnPD1 including SEQ ID NO: 36 or 37). The results showed that dnPD-1 CAR T-cells persist with Tcm phenotypes, constituting essential preconditions for treatment efficacy. DnPD-1 (PD-1 WT extracellular domain+PD-1 WT transmembrane domain+SEQ ID NO: 36).

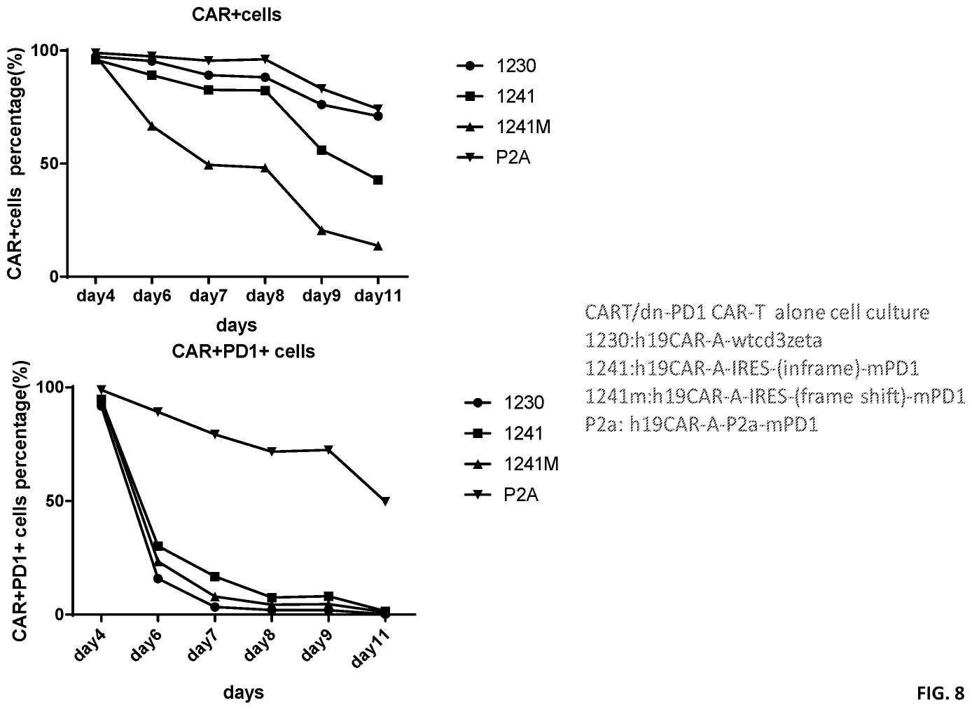

FIG. 8 shows CAR T-cell type analysis when they were cultured alone. (CD19 CAR: SEQ ID NO: 5 and DnPD1: SEQ ID NO: 36). DnPD-1 (PD-1 WT extracellular domain+PD-1 WT transmembrane domain+SEQ ID NO: 36).

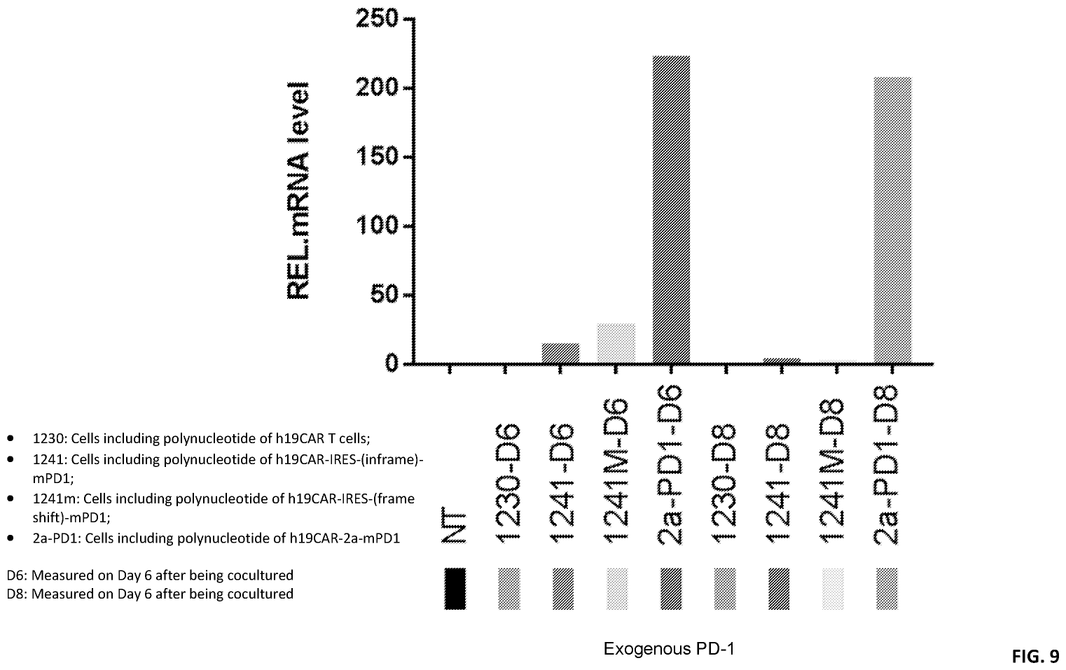

FIG. 9 discloses a histogram showing exogenous PD-1 mRNA levels in various CAR T-cells. DnPD-1 (PD-1 WT extracellular domain+PD-1 WT transmembrane domain+SEQ ID NO: 36). (Sequence Infor: CD19 CAR including SEQ ID NO: 5, 43, and 53; DnPD1 including SEQ ID NO: 36 or 37)

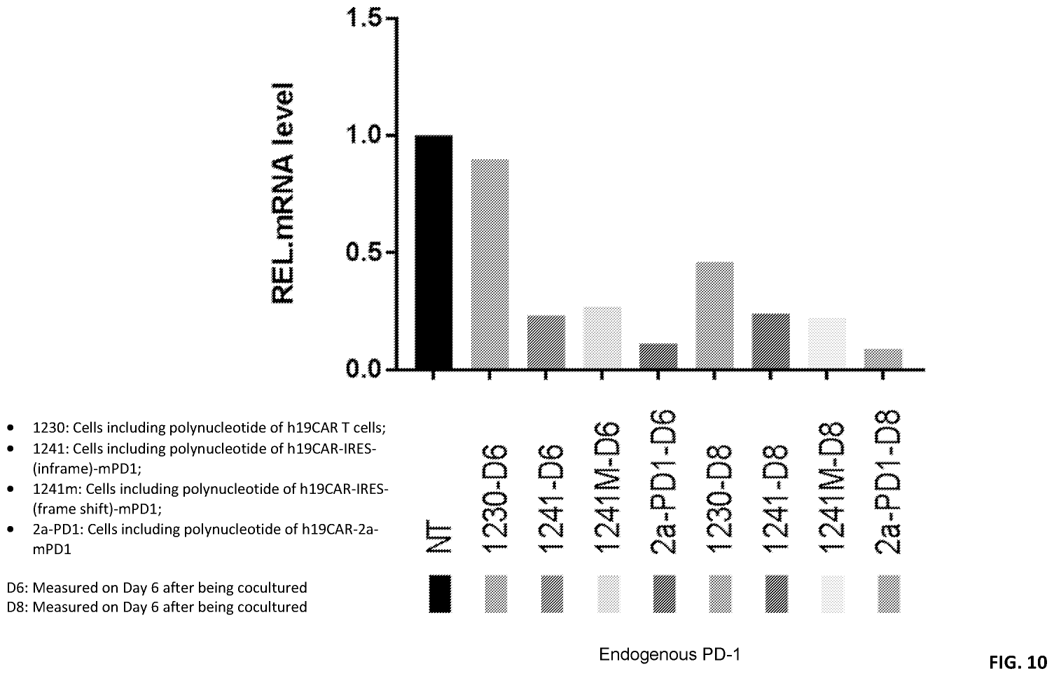

FIG. 10 discloses a histogram showing endogenous PD-1 mRNA levels in various CAR T-cells. DnPD-1 (PD-1 WT extracellular domain+PD-1 WT transmembrane domain+SEQ ID NO: 36). (Sequence Infor: CD19 CAR including SEQ ID NO: 5, 43, and 53; DnPD1 including SEQ ID NO: 36 or 37). DnPD-1 (PD-1 WT extracellular domain+PD-1 WT transmembrane domain+SEQ ID NO: 36).

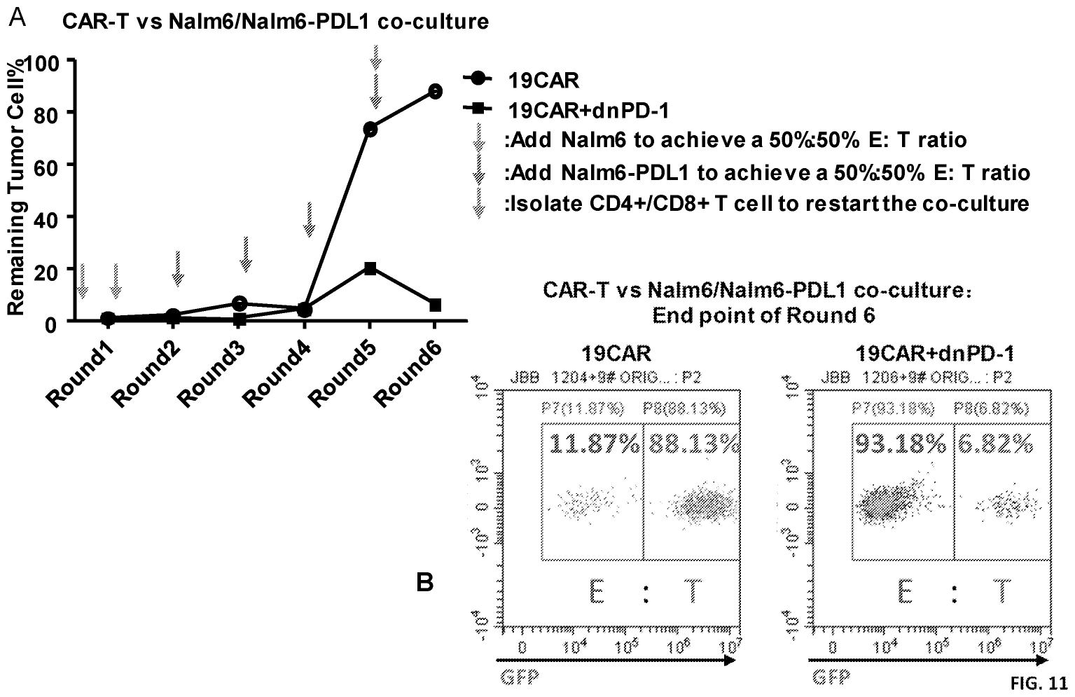

FIGS. 11A and 11B show results of tumor challenging assay showing 19CAR+dnPD-1 T-cells are more persistent than 19CAR TT-cells. A: CAR TT-cells were co-cultured with tumor cells for multiple rounds. After 2-10 days of co-culture (one round), cells were analyzed by flow cytometry to confirm the percentage of tumor cells (GFP-tagged) as well as T-cells. Fresh tumor cells were added when the next round began. B: After multiple rounds of co-culture, cells were analyzed by the end of round 6 to confirm the percentage of Nalm6-PD-L1 tumor cells and T-cells. Nalm6 and Nalm6-PD-L1 cells were GFP-tagged so that they can be distinguished from T-cells by flow cytometry. (Sequence Infor: CD19 CAR including SEQ ID NO: 5, 43, and 53; DnPD1 including SEQ ID NO: 36 or 37). DnPD-1 (PD-1 WT extracellular domain+PD-1 WT transmembrane domain+SEQ ID NO: 36).

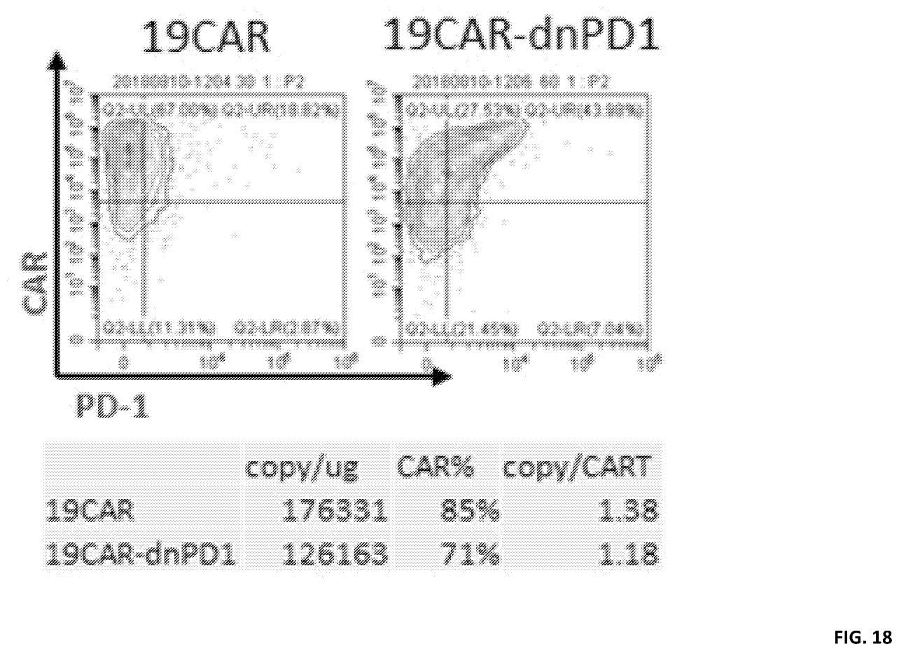

FIG. 18 shows CAR and PD-1 expression and copy numbers of anti-CD19 CAR and anti-CD19 DnPD-1 CAR TT-cells. Fresh T-cells were isolated from healthy volunteers (day 0). After stimulation by CD3/CD28 beads for 24 hours (day 1), the T-cells were transduced with 19CAR or 19CAR+dnPD-1 lentiviral vectors at multiplicity of infection (MOI) of 30 (anti-CD19 CAR) or 60 (anti-CD19 DnPD-1 CAR) for 24 hours (day 2). CAR and PD-1 expression were detected by flow cytometry. Copy numbers of integrated lentiviral vectors were measured by quantitative real-time PCR on day 10. DnPD-1 (PD-1 WT extracellular domain+PD-1 WT transmembrane domain+SEQ ID NO: 36).

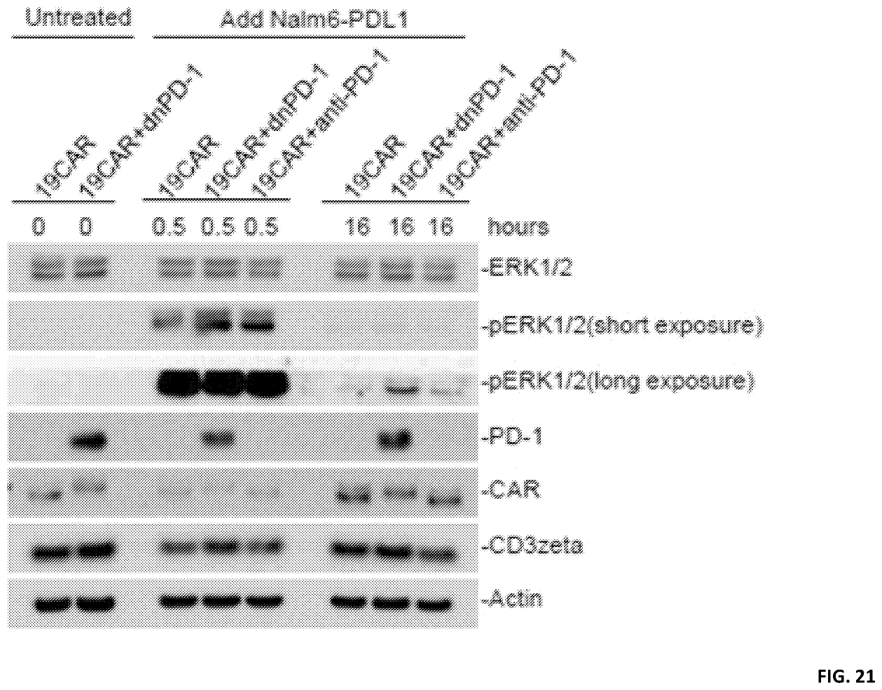

FIG. 21 shows expression of PD-1 of CAR T and dnPD-1 CAR T-cells on a western blot. A. To detect the expression of PD1, dnPD-1 CAR T cells were observed to have significant PD-1 expression (the expression of exogenous dnPD-1) compared to CAR T-cells (without dnPD-1). B. Detection of protection of endogenous signals (ERK1/2 phosphorylates after T-cell activation; PD-L1 action inhibits T-cell activated phosphorylation of ERK1/2). PD-L1 positive tumors were co-cultured with three groups of CAR T-cells, and T-cells were collected at 30 min and 16 h to detect the phosphorylation level of ERK1/2. The results showed that compared with the CAR T (i.e. 19CAR group without dnPD-1) and the CAR T (without dnPD-1) plus anti-PD1 drugs, dnPD-1 protected CAR T-cells (19CAR+dnPD-1 group, dnPD-1 CAR T-cells) showed higher phosphorylation at different time points. The level of chemistry shows its protection against T-cell signaling activation. The CART-cells were co-cultured to day 7-8, according to E:T=1:1, using Nalm6-PD-L1 (Nalm6 cell line overexpressing PD-L1, CAR % was adjusted before the experiment Level). DnPD-1 (PD-1 WT extracellular domain+PD-1 WT transmembrane domain+SEQ ID NO: 36).

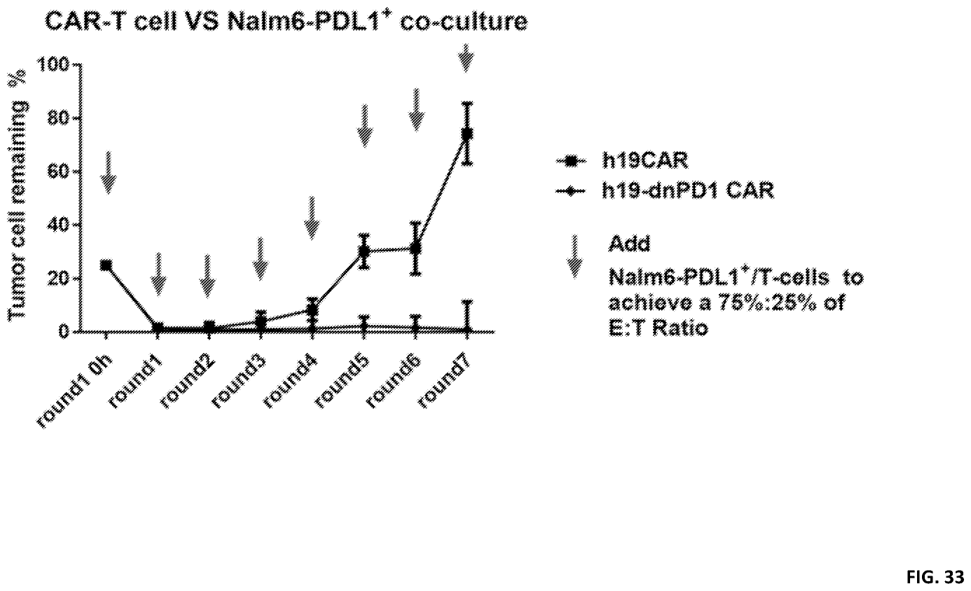

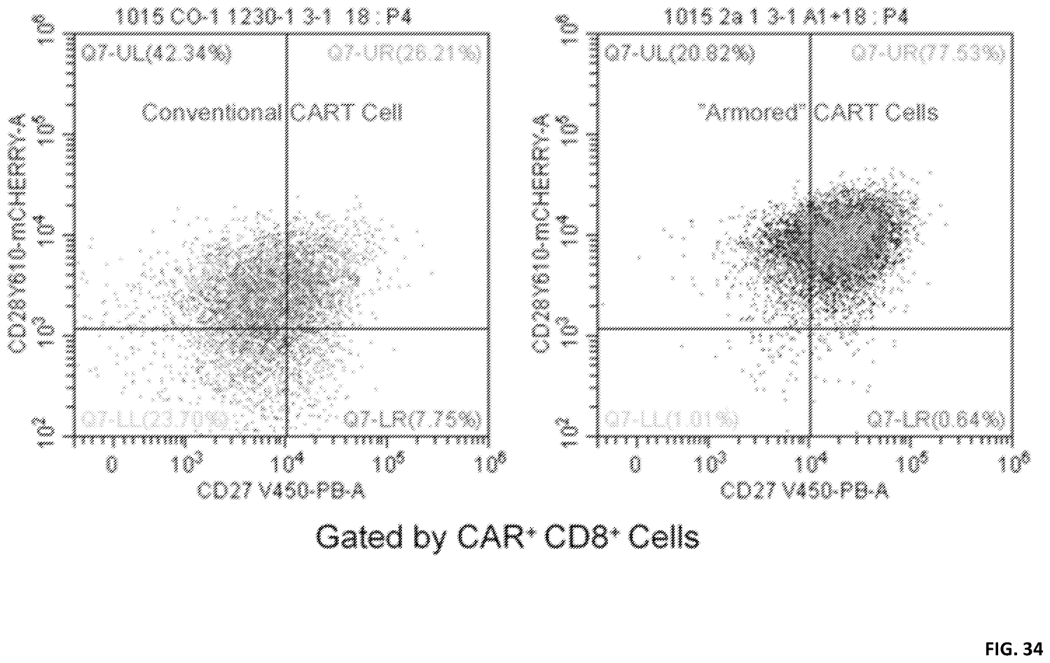

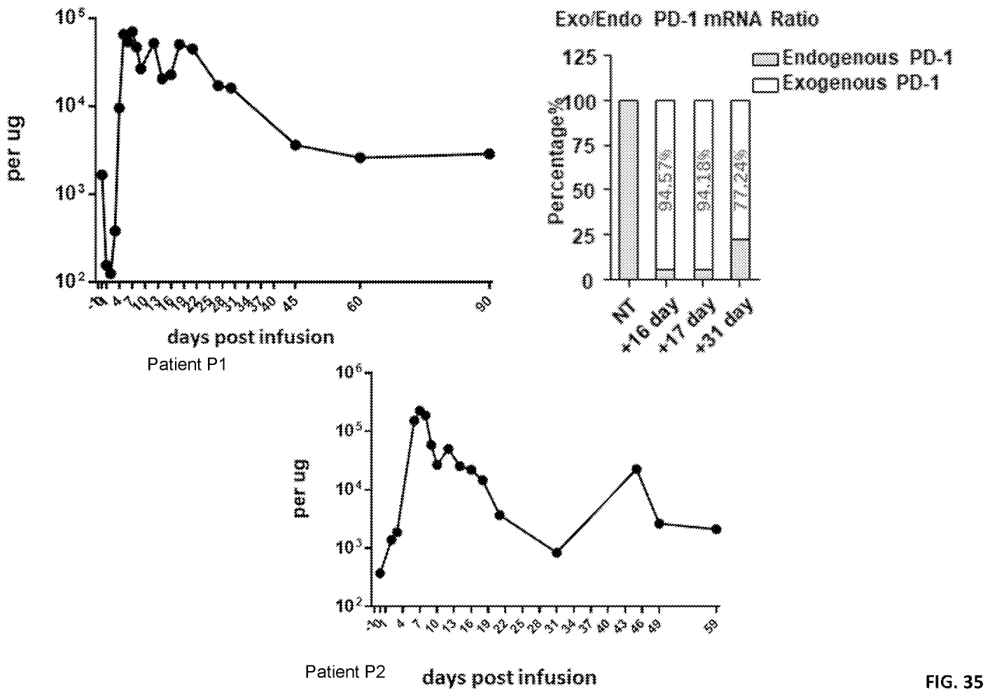

FIG. 34 shows flow cytometry results confirming that dnPD1 CAR T-cells exhibits enhanced ability of tumor killing after multiple-rounds of tumor challenge and more “memory-like” phenotypes. These results suggest dominant negative PD-1 (dnPD-1) molecules may protect CAR T-cells from exhaustion in the tumor microenvironment. CD27 and CD28 are markers of T-cell differentiation potential and memory-like characteristics and increased or continued expression indicates that the T cells have higher potential to continue to proliferate and be used as markers for determining levels of persistence of T-cell response. FIG. 34 shows the expression of two markers of CAR T-cells after multiple rounds of stimulation. It can be seen that dnPD-1 CAR cells have better CD27/CD28 expression relative to normal CAR T-cells (without dnPD-1), indicating that dnPD-1 CAR T-cells, after multiple-rounds of tumor challenges (See FIG. 33), maintain a high differentiation and proliferation ability. The results shown in FIGS. 33 and 34 indicate that dnPD1 may induce more memory-like CAR T-cells as compared to CAR T-cells (without dnPD-1) and these memory-like T-cells can contribute to persistent anti-tumor effect of dnPD1 CAR T-cells as compared to CAR T-cells (without dnPD-1).

### Example 2

### Cells Expressing Chimeric Receptors Establish Antitumor Effects in Patients with Relapsed/Refractory NHL (R/R Non-Hodgkin Lymphoma)

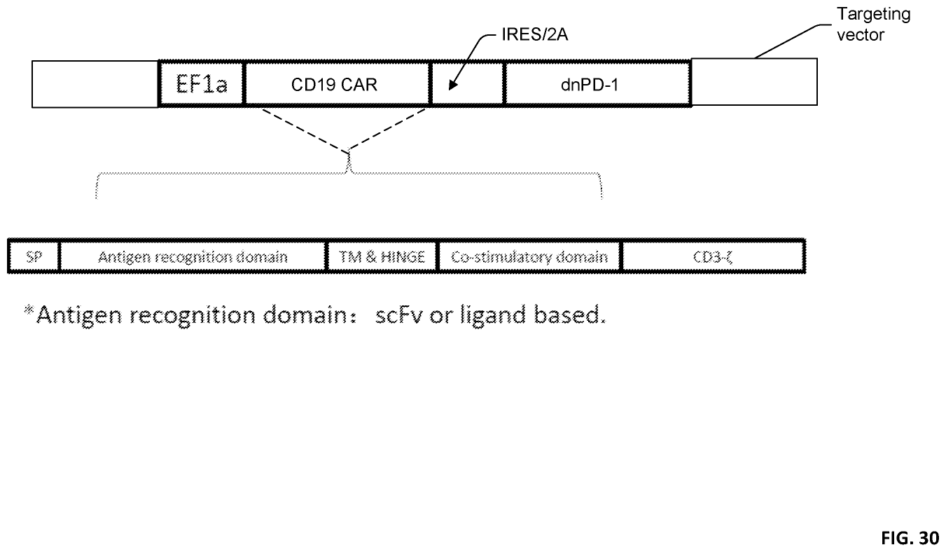

This clinical trial was designed to assess the safety and efficacy of infusing autologous T-cells modified to express humanized CD19 specific CAR/4-1BB/CD3-(and modified PD-1 (SEQ ID NO: 37 of which the intracellular domain comprises SEQ ID NO: 36) into the patients with R/R NHL (See FIG. 30). The inclusion criteria were as follows: 1) age not more than 60 years; 2) relapsed or refractory CD19+ NHL, and 3) measurable disease and adequate performance status and organ function. Patients with central nervous system leukemia (CNSL) were ineligible. The protocol was approved by hospitals and their Institutional Review Boards. All patients were provided written informed consent.

Prior to CD19CAR T-cell infusion, FACS analysis of transduction efficiency and in vitro cytotoxicity assays of CD19CAR T-cells were performed for each patient as described herein. Additionally, CD19CAR T-cell cultures were checked twice for possible contaminations by fungus, bacteria, mycoplasma, chlamydia, and endotoxin. The levels of cytokines IFN-γ, TNF-α, IL-4, IL-6, IL-10, IL-17, etc. in serum and of CSF were measured in a multiplex format according to the manufacturer's instructions.

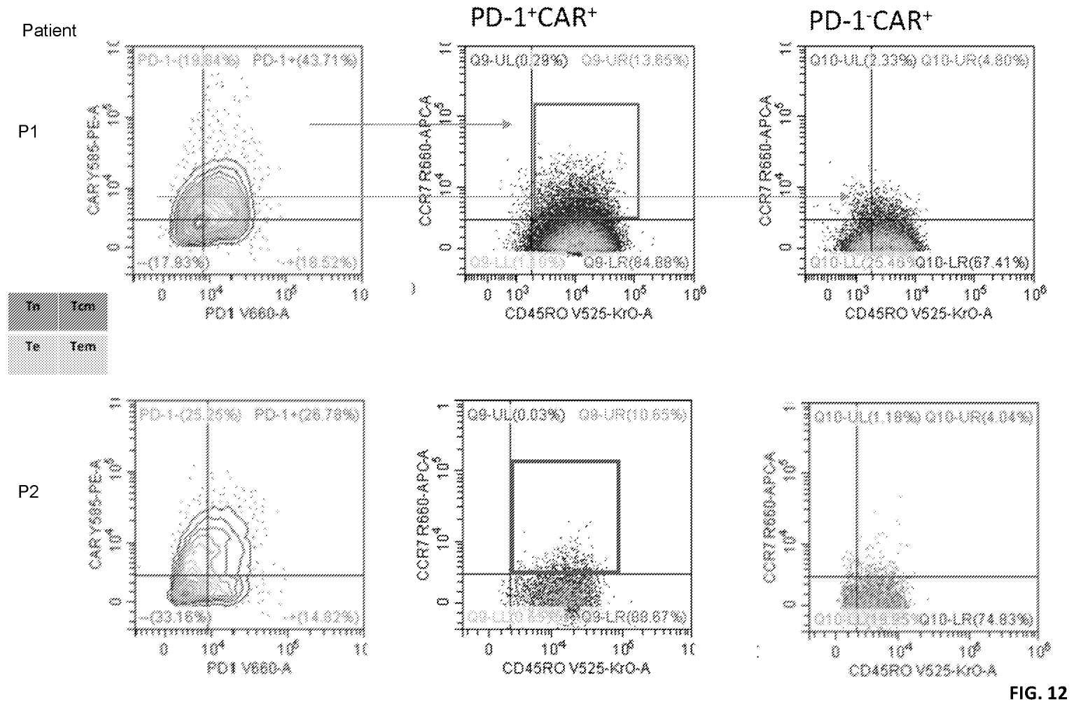

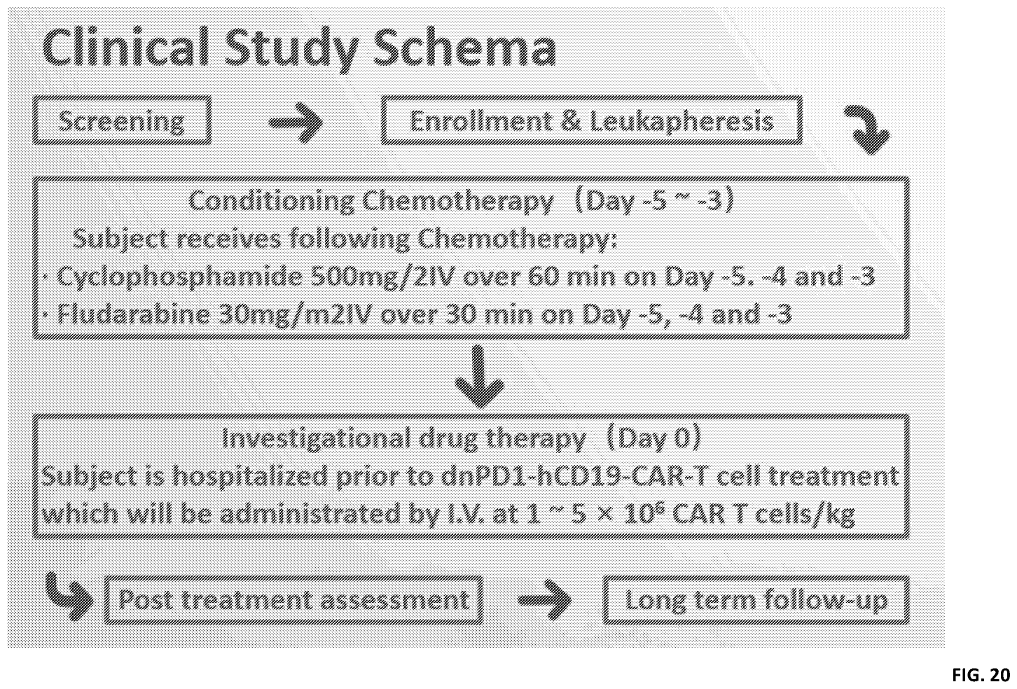

Three patients (P1, P2, and P3) with R/R NHL were treated with dnPD-1/CD19CAR T-cells according to the scheme shown in FIG. 20. Patients of Group 1 (four patients) were treated with CD19CAR T-cells, and patients of Group 2 (three patients) were treated with PD-1-CD19CAR T-cells. PET-CT Scanning results are provided in FIGS. 12 (P1 and P2), 13 and 19. These results demonstrate that T-cells expressing CD19CAR or PD-1/CD19CAR established antitumor effects in patients with R/R NHL. Further, these results demonstrate that patients treated with T-cells expressing dnPD-1/CD19 CAR showed complete remission (CR) or reduced tumor growth.

FIG. 12 shows results of CAR T-cell phenotype analysis on two patients (P1 and P2). dnPD-1 CAR T vector was used to prepare dnPD-1 CAR T-cells from subjects (B lymphoma patients). After infusion, changes in CAR T-cells in peripheral blood were detected and tumor volume changes were monitored. On the 7th day after the reinfusion, peripheral blood samples of the subjects were analyzed and CD8-positive CAR T-cell phenotype was observed. CAR/PD-1 was used to divide CD8-positive cells into four groups, and phenotypic analysis of CAR+PD-1positive and CAR+PD-1negative cells. The results showed that CAR+PD-1positive cells showed more Tcm cell ratio relative to CAR+PD-1 negative cells. (CCR7+CD45RO+ cells identified by red boxes in the figure.) This indicates that dnPD-1 helps maintain the CAR T-cell memory phenotype. (Sequence Infor: CD19 CAR including SEQ ID NO: 5, 43, and 53; DnPD1 including SEQ ID NO: 36 or 37). DnPD-1 (PD-1 WT extracellular domain+PD-1 WT transmembrane domain+SEQ ID NO: 36); P1, female, 57y, diagnosis of DLBCL, 7 cycles chemotherapy (4 times R-CHOP and 3 times R-ESHAP), never achieved CR before enrollment in this clinical trial. Conditioning Chemotherapy (Day −5 to day −3): Fludarabine 30 mg/m2/day, Cyclophosphamide 500 mg/m2/day; P2: Female, 61y, diagnosis of DLBCL in December 2017, 6 cycles chemotherapy (4 times R-CHOP and 2 times R-GemOx) and 25 times radiotherapy, never achieved CR before enrollment in this clinical trial.).

FIG. 13 shows PET-Scan results of two patients received the treatment of hCD19CAR+dnPD-1 T-cells. (Sequence Infor: CD19 CAR including SEQ ID NO: 5, 43, and 53; DnPD1 including SEQ ID NO: 36 or 37).

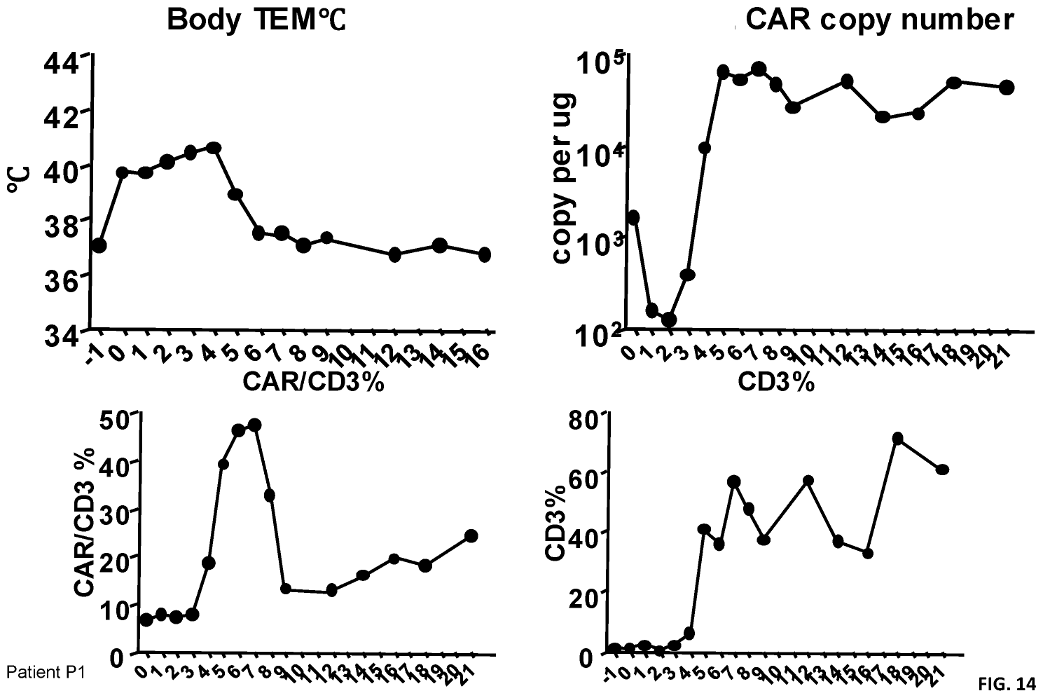

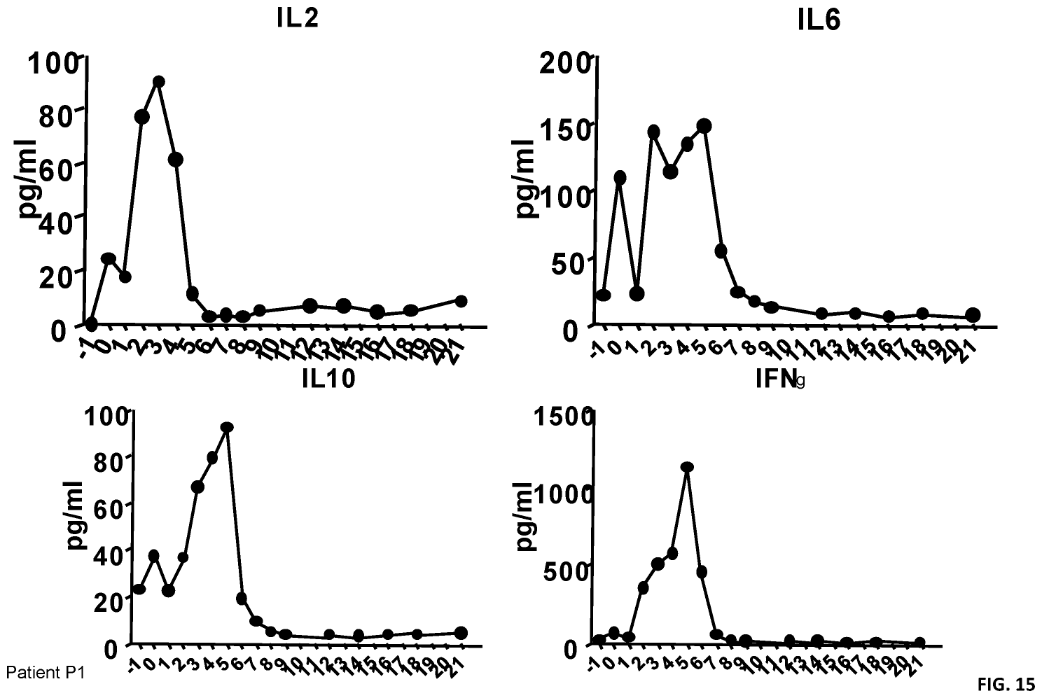

FIGS. 14 and 15 show results of cell analysis after CAR T-cell infusion of patient 1. Female, 57y, diagnosis of DLBCL in November 2017, 7 cycles chemotherapy (4 times R-CHOP and 3 times R-ESHAP), never achieved CR before enrollment in this clinical trial. (Sequence Infor: CD19 CAR including SEQ ID NO: 5, 43, and 53; DnPD1 including SEQ ID NO: 36 or 37) DnPD-1 (PD-1 WT extracellular domain+PD-1 WT transmembrane domain+SEQ ID NO: 36).

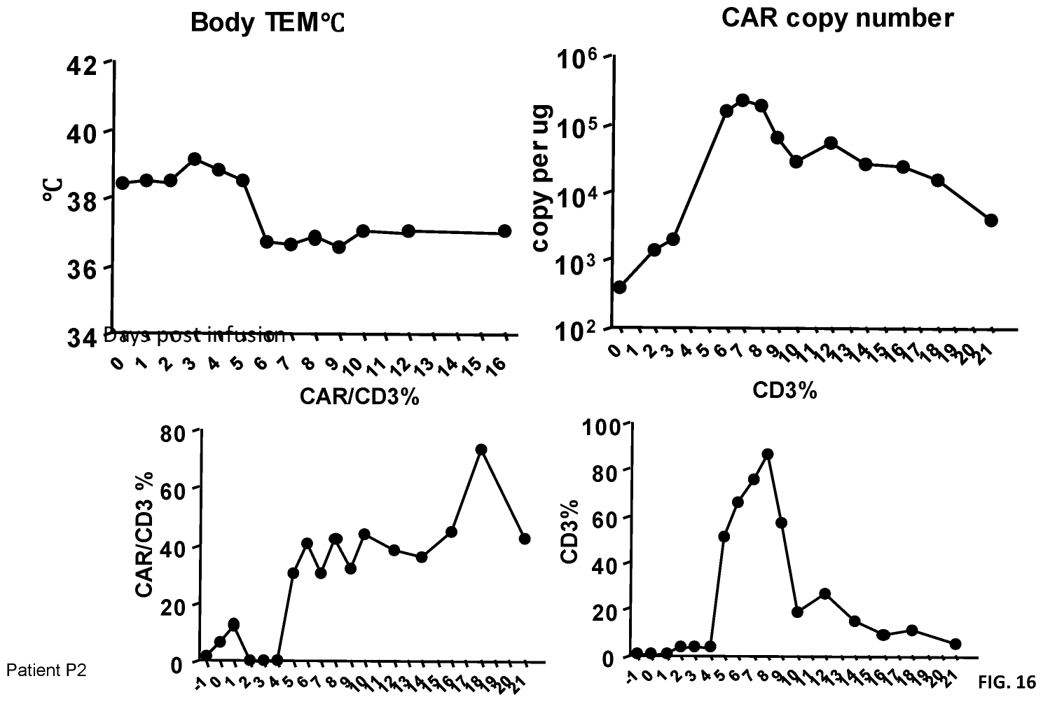

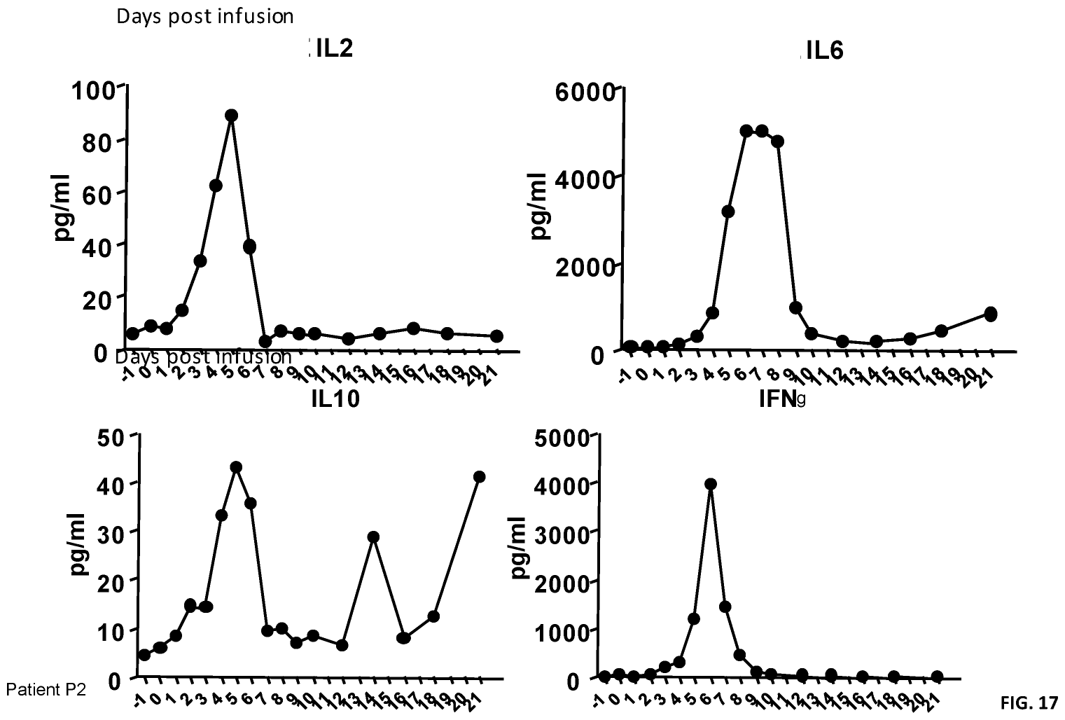

FIGS. 16 and 17 show results of cell analysis after CAR T-cell infusion of Patient 2. Female, 61y, diagnosis of DLBCL in December 2017, 6 cycles chemotherapy (4 times R-CHOP and 2 times R-GemOx) and 25 times radiotherapy, never achieved CR before enrollment in this clinical trial. (Sequence Infor: CD19 CAR including SEQ ID NO: 5, 43, and 53; DnPD1 including SEQ ID NO: 36 or 37) DnPD-1 (PD-1 WT extracellular domain+PD-1 WT transmembrane domain+SEQ ID NO: 36).

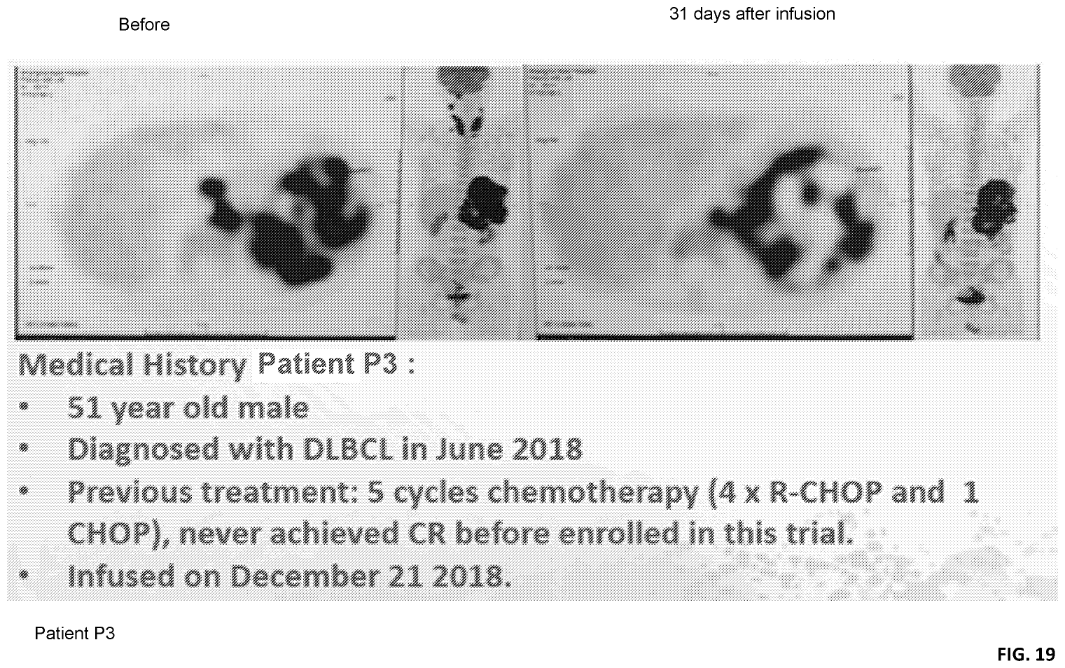

FIG. 19 provides information of a third patient (P3) and the Pet-scan results of the patient at 31 days after infusion. Patient is infused with DnPD-1 (PD-1 WT extracellular domain+PD-1 WT transmembrane domain+SEQ ID NO: 36).

FIG. 20 shows the clinical study scheme for the clinical trials in FIGS. 14-17 and 19.

### Example 3

### Nalm6-dnPD-1 and T-Cell Types

In vivo experiments were designed to measure T-cell type differences between CAR T-cells and dnPD-1 CAR T-cells. Experimental animals included 10 NPG mice, and experimental cells included Nalm6 leukemia cell line: 1×105 and 1×106 twice from tail vein of mice. CAR T-cells included normal-CAR T-cells (without dnPD-1), dnPD-1-CAR T-cells. Regents included 1 ml insulin syringe, blood collection needle

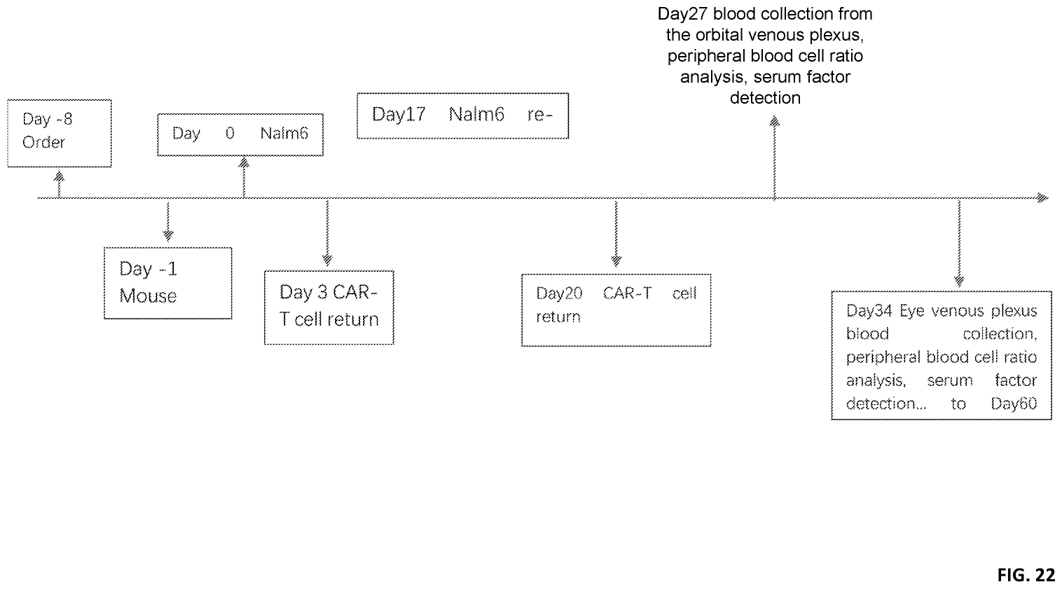

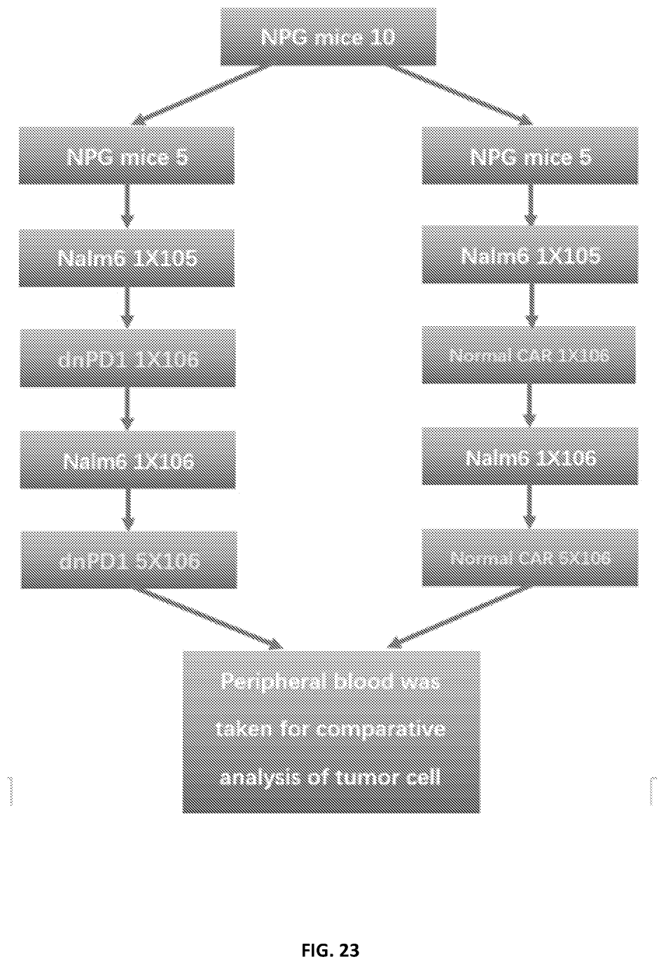

Experimental grouping and process are shown in FIGS. 22 and 23. Cell dosages are provided in the table below.

TABLE 2

| | Injection | Mouse | Theoretical cell | Total theoretical cell |
|---|---|---|---|---|
| Cell name | volume/only | only | dosage (CAR+) | dose (CAR+) |
| Nalm6 | 1 × 10⁵ | 10 | 1 × 10⁶ | 1.1 × 10⁷ |
| | 1 × 10⁶ | 10 | 1 × 10⁷ | |
| DnPD-1 | 1 × 10⁶ | 5 | 5 × 10⁶ | 3 × 10⁷ |
| CAR | 5 × 10⁶ | 5 | 2.5 × 10⁷ | |
| Normal | 1 × 10⁶ | 5 | 5 × 10⁶ | 3 × 10⁷ |
| CAR | 5 × 10⁶ | 5 | 2.5 × 10⁷ | |

The experimental development time was determined based on the cell preparation. The experimental cycle takes approximately 60 days, and tumor cells were injected at Day 0. After the CAR T-cells were returned to the mice, the mice were observed every 2-3 days and recorded.

### Example 4

### T-Cells Expressing DnPD-1/CD19&tMUC1 CAR Establish T-Cell Expansion and Cytokine Release in a Patient Having Breast Cancer

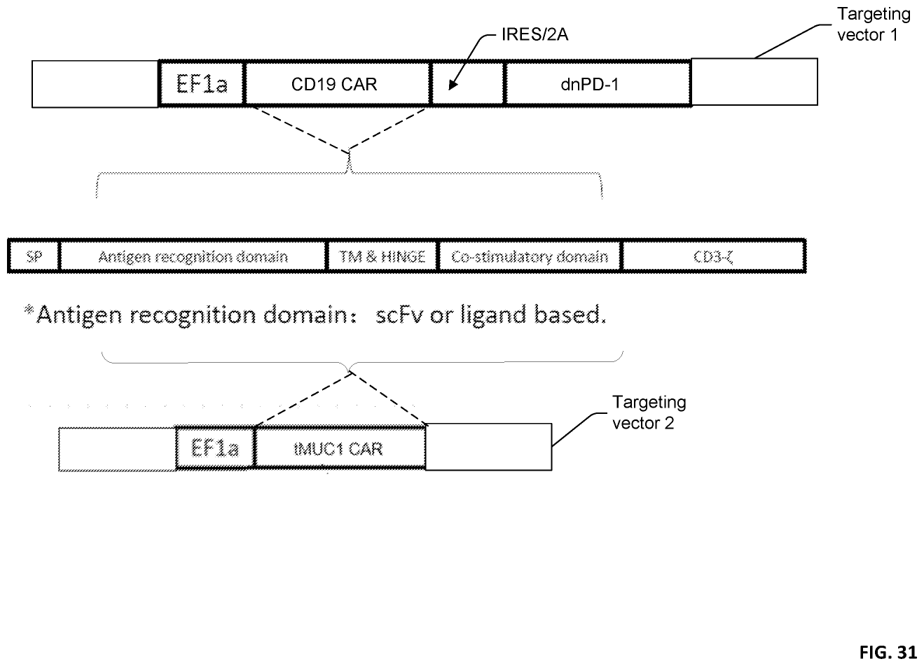

This clinical trial was designed to assess the safety and efficacy of infusing autologous T-cells modified to express humanized CD19 specific CAR/4-1BB/CD3-(as well as modified PD-1 (SEQ ID NO: 37 of which the intracellular domain comprises SEQ ID NO: 36), and/or tMUC1 specific CAR/4-1 BB/CD3-((SEQ ID NO: 21) into the patients with breast cancer (See FIG. 31). The inclusion criteria were as follows: 1) age not more than 65 years; 2) relapsed or refractory breast cancer; and 3) measurable disease and adequate performance status and organ function. Patients with central nervous system leukemia (CNSL) were ineligible. The protocol was approved by hospitals and their Institutional Review Boards. The patients were provided written, informed consent.

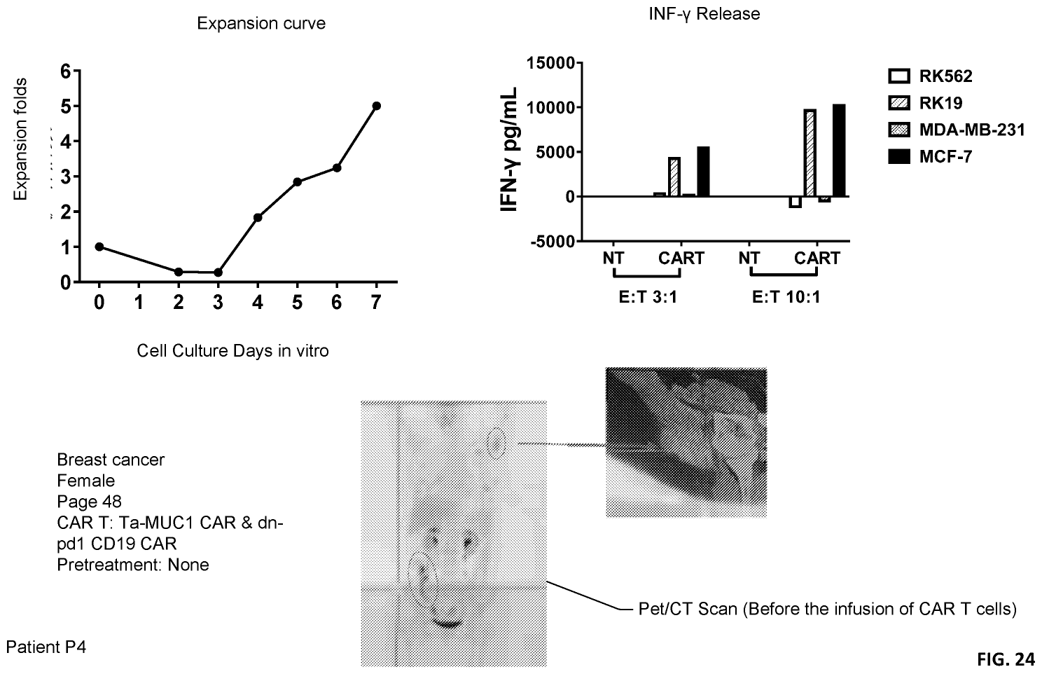

Peripheral blood mononuclear cells (PBMCs) were obtained from patient (P4) and cultured using TEXMACS culture containing IL-2. CD4 and CD8 magnetic beads were used to sort and select T-cells in the PBMCs. The appropriate amount of the starting culture was selected and Transact activator was used to activate T-cells. MACS® GMP T Cell TransAct™ includes a colloidal polymeric nanomatrix covalently attached to humanized recombinant agonists against human CD3 and CD28. The nanomatrix MACS GMP T Cell TransAct can be sterile filtered, and excess reagent can be removed by centrifugation and following conventional supernatant replacement or simply by medium wash. This reagent is suitable for the use in automated culture systems, such as the CliniMACS Prodigy® Instrument. The cell number of Group 3 was 9.1×107. The number of corresponding carriers and the volume of the carrier were calculated according to the required carrier MOI. The tMUC1-CAR vector MOI (SEQ ID NO: 21) was 30:1, and the MOI of dnPD-1/CD19CAR (dnPD-1: SEQ ID NO: 37; scFv of humanized CD19: SEQ ID NO: 5) vector was 5:1. The CAR T-cells were culture, and T-cell expansion and IFN-γ release were measured (FIG. 24). IFN-γ release in co-cultivation assays was performed to test T-cell killing functions. CAR T-cells and different single type or multiple types of substrate cells were co-cultured and the release of IFN-γ was observed. Substrate cells include MUC1-positive tumor cells (MCF-7), MUC1-negative tumor cells (231), and CD19-positive tumor cells (RK19). RK562 is a CD19negative cell, RK19 is a CD19 positive cell, MDA-MB-231 is a Mucl-negative cell, and MCF-7 is a Muc1-positive cell. Various CAR T-cells were obtained three days after infection. Beads were removed from the culture media, and CAR T-cells were resuspended. A ratio of E:T (Effector Cell:Target Cell) 3:1 and 10:1 (i.e., CAR T-cells:target tumor cells) of CAR T-cells and target tumor cells were co-cultured for about 24 hours. Subsequently, the supernatant was collected, and the release of IFN-γ was measured. Various levels of IFN-γ release were observed when CAR T-cells and the substrate cells were co-cultured. As shown in FIG. 24, CAR T-cells of all these Groups released IFN-γ in response to co-culturing with CD19-positive tumor cells and MUC1-positive tumor cells, while the CAR T-cells of Group 3 released more IFN-γ in response to co-culturing with CD19-positive tumor cells and MUC1-positive tumor cells. Techniques related to cell cultures, construction of cytotoxic T-lymphocyte assay can be found in “Control of large, established tumor xenografts with genetically retargeted human T-cells containing CD28 and CD137 domains,” PNAS, Mar. 3, 2009, vol. 106 no. 9, 3360-3365, which is incorporated herein by reference in its entirety.

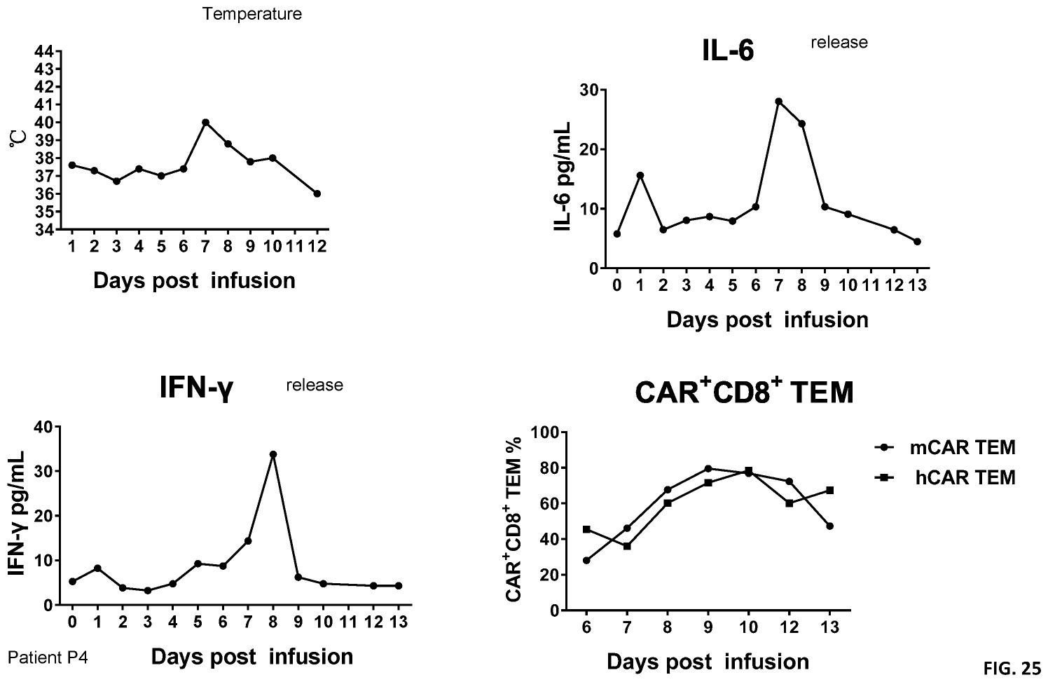

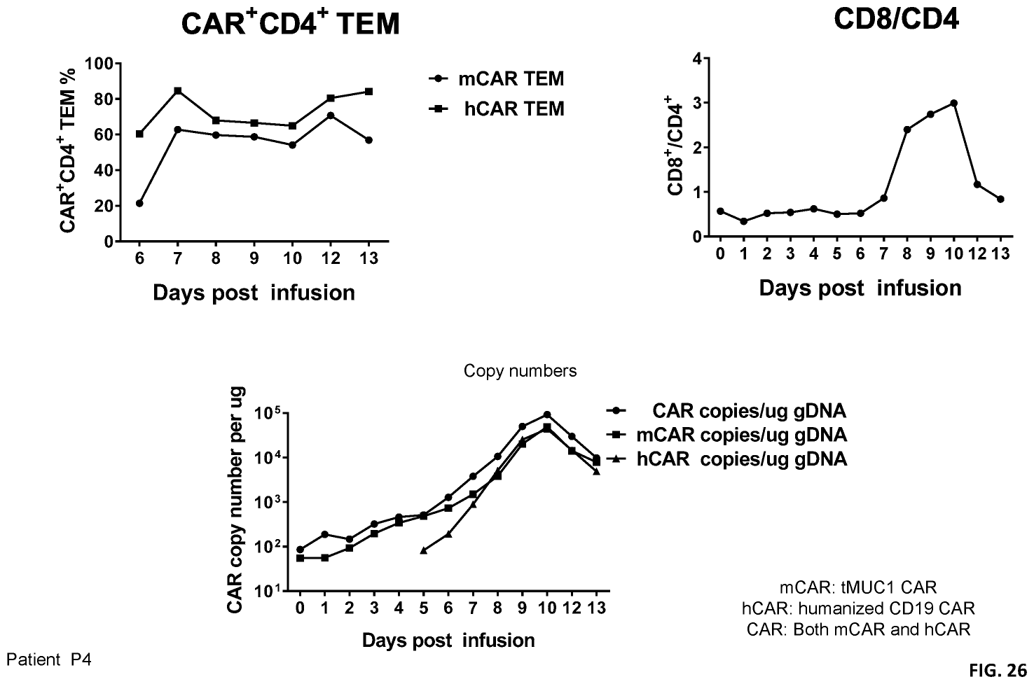

To enhance the efficacy of the CAR treatment, without chemotherapy pretreatment, the patient (P4) was treated with a mix population of CAR T-cells expressing CD19, CD19 and dnPD-1 (CD19/dnPD-1), and tMUC1 and CD19/dnPD-1 (1.03×107 CART/kg). The mix population of CAR T-cells includes CAR T-cells expressing CD19, CAR T-cells expressing CD19/dnPD-1, and CAR T-cells expressing both tMUC1 and CD19/dnPD-1. As shown in FIGS. 25 and 26, T-cell expansion, cytokine release, and other parameters were observed after infusion of the mix population of CAR T-cells. At 12 days after the infusion, it was observed the patient's armpits had signs of softening/shrinking.

TABLE 3

| SEQ | | SEQ | |
|---|---|---|---|
| ID NO: | Identity | ID NO: | Identity |
| 1 | SP | 30 | Tumor associated MUC1 scFv 1 |
| 2 | Hinge & transmembrane | 31 | Tumor associated MUC1 scFv-1 VH |
| | domain | | |
| 3 | Co-stimulatory region | 32 | Tumor associated MUC1 scFv-1 VL |
| 4 | CD3-zeta | 33 | Tumor associated MUC1 scFv 2 |
| 5 | scFV Humanized CD19 | 34 | Tumor associated MUC1 scFv2 VH |
| 6 | scFV CD19 | 35 | Tumor associated MUC1 scFv2 VL |
| 7 | scFv FZD10 | 36 | Modified PD-1 intracellular domain -1 (two |
| | | | tyrosine kinase mutations) |
| 8 | scFv TSHR | 37 | Modified PD-1 of SEQ ID NO: 36 |
| | | | (extracellular, transmembrane, and |
| | | | intracellular domains) |
| 9 | scFv PRLR | 38 | Modified PD-1 intracellular domain -2 |
| 10 | scFv Muc 17 | 39 | Modified PD-1 intracellular domain -3 |
| 11 | scFv GUCY2C | 40 | Modified PD-1 intracellular domain -4 |
| 12 | scFv CD207 | 41 | Modified PD-1 intracellular domain -5 |
| 13 | Prolactin (ligand) | 42 | Removed PD-1 intracellular domain -2 |
| 14 | scFv CD3 | 43 | A hinge |
| 15 | scFv CD4 | 44 | Seq1: WT |
| 16 | scFv CD4-2 | 45 | Seq2: Y201F |
| 17 | scFv CD5 | 46 | Seq3: Y218F |
| 18 | WTCD3zeta | 47 | Seq4: Y201F Y218F |
| 19 | WTCD3zeta-BCMACAR | 48 | Seq5: Truncated (delete internal 190-223) |
| | full length | | |
| 20 | BCMACAR | 49 | Seq6: Replace with CD8 transmembrane |
| | | | (delete 161-223, add CD8 transmembrane) |
| 21 | MUC1CAR | 50 | Seq7: L141A Y201F Y218F |
| 22 | m19CAR-IRES- | 51 | Seq8: Truncated (delete internal 190- |
| | MUC1CAR | | 223) + L141A |
| 23 | hCD19CAR-IRES- | 52 | Seq9: Replace with CD8 |
| | MUC1CAR | | transmembrane + L141A |
| 24 | hCD22CAR-IRES- | 53 | WT CD3 zeta aa |
| | MUC1CAR | | |
| 25 | BCMACAR-IRES- | 45 | Modified PD-1 (WT) |
| | MUC1CAR | | |
| 26 | mCD19CAR-2A- | 55 | Modified PD-1 (Point mutation 1) |
| | MUC1CAR | | |
| 27 | hCD19CAR-2A- | 56 | Modified PD-1 (point mutations 2 sites-2) |
| | MUC1CAR | | |
| 28 | hCD22CAR-2A- | 57 | Modified PD-1 (point mutations 2 sites-3) |
| | MUC1CAR | | |
| 29 | BCMA-2A-MUC1CAR | 58 | Modified PD-1 2 |
| 59 | Modified PD-1-truncated | 60 | GS linker |

All publications, patents and patent applications cited in this specification are incorporated herein by reference in their entireties as if each individual publication, patent or patent application were specifically and individually indicated to be incorporated by reference. While the foregoing has been described in terms of various embodiments, the skilled artisan will appreciate that various modifications, substitutions, omissions, and changes may be made without departing from the spirit thereof.

1. A method for increasing or extending persistence of a subpopulation of immune cells in a subject having cancer, the method comprising:
administering an effective amount of a composition comprising T-cells to the subject, the T-cells including a chimeric antigen receptor (CAR) and a modified PD-1, the modified PD-1 lacking a functional PD-1 intracellular domain; and
monitoring T-cell response in the subject;
wherein the T-cells comprise naive T-cells, stem cell memory T-cells, and/or central memory T-cells; and
wherein the number and/or ratio of the subpopulation of T-cells in the subject is increased as compared to the number and/or ratio of subpopulation of immune cells in a subject administered with corresponding T-cells that do not comprise the modified PD-1 or the persistence of the subpopulation of immune cells in the subject is increased or extended as compared to the persistence of the subpopulation of immune cells in a subject administered with corresponding T-cells that do not include the modified PD-1, thereby increasing or extending persistence of a subpopulation of immune cells infused into the subject having cancer.

2. The method of claim 1, wherein the monitoring the T-cell response in the subject comprises at least one of: detecting or measuring mRNA of the modified PD-1; measuring a number white blood cells; measuring a number of naive T-cells, stem cell memory T-cells, and central memory T-cells; measuring copy number of a CAR molecule; measuring a number of CD3 positive cells; measuring a number of T-cells expressing CAR; and measuring a level of one or more cytokines.

3. The method of claim 1, wherein the T-cells in the subject comprises more memory T-cells than T-cells in a subject administered with T-cells comprising the CAR that does not include the modified PD-1.

4. The method of claim 1, wherein (i) the modified PD-1 comprises an amino acid sequence of SEQ ID: 36-41 or 55-59; or (ii) the modified PD-1 do not include SEQ ID: 42.

5. The method of claim 1, wherein the T-cells comprise a dominant negative variant of PD-1, and endogenous gene of PD-1 of the T-cells is not disrupted.

6. The method of claim 1, wherein the CAR comprises an extracellular domain, a transmembrane domain, and an intracellular domain, wherein the extracellular domain binds an antigen.

7. The method of claim 6, wherein the extracellular domain comprises one or more amino acid sequences of SEQ ID: 5-17 or 30-35.

8. The method of claim 6, wherein the T-cells comprise one or more nucleic acid sequences of SEQ ID: 18-29.

9. The method of claim 6, wherein the intracellular domain comprises a co-stimulatory signaling region that comprises an intracellular domain of a co-stimulatory molecule selected from the group consisting of CD27, CD28, 4-1 BB, OX40, CD30, CD40, PD-1, ICOS, lymphocyte function-associated antigen-1 (LFA-1), CD2, CD7, LIGHT, NKG2C, B7-H3, and any combination thereof.

10. The method of claim 6, wherein the antigen is Epidermal growth factor receptor (EGFR), Variant III of the epidermal growth factor receptor (EGFRvIII), Human epidermal growth factor receptor 2 (HER2), Mesothelin (MSLN), Prostate-specific membrane antigen (PSMA), Carcinoembryonic antigen (CEA), Disialoganglioside 2 (GD2), Interleukin-13Ra2 (IL13Rα2), Glypican-3 (GPC3), Carbonic anhydrase IX (CAIX), L1 cell adhesion molecule (L1-CAM), Cancer antigen 125 (CA125), Cluster of differentiation 133 (CD133), Fibroblast activation protein (FAP), Cancer/testis antigen 1B (CTAG1B), Mucin 1 (MUC1), Folate receptor-α (FR-α), CD19, FZD10, TSHR, PRLR, Muc 17, GUCY2C, CD207, CD3, CD5, B-Cell Maturation Antigen (BCMA), or CD4.

11. The method of claim 1, wherein the composition comprises T-cells comprising a CAR binding to a solid tumor antigen and a CAR binding a white cell antigen.

12. The method of claim 11, wherein the white cell antigen is a B cell antigen.

13. The method of claim 12, wherein the solid tumor antigen is tMUC 1, PRLR, CLCA1, MUC12, GUCY2C, GPR35, CR1L, MUC 17, TMPRSS11B, MUC21, TMPRSS11E, CD207, SLC30A8, CFC1, SLC12A3, SSTR1, GPR27, FZD10, TSHR, SIGLEC15, SLC6A3, KISS1R, QRFPR, GPR119, CLDN6, UPK2, ADAM12, SLC45A3, ACPP, MUC21, MUC16, MS4A12, ALPP, CEA, EphA2, FAP, GPC3, IL13-Ra2, Mesothelin, PSMA, ROR1, VEGFR-II, GD2, FR-α, ErbB2, EpCAM, EGFRvIII, or EGFR.

14. The method of claim 12, wherein the B cell antigen is CD19, CD20, CD22, or BCMA.

15. The method of claim 1, wherein expression of the modified PD-1 is regulated by Hif1a, NFAT, FOXP3, and/or NFkB.

16. The method of claim 1, wherein the T-cells express and/or secrete a therapeutic agent comprising at least one of IFNγ, IL-15, or IL-12.


SEQUENCE LISTING (60 sequences)

SEQ ID NO: 1 - protein, 21 aa, Artificial Sequence
  Synthesized
MALPVTALLLPLALLLHAARP

SEQ ID NO: 2 - protein, 69 aa, Artificial Sequence
  Synthesized
AKPTTTPAPRPPTPAPTIASQPLSLRPEACRPAAGGAVHTRGLDFACDIYIWAPLAGTCGVLLLSLVIT

SEQ ID NO: 3 - protein, 42 aa, Artificial Sequence
  Synthesized
KRGRKKLLYIFKQPFMRPVQTTQEEDGCSCRFPEEEEGGCEL

SEQ ID NO: 4 - protein, 112 aa, Artificial Sequence
  Synthesized
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - protein, 242 aa, Artificial Sequence
  Synthesized
DIQMTQSPSSLSASVGDRVTITCRASQDISKYLNWYQQKPGKAPKLLIYHTSRLHSGVPSRFSGSGSGTDFTLTISSLQPEDFATYYCQQGNTLPYTFGQGTKVEIKGGGGSGGGGSGGGGSEVQLVESGGGLVQPGGSLRLSCAASGVSLPDYGVSWVRQAPGKGLEWVSVIWGSETTYYNSALKSRFTISRDNAKNSLYLQMNSLRAEDTAVYYCAKHYYYGGSYAMDYWGQGTLVTVSS

SEQ ID NO: 6 - protein, 242 aa, Artificial Sequence
  Synthesized
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 7 - protein, 237 aa, Artificial Sequence
  Synthesized
DIQMTQSPASLSVSVGETVTITCRASENIYSNLAWYQQKQGKSPQLLVYVATNLADGVPSRFSGSGSGTQYSLKINSLQSEDFGSYYCQHFWGTPYTFGGGTKLGGGGSGGGGSGGGGSEVQLQQSGAELVKPGASVKLSCTASGFNINDTYMHWVKQRPEQGLEWIGRIDPANGNTKYDPKFQGKATITADTSSNTAYLQLSSLTSEDTAVYYCARGARGSRFAYWGQGTLVTVSA

SEQ ID NO: 8 - protein, 244 aa, Artificial Sequence
  Synthesized
QSVLTQPPSVSAAPGQKVTISCSGSSSDIGSNYVSWYQQFPGTAPKLLIYDNNKRPSAIPDRFSGSKSGTSATLGITGLQTGDEADYYCGTWDSRLGIAVFGGGTQLTVLGGGGSGGGGSGGGGSEVQLVQSGAEVKKPGQSLKISCKASGYSLTDNWIGWVRQKPGKGLEWMGIIYPGDSDTRYSPSFQGQVTISADKSINTAYLQWSSLKASDTAIYYCVGLDWNYNPLRYWGPGTLVTVSS

SEQ ID NO: 9 - protein, 252 aa, Artificial Sequence
  Synthesized
DIVLTQSPASLAVSLGQGATISCRASKSVSTSGYTYMHWYQQKPGQPPKLLIYLASNLESGVPARFSGSGSGTDFTLNIHPVEEEDAATYYCQHSGELPPSFGGGTKLEIKRAGGGGSGGGGSGGGGSEVQLVESGGDLVKPGGSLKLSCAVSGFTFSSYGMSWVRQTPDKRLEWVATVSSGGTYTYYPDSVKGRFTISRDNAKNTLYLQMSSLKSEDSAMYYCARHRGNYYATYYYAMDYWGQGTSVTVSS

SEQ ID NO: 10 - protein, 243 aa, Artificial Sequence
  Synthesized
DVQITQSPSYLAASPGETITINCRASKSISKYLAWYQEKPGKTNKLLIYSGSTLQSGIPSRFSGSGSGTDFTLTISSLEPEDFAMYYCQQHHEYPYTFGGGTKLEIKGGGGSGGGGSGGGGSQVQLQQPGAELVRPGASVKLSCKASGYTFTTYWMNWVKQRPEQGLEWIGRIDPYDSETHYNQKFKDKAILTVDKSSSTAYMQLSSLTSEDSAVYYCARGPYYGTNPWFPYWGQGTLVTVSS

SEQ ID NO: 11 - protein, 241 aa, Artificial Sequence
  Synthesized
EIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIKGGGGSGGGGSGGGGSQVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSS

SEQ ID NO: 12 - protein, 246 aa, Artificial Sequence
  Synthesized
DVVMTQTPLSLPVRLGDQASISCRSSQSLVHSNGNTYLHWYLQKPGQSPKLLIYKVSNRFSGVPDRFSGSGSGTNFTLKISRVEAEDLGLYFCSQSTHVPYTFGGGTKLEIKRAGGGGSGGGGSGGGGSQVQLRQSGPELVKPGASVKMSCKASGYTFTDYVISWVKQRTGQGLEWIGDIYPGSGYSFYNENFKGKATLTADKSSTTAYMQLSSLTSEDSAVYFCATYYNYPFAYWGQGTLVTVSA

SEQ ID NO: 13 - protein, 199 aa, Artificial Sequence
  Synthesized
LPICPGGAARCQVTLRDLFDRAVVLSHYIHNLSSEMFSEFDKRYTHGRGFITKAINSCHTSSLATPEDKEQAQQMNQKDFLSLIVSILRSWNEPLYHLVTEVRGMQEAPEAILSKAVEIEEQTKRLLEGMELIVSQVHPETKENEIYPVWSGLPSLQMADEESRLSAYYNLLHCLRRDSHKIDNYLKLLKCRIIHNNNC

SEQ ID NO: 14 - protein, 240 aa, Artificial Sequence
  Synthesized
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIKGGGGGSGGGGSGGGGSDIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIK

SEQ ID NO: 15 - protein, 281 aa, Artificial Sequence
  Synthesized
MNVPTQLLGLLLLWLTGGKCDIQMTQSPASLSASLEEIVTITCKASQAIDAYLSWYQQKPGKSPQLLIYDATSLADGVPSRFSGSRSGTQYSLKISRPQVDDSGIYYCLQSYSTPFTFGSGTKLEIKGGGGGSGGGGSGGGGSMAVLVLLLCLLIFPSCVLSQVQLKESGPGLVQPSQTLSLTCTVSGSSLTSNSVSWIRQPPGKGLEWMGVIWSNGDADYNSAIKSRLSISRDTSKSQVFLKMNSLQTEDTAMYFCASPYYGYYFPFDYWGQGVMVTVSS

SEQ ID NO: 16 - protein, 285 aa, Artificial Sequence
  Synthesized
METDTLLLWVLLLWVPGSTGDIVLTQSPALAVSLGQRATISCRASQSVSISSHDLMQWYQQKPGQQPKLLIYDAFNLASGIPVRFSGSGSGTDFTLTIDPVQADDIATYYCQQSKDDPYTFGAGTKLELKGGGGGSGGGGSGGGGSMDIRLSLAFLVLFIKGVQCEVQLVESGGGLVQPGRSMKLSCAASGFTFSNYGMAWVRQAPTKGLEWVATISYDGSITYYRDSVKGRFTISRDHAKSTLYLQMNSLRSEDTATYYCTREEQYSSWYFDFWGPGIMVTVSS

SEQ ID NO: 17 - protein, 242 aa, Artificial Sequence
  Synthesized
NIVMTQSPSSLSASVGDRVTITCQASQDVGTAVAWYQQKPDQSPKLLIYWTSTRHTGVPDRFTGSGSGTDFTLTISSLQPEDIATYFCHQYNSYNTFGSGTKLEIKGGGGGSGGGGSGGGGSQVTLKESGPVLVKPTETLTLTCTFSGFSLSTSGMGVGWIRQAPGKGLEWVAHIWWDDDVYYNPSLKSRLTITKDASKDQVSLKLSSVTAADTAVYYCVRRRATGTGFDYWGQGTLVTVSS

SEQ ID NO: 18 - DNA, 339 bp, Homo sapiens
agagtgaagttcagcaggagcgcagacgcccccgcgtaccagcagggccagaaccagctctataacgagctcaatctaggacgaagagaggagtacgatgttttggacaagaggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 19 - DNA, 1689 bp, Artificial Sequence
  Synthesized
cggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggatccgccaccatggccttaccagtgaccgccttgctcctgccgctggccttgctgctccacgccgccaggccggacatccagctcacccagtccccgagctcgctgtccgcctccgtgggagatcgggtcaccatcacgtgccgcgccagccagtcgatttcctcctacctgaactggtaccaacagaagcccggaaaagccccgaagcttctcatctacgccgcctcgagcctgcagtcaggagtgccctcacggttctccggctccggttccggtactgatttcaccctgaccatttcctccctgcaaccggaggacttcgctacttactactgccagcagtcgtactccaccccctacactttcggacaaggcaccaaggtcgaaatcaagggtggcggtggctcgggcggtggtgggtcgggtggcggcggatctgaagtgcaattggtggaatcagggggaggacttgtgcagcctggaggatcgctgagactgtcatgtgccgtgtccggctttgccctgtccaaccacgggatgtcctgggtccgccgcgcgcctggaaagggcctcgaatgggtgtcgggtattgtgtacagcggtagcacctactatgccgcatccgtgaaggggagattcaccatcagccgggacaactccaggaacactctgtacctccaaatgaattcgctgaggccagaggacactgccatctactactgctccgcgcatggcggagagtccgacgtctggggacaggggaccaccgtgaccgtgtctagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagaggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 20 - DNA, 1446 bp, Artificial Sequence
  Synthesized
atggccttaccagtgaccgccttgctcctgccgctggccttgctgctccacgccgccaggccggacatccagctcacccagtccccgagctcgctgtccgcctccgtgggagatcgggtcaccatcacgtgccgcgccagccagtcgatttcctcctacctgaactggtaccaacagaagcccggaaaagccccgaagcttctcatctacgccgcctcgagcctgcagtcaggagtgccctcacggttctccggctccggttccggtactgatttcaccctgaccatttcctccctgcaaccggaggacttcgctacttactactgccagcagtcgtactccaccccctacactttcggacaaggcaccaaggtcgaaatcaagggtggcggtggctcgggcggtggtgggtcgggtggcggcggatctgaagtgcaattggtggaatcagggggaggacttgtgcagcctggaggatcgctgagactgtcatgtgccgtgtccggctttgccctgtccaaccacgggatgtcctgggtccgccgcgcgcctggaaagggcctcgaatgggtgtcgggtattgtgtacagcggtagcacctactatgccgcatccgtgaaggggagattcaccatcagccgggacaactccaggaacactctgtacctccaaatgaattcgctgaggccagaggacactgccatctactactgctccgcgcatggcggagagtccgacgtctggggacaggggaccaccgtgaccgtgtctagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagaggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 21 - DNA, 1467 bp, Artificial Sequence
  Synthesized
atggccttaccagtgaccgccttgctcctgccgctggccttgctgctccacgccgccaggccggacatcgtgatgacccagtccccctccagcctgacagtgacagccggcgagaaggtgacaatgatctgtaagtccagccagagcctgctgaacagcggcgaccagaagaactacctgacctggtaccagcagaagcctggccagccccccaagctgctgatcttctgggccagcacaagggagagcggcgtgcccgacagattcacaggcagcggcagcggcaccgacttcacactgaccatttcctccgtgcaggccgaggacctcgccgtgtactactgccagaacgactactcctaccccctgacattcggcgccggcaccaaactggagctgaagggtggcggtggctcgggcggtggtgggtcgggtggcggcggatctcaggtgcagctccagcagtccgatgccgagctggtgaagcccggaagcagcgtcaagatcagctgtaaggcctccggctacaccttcacagaccacgccatccactgggtgaagcagaagcccgagcagggcctggagtggatcggccactttagccccggaaacaccgacatcaagtacaacgacaagttcaagggcaaggccaccctgaccgtggacaggagcagcagcaccgcctacatgcagctgaacagcctgacaagcgaggacagcgccgtgtacttctgcaagacctccaccttcttcttcgactactggggccagggaaccaccctgacagtgtccagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagaggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 22 - DNA, 3533 bp, Artificial Sequence
  Synthesized
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagaggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaatctagaggcgcgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaacccatatgatggccttaccagtgaccgccttgctcctgccgctggccttgctgctccacgccgccaggccggacatcgtgatgacccagtccccctccagcctgacagtgacagccggcgagaaggtgacaatgatctgtaagtccagccagagcctgctgaacagcggcgaccagaagaactacctgacctggtaccagcagaagcctggccagccccccaagctgctgatcttctgggccagcacaagggagagcggcgtgcccgacagattcacaggcagcggcagcggcaccgacttcacactgaccatttcctccgtgcaggccgaggacctcgccgtgtactactgccagaacgactactcctaccccctgacattcggcgccggcaccaaactggagctgaagggtggcggtggctcgggcggtggtgggtcgggtggcggcggatctcaggtgcagctccagcagtccgatgccgagctggtgaagcccggaagcagcgtcaagatcagctgtaaggcctccggctacaccttcacagaccacgccatccactgggtgaagcagaagcccgagcagggcctggagtggatcggccactttagccccggaaacaccgacatcaagtacaacgacaagttcaagggcaaggccaccctgaccgtggacaggagcagcagcaccgcctacatgcagctgaacagcctgacaagcgaggacagcgccgtgtacttctgcaagacctccaccttcttcttcgactactggggccagggaaccaccctgacagtgtccagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagaggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 23 - DNA, 3533 bp, Artificial Sequence
  Synthesized
atggccttaccagtgaccgccttgctcctgccgctggccttgctgctccacgccgccaggccggatatccagatgacccagagcccgagcagcctgagcgcgagcgtgggtgatcgcgtgaccattacctgcagggcaagtcaggacattagtaaatatttaaattggtatcagcagaaaccgggtaaagcgccgaaactgttaatttatcatacatcaagattacactcaggcgtgccgtcgcgttttagcggctcgggttcgggcaccgattttaccctgaccatctcgagcttgcagccggaggacttcgccacctactattgccaacagggtaatacgcttccgtacacgttcggtcagggcaccaaagtggagatcaaaggtggcggtggctcgggcggtggtgggtcgggtggcggcggatctgaggtgcagctggtggagtctgggggaggcttggtacagcctggggggtccctgagactctcctgtgcagcctctggagtgtccctgcctgattatggcgtgtcctgggtccgccaggctccagggaaggggctggagtgggtttcagtgatctggggcagcgagacaacctactacaacagcgccctgaagtcccgattcaccatctccagagacaatgccaagaactcactgtatctgcaaatgaacagcctgagagccgaggacacggctgtgtattactgtgcgaagcactactactacggcggcagctacgctatggactactggggccaaggaaccctggtcaccgtgtcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagaggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaatctagaggcgcgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaacccatatgatggccttaccagtgaccgccttgctcctgccgctggccttgctgctccacgccgccaggccggacatcgtgatgacccagtccccctccagcctgacagtgacagccggcgagaaggtgacaatgatctgtaagtccagccagagcctgctgaacagcggcgaccagaagaactacctgacctggtaccagcagaagcctggccagccccccaagctgctgatcttctgggccagcacaagggagagcggcgtgcccgacagattcacaggcagcggcagcggcaccgacttcacactgaccatttcctccgtgcaggccgaggacctcgccgtgtactactgccagaacgactactcctaccccctgacattcggcgccggcaccaaactggagctgaagggtggcggtggctcgggcggtggtgggtcgggtggcggcggatctcaggtgcagctccagcagtccgatgccgagctggtgaagcccggaagcagcgtcaagatcagctgtaaggcctccggctacaccttcacagaccacgccatccactgggtgaagcagaagcccgagcagggcctggagtggatcggccactttagccccggaaacaccgacatcaagtacaacgacaagttcaagggcaaggccaccctgaccgtggacaggagcagcagcaccgcctacatgcagctgaacagcctgacaagcgaggacagcgccgtgtacttctgcaagacctccaccttcttcttcgactactggggccagggaaccaccctgacagtgtccagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagaggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 24 - DNA, 3545 bp, Artificial Sequence
  Synthesized
atggccttaccagtgaccgccttgctcctgccgctggccttgctgctccacgccgccaggccggatatccagatgacccagagcccaagctccctgtccgcctctgtgggcgacagggtgaccatcacatgccgcgccagccagacaatctggtcctacctgaactggtatcagcagagacccggcaaggcccctaatctgctgatctacgcagcatctagcctgcagtctggagtgccctcccggttctctggaagaggatccggaaccgacttcaccctgacaatctcctctctgcaggccgaggacttcgccacatactattgccagcagagctattccatccctcagacctttggccagggcacaaagctggagatcaagggcggcggcggctctggaggaggaggaagcggaggaggaggatcccaggtgcagctgcagcagagcggaccaggactggtgaagccctcccagaccctgtctctgacatgtgccatcagcggcgattccgtgagctccaacagcgccgcctggaattggatccggcagtctcccagcagaggactggagtggctgggaaggacctactatcgctccaagtggtacaacgattatgccgtgtctgtgaagagccggatcaccatcaaccctgacacatctaagaatcagttcagcctgcagctgaattccgtgaccccagaggacacagccgtgtactattgtgcaagggaggtgaccggcgacctggaggatgcctttgacatctggggccagggcaccatggtgacagtgtctagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagaggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaatctagaggcgcgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaacccatatgatggccttaccagtgaccgccttgctcctgccgctggccttgctgctccacgccgccaggccggacatcgtgatgacccagtccccctccagcctgacagtgacagccggcgagaaggtgacaatgatctgtaagtccagccagagcctgctgaacagcggcgaccagaagaactacctgacctggtaccagcagaagcctggccagccccccaagctgctgatcttctgggccagcacaagggagagcggcgtgcccgacagattcacaggcagcggcagcggcaccgacttcacactgaccatttcctccgtgcaggccgaggacctcgccgtgtactactgccagaacgactactcctaccccctgacattcggcgccggcaccaaactggagctgaagggtggcggtggctcgggcggtggtgggtcgggtggcggcggatctcaggtgcagctccagcagtccgatgccgagctggtgaagcccggaagcagcgtcaagatcagctgtaaggcctccggctacaccttcacagaccacgccatccactgggtgaagcagaagcccgagcagggcctggagtggatcggccactttagccccggaaacaccgacatcaagtacaacgacaagttcaagggcaaggccaccctgaccgtggacaggagcagcagcaccgcctacatgcagctgaacagcctgacaagcgaggacagcgccgtgtacttctgcaagacctccaccttcttcttcgactactggggccagggaaccaccctgacagtgtccagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagaggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 25 - DNA, 3518 bp, Artificial Sequence
  Synthesized
atggccttaccagtgaccgccttgctcctgccgctggccttgctgctccacgccgccaggccggacatccagctcacccagtccccgagctcgctgtccgcctccgtgggagatcgggtcaccatcacgtgccgcgccagccagtcgatttcctcctacctgaactggtaccaacagaagcccggaaaagccccgaagcttctcatctacgccgcctcgagcctgcagtcaggagtgccctcacggttctccggctccggttccggtactgatttcaccctgaccatttcctccctgcaaccggaggacttcgctacttactactgccagcagtcgtactccaccccctacactttcggacaaggcaccaaggtcgaaatcaagggtggcggtggctcgggcggtggtgggtcgggtggcggcggatctgaagtgcaattggtggaatcagggggaggacttgtgcagcctggaggatcgctgagactgtcatgtgccgtgtccggctttgccctgtccaaccacgggatgtcctgggtccgccgcgcgcctggaaagggcctcgaatgggtgtcgggtattgtgtacagcggtagcacctactatgccgcatccgtgaaggggagattcaccatcagccgggacaactccaggaacactctgtacctccaaatgaattcgctgaggccagaggacactgccatctactactgctccgcgcatggcggagagtccgacgtctggggacaggggaccaccgtgaccgtgtctagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagaggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaatctagaggcgcgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaacccatatgatggccttaccagtgaccgccttgctcctgccgctggccttgctgctccacgccgccaggccggacatcgtgatgacccagtccccctccagcctgacagtgacagccggcgagaaggtgacaatgatctgtaagtccagccagagcctgctgaacagcggcgaccagaagaactacctgacctggtaccagcagaagcctggccagccccccaagctgctgatcttctgggccagcacaagggagagcggcgtgcccgacagattcacaggcagcggcagcggcaccgacttcacactgaccatttcctccgtgcaggccgaggacctcgccgtgtactactgccagaacgactactcctaccccctgacattcggcgccggcaccaaactggagctgaagggtggcggtggctcgggcggtggtgggtcgggtggcggcggatctcaggtgcagctccagcagtccgatgccgagctggtgaagcccggaagcagcgtcaagatcagctgtaaggcctccggctacaccttcacagaccacgccatccactgggtgaagcagaagcccgagcagggcctggagtggatcggccactttagccccggaaacaccgacatcaagtacaacgacaagttcaagggcaaggccaccctgaccgtggacaggagcagcagcaccgcctacatgcagctgaacagcctgacaagcgaggacagcgccgtgtacttctgcaagacctccaccttcttcttcgactactggggccagggaaccaccctgacagtgtccagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagaggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 26 - DNA, 2994 bp, Artificial Sequence
  Synthesized
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagaggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctctagagccacgaacttctctctgttaaagcaagcaggagacgtggaagaaaaccccggtcctcatatgatggccttaccagtgaccgccttgctcctgccgctggccttgctgctccacgccgccaggccggacatcgtgatgacccagtccccctccagcctgacagtgacagccggcgagaaggtgacaatgatctgtaagtccagccagagcctgctgaacagcggcgaccagaagaactacctgacctggtaccagcagaagcctggccagccccccaagctgctgatcttctgggccagcacaagggagagcggcgtgcccgacagattcacaggcagcggcagcggcaccgacttcacactgaccatttcctccgtgcaggccgaggacctcgccgtgtactactgccagaacgactactcctaccccctgacattcggcgccggcaccaaactggagctgaagggtggcggtggctcgggcggtggtgggtcgggtggcggcggatctcaggtgcagctccagcagtccgatgccgagctggtgaagcccggaagcagcgtcaagatcagctgtaaggcctccggctacaccttcacagaccacgccatccactgggtgaagcagaagcccgagcagggcctggagtggatcggccactttagccccggaaacaccgacatcaagtacaacgacaagttcaagggcaaggccaccctgaccgtggacaggagcagcagcaccgcctacatgcagctgaacagcctgacaagcgaggacagcgccgtgtacttctgcaagacctccaccttcttcttcgactactggggccagggaaccaccctgacagtgtccagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagaggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 27 - DNA, 2994 bp, Artificial Sequence
  Synthesized
atggccttaccagtgaccgccttgctcctgccgctggccttgctgctccacgccgccaggccggatatccagatgacccagagcccgagcagcctgagcgcgagcgtgggtgatcgcgtgaccattacctgcagggcaagtcaggacattagtaaatatttaaattggtatcagcagaaaccgggtaaagcgccgaaactgttaatttatcatacatcaagattacactcaggcgtgccgtcgcgttttagcggctcgggttcgggcaccgattttaccctgaccatctcgagcttgcagccggaggacttcgccacctactattgccaacagggtaatacgcttccgtacacgttcggtcagggcaccaaagtggagatcaaaggtggcggtggctcgggcggtggtgggtcgggtggcggcggatctgaggtgcagctggtggagtctgggggaggcttggtacagcctggggggtccctgagactctcctgtgcagcctctggagtgtccctgcctgattatggcgtgtcctgggtccgccaggctccagggaaggggctggagtgggtttcagtgatctggggcagcgagacaacctactacaacagcgccctgaagtcccgattcaccatctccagagacaatgccaagaactcactgtatctgcaaatgaacagcctgagagccgaggacacggctgtgtattactgtgcgaagcactactactacggcggcagctacgctatggactactggggccaaggaaccctggtcaccgtgtcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagaggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctctagagccacgaacttctctctgttaaagcaagcaggagacgtggaagaaaaccccggtcctcatatgatggccttaccagtgaccgccttgctcctgccgctggccttgctgctccacgccgccaggccggacatcgtgatgacccagtccccctccagcctgacagtgacagccggcgagaaggtgacaatgatctgtaagtccagccagagcctgctgaacagcggcgaccagaagaactacctgacctggtaccagcagaagcctggccagccccccaagctgctgatcttctgggccagcacaagggagagcggcgtgcccgacagattcacaggcagcggcagcggcaccgacttcacactgaccatttcctccgtgcaggccgaggacctcgccgtgtactactgccagaacgactactcctaccccctgacattcggcgccggcaccaaactggagctgaagggtggcggtggctcgggcggtggtgggtcgggtggcggcggatctcaggtgcagctccagcagtccgatgccgagctggtgaagcccggaagcagcgtcaagatcagctgtaaggcctccggctacaccttcacagaccacgccatccactgggtgaagcagaagcccgagcagggcctggagtggatcggccactttagccccggaaacaccgacatcaagtacaacgacaagttcaagggcaaggccaccctgaccgtggacaggagcagcagcaccgcctacatgcagctgaacagcctgacaagcgaggacagcgccgtgtacttctgcaagacctccaccttcttcttcgactactggggccagggaaccaccctgacagtgtccagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagaggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 28 - DNA, 3009 bp, Artificial Sequence
  Synthesized
atggccttaccagtgaccgccttgctcctgccgctggccttgctgctccacgccgccaggccggatatccagatgacccagagcccaagctccctgtccgcctctgtgggcgacagggtgaccatcacatgccgcgccagccagacaatctggtcctacctgaactggtatcagcagagacccggcaaggcccctaatctgctgatctacgcagcatctagcctgcagtctggagtgccctcccggttctctggaagaggatccggaaccgacttcaccctgacaatctcctctctgcaggccgaggacttcgccacatactattgccagcagagctattccatccctcagacctttggccagggcacaaagctggagatcaagggcggcggcggctctggaggaggaggaagcggaggaggaggatcccaggtgcagctgcagcagagcggaccaggactggtgaagccctcccagaccctgtctctgacatgtgccatcagcggcgattccgtgagctccaacagcgccgcctggaattggatccggcagtctcccagcagaggactggagtggctgggaaggacctactatcgctccaagtggtacaacgattatgccgtgtctgtgaagagccggatcaccatcaaccctgacacatctaagaatcagttcagcctgcagctgaattccgtgaccccagaggacacagccgtgtactattgtgcaagggaggtgaccggcgacctggaggatgcctttgacatctggggccagggcaccatggtgacagtgtctagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagaggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaatctagagccacgaacttctctctgttaaagcaagcaggagacgtggaagaaaaccccggtcctcatatgatggccttaccagtgaccgccttgctcctgccgctggccttgctgctccacgccgccaggccggacatcgtgatgacccagtccccctccagcctgacagtgacagccggcgagaaggtgacaatgatctgtaagtccagccagagcctgctgaacagcggcgaccagaagaactacctgacctggtaccagcagaagcctggccagccccccaagctgctgatcttctgggccagcacaagggagagcggcgtgcccgacagattcacaggcagcggcagcggcaccgacttcacactgaccatttcctccgtgcaggccgaggacctcgccgtgtactactgccagaacgactactcctaccccctgacattcggcgccggcaccaaactggagctgaagggtggcggtggctcgggcggtggtgggtcgggtggcggcggatctcaggtgcagctccagcagtccgatgccgagctggtgaagcccggaagcagcgtcaagatcagctgtaaggcctccggctacaccttcacagaccacgccatccactgggtgaagcagaagcccgagcagggcctggagtggatcggccactttagccccggaaacaccgacatcaagtacaacgacaagttcaagggcaaggccaccctgaccgtggacaggagcagcagcaccgcctacatgcagctgaacagcctgacaagcgaggacagcgccgtgtacttctgcaagacctccaccttcttcttcgactactggggccagggaaccaccctgacagtgtccagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagaggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 29 - DNA, 2982 bp, Artificial Sequence
  Synthesized
atggccttaccagtgaccgccttgctcctgccgctggccttgctgctccacgccgccaggccggacatccagctcacccagtccccgagctcgctgtccgcctccgtgggagatcgggtcaccatcacgtgccgcgccagccagtcgatttcctcctacctgaactggtaccaacagaagcccggaaaagccccgaagcttctcatctacgccgcctcgagcctgcagtcaggagtgccctcacggttctccggctccggttccggtactgatttcaccctgaccatttcctccctgcaaccggaggacttcgctacttactactgccagcagtcgtactccaccccctacactttcggacaaggcaccaaggtcgaaatcaagggtggcggtggctcgggcggtggtgggtcgggtggcggcggatctgaagtgcaattggtggaatcagggggaggacttgtgcagcctggaggatcgctgagactgtcatgtgccgtgtccggctttgccctgtccaaccacgggatgtcctgggtccgccgcgcgcctggaaagggcctcgaatgggtgtcgggtattgtgtacagcggtagcacctactatgccgcatccgtgaaggggagattcaccatcagccgggacaactccaggaacactctgtacctccaaatgaattcgctgaggccagaggacactgccatctactactgctccgcgcatggcggagagtccgacgtctggggacaggggaccaccgtgaccgtgtctagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagaggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaatctagagccacgaacttctctctgttaaagcaagcaggagacgtggaagaaaaccccggtcctcatatgatggccttaccagtgaccgccttgctcctgccgctggccttgctgctccacgccgccaggccggacatcgtgatgacccagtccccctccagcctgacagtgacagccggcgagaaggtgacaatgatctgtaagtccagccagagcctgctgaacagcggcgaccagaagaactacctgacctggtaccagcagaagcctggccagccccccaagctgctgatcttctgggccagcacaagggagagcggcgtgcccgacagattcacaggcagcggcagcggcaccgacttcacactgaccatttcctccgtgcaggccgaggacctcgccgtgtactactgccagaacgactactcctaccccctgacattcggcgccggcaccaaactggagctgaagggtggcggtggctcgggcggtggtgggtcgggtggcggcggatctcaggtgcagctccagcagtccgatgccgagctggtgaagcccggaagcagcgtcaagatcagctgtaaggcctccggctacaccttcacagaccacgccatccactgggtgaagcagaagcccgagcagggcctggagtggatcggccactttagccccggaaacaccgacatcaagtacaacgacaagttcaagggcaaggccaccctgaccgtggacaggagcagcagcaccgcctacatgcagctgaacagcctgacaagcgaggacagcgccgtgtacttctgcaagacctccaccttcttcttcgactactggggccagggaaccaccctgacagtgtccagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagaggcgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 30 - protein, 244 aa, Artificial Sequence
  Synthesized
DIVMTQSPSSLTVTAGEKVTMICKSSQSLLNSGDQKNYLTWYQQKPGQPPKLLIFWASTRESGVPDRFTGSGSGTDFTLTISSVQAEDLAVYYCQNDYSYPLTFGAGTKLELKGGGGSGGGGSGGGGSQVQLQQSDAELVKPGSSVKISCKASGYTFTDHAIHWVKQKPEQGLEWIGHFSPGNTDIKYNDKFKGKATLTVDRSSSTAYMQLNSLTSEDSAVYFCKTSTFFFDYWGQGTTLTVSS

SEQ ID NO: 31 - protein, 116 aa, Artificial Sequence
  Synthesized
QVQLQQSDAELVKPGSSVKISCKASGYTFTDHAIHWVKQKPEQGLEWIGHFSPGNTDIKYNDKFKGKATLTVDRSSSTAYMQLNSLTSEDSAVYFCKTSTFFFDYWGQGTTLTVSS

SEQ ID NO: 32 - protein, 113 aa, Artificial Sequence
  Synthesized
DIVMTQSPSSLTVTAGEKVTMICKSSQSLLNSGDQKNYLTWYQQKPGQPPKLLIFWASTRESGVPDRFTGSGSGTDFTLTISSVQAEDLAVYYCQNDYSYPLTFGAGTKLELK

SEQ ID NO: 33 - protein, 246 aa, Artificial Sequence
  Synthesized
DIVMTQAAFSNPVTLGTSASISCRSSKSLLHSNGITYFFWYLQKPGLSPQLLIYQMSNLASGVPDRFSSSGSGTDFTLRISRVEAEDVGVYYCAQNLELPPTFGGGTKLEIKRAGGGGSGGGGSGGGGSEVKLVESGGGLVQPGGSMKLSCVASGFTFSNYWMNWVRQSPEKGLEWVAEIRLKSNNYTTHYAESVKGRFTISRDDSKSSVSLQMNNLRVEDTGIYYCTRHYYFDYWGQGTTLTVSS

SEQ ID NO: 34 - protein, 117 aa, Artificial Sequence
  Synthesized
EVKLVESGGGLVQPGGSMKLSCVASGFTFSNYWMNWVRQSPEKGLEWVAEIRLKSNNYTTHYAESVKGRFTISRDDSKSSVSLQMNNLRVEDTGIYYCTRHYYFDYWGQGTTLTVSS

SEQ ID NO: 35 - protein, 114 aa, Artificial Sequence
  Synthesized
DIVMTQAAFSNPVTLGTSASISCRSSKSLLHSNGITYFFWYLQKPGLSPQLLIYQMSNLASGVPDRFSSSGSGTDFTLRISRVEAEDVGVYYCAQNLELPPTFGGGTKLEIKRA

SEQ ID NO: 36 - protein, 97 aa, Artificial Sequence
  Synthesized
CSRAARGTIGARRTGQPLKEDPSAVPVFSVDAGELDFQWREKTPEPPVPCVPEQTEAATIVFPSGMGTSSPARRGSADGPRSAQPLRPEDGHCSWPL

SEQ ID NO: 37 - protein, 288 aa, Artificial Sequence
  Synthesized
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDAGELDFQWREKTPEPPVPCVPEQTEAATIVFPSGMGTSSPARRGSADGPRSAQPLRPEDGHCSWPL

SEQ ID NO: 38 - protein, 17 aa, Artificial Sequence
  Synthesized
CSRAARGTIDGHCSWPL

SEQ ID NO: 39 - protein, 21 aa, Artificial Sequence
  Synthesized
VGVVGGLLGSLVLLVWVLAVI

SEQ ID NO: 40 - protein, 18 aa, Artificial Sequence
  Synthesized
GNILNVSIKICLTLSPST

SEQ ID NO: 41 - protein, 9 aa, Artificial Sequence
  Synthesized
CSRAARGTI

SEQ ID NO: 42 - protein, 80 aa, Artificial Sequence
  Synthesized
GARRTGQPLKEDPSAVPVFSVDYGELDFQWREKTPEPPVPCVPEQTEYATIVFPSGMGTSSPARRGSADGPRSAQPLRPE

SEQ ID NO: 43 - protein, 69 aa, Artificial Sequence
  Synthesized
TTTPAPRPPTPAPTIASQPLSLRPEACRPAAGGAVHTRGLDFACDIYIWAPLAGTCGVLLLSLVITLYC

SEQ ID NO: 44 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 45 - protein, 223 aa, Artificial Sequence
  Synthesized
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVFVKMPPTEPECEKQFQPYFIPIN

SEQ ID NO: 46 - protein, 223 aa, Artificial Sequence
  Synthesized
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPFFIPIN

SEQ ID NO: 47 - protein, 223 aa, Artificial Sequence
  Synthesized
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVFVKMPPTEPECEKQFQPFFIPIN

SEQ ID NO: 48 - protein, 189 aa, Artificial Sequence
  Synthesized
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKM

SEQ ID NO: 49 - protein, 240 aa, Artificial Sequence
  Synthesized
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDAAAPTTTPAPRPPTPAPTIASQPLSLRPEACRPAAGGAVHTRGLDFACDIYIWAPLAGTCGVLLLSLVITLYCNHRRIQ

SEQ ID NO: 50 - protein, 223 aa, Artificial Sequence
  Synthesized
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYAGIGNGTQIYVIDPEPCPDSDFLLWILAAVSSGLFFYSFLLTAVSLSKMLKKRSPLTTGVFVKMPPTEPECEKQFQPFFIPIN

SEQ ID NO: 51 - protein, 189 aa, Artificial Sequence
  Synthesized
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYAGIGNGTQIYVIDPEPCPDSDFLLWILAAVSSGLFFYSFLLTAVSLSKM

SEQ ID NO: 52 - protein, 240 aa, Artificial Sequence
  Synthesized
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYAGIGNGTQIYVIDPEPCPDSDAAAPTTTPAPRPPTPAPTIASQPLSLRPEACRPAAGGAVHTRGLDFACDIYIWAPLAGTCGVLLLSLVITLYCNHRRIQ

SEQ ID NO: 53 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 54 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 55 - protein, 288 aa, Artificial Sequence
  Synthesized
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCSAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 56 - protein, 288 aa, Artificial Sequence
  Synthesized
MQIPQAPWPVVWAVLQLGWRPGWFLDSPDRPWNPPTFSPALLVVTEGDNATFTCSFSNTSESFVLNWYRISPSNQTDKLAAFPEDRSQPGQDCRFRVTQLPNGRDFHMSVVRARRNDSGTYLCSAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 57 - protein, 288 aa, Artificial Sequence
  Synthesized
MQIPQAPWPVVWAVLQLGWRPGWFLDSPDRPWNPPTFSPALLVVTEGDNATFTCSFSNTSESFVLNWYRMSPSNQTVKLAAFPEDRSQPGQDCRFRVTQLPNGRDFHMSVVRARRNDSGTYLCSAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 58 - protein, 288 aa, Artificial Sequence; identical to SEQ ID NO: 37
  synthesized

SEQ ID NO: 59 - protein, 200 aa, Artificial Sequence
  synthesized
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTI

SEQ ID NO: 60 - protein, 15 aa, Artificial Sequence
  Synthesized
GGGGSGGGGSGGGGS